(19) United States
(12) Patent Application Publication
(10) Pub. No.: US 2011/0118269 A1
(43) Pub. Date: May 19, 2011

(54) FUSED PYRAZINE COMPOUNDS AS THEIR SALTS, USEFUL FOR THE TREATMENT OF DEGENERATIVE AND INFLAMMATORY DISEASES

(76) Inventors: Piet Tom Bert Paul WIGERINCK, Mechelen (BE); Martin James Inglis ANDREWS, Mechelen (BE); Marc Maurice Germain De WEER, Mechelen (BE); Nicholas Luc SABOURAULT, Romainville (FR); Stefan Christian KLUGE, Basel (CH)

(21) Appl. No.: 12/940,710

(22) Filed: Nov. 5, 2010

Related U.S. Application Data
(60) Provisional application No. 61/258,258, filed on Nov. 5, 2009.

(51) Int. Cl. A61K 31/4985; C07D 487/08; A61P 29/00; A61P 9/10; A61P 17/06; A61P 37/06; A61P 19/02

(52) U.S. Cl. 514/249; 544/350

(57) ABSTRACT

Novel salts of a [1.2.4]triazolo[1,5-a]pyrazine compound according to Formula I:

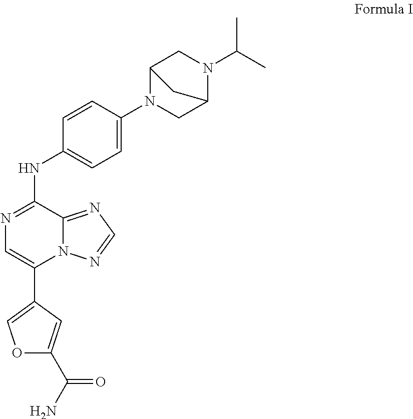

The salts may be prepared as pharmaceutical compositions, and may be used for the prevention and treatment of a variety of conditions in mammals including humans, including by way of non-limiting example, inflammation, and others.

### RELATED APPLICATIONS

The present application claims the benefit under 35 U.S.C. §119 of U.S. Provisional Application No. 61/258,258, filed Nov. 5, 2009, the contents of which is hereby incorporated herein by reference in its entirety.

### FIELD OF THE INVENTION

The present invention relates to a class of fused pyrazine compounds as their salt forms capable of binding to the active site of a serine/threonine kinase, the expression of which is involved in the pathway resulting in the degradation of extra-cellular matrix (ECM), joint degeneration and diseases involving such degradation and/or inflammation.

Diseases involving the degradation of extra-cellular matrix include, but are not limited to, psoriatic arthritis, juvenile arthritis, early arthritis, reactive arthritis, osteoarthritis, rheumatoid arthritis, ankylosing spondylitis, osteoporosis, musculo skeletal diseases like tendonitis and periodontal disease, cancer metastasis, airway diseases (COPD, asthma), renal and liver fibrosis, cardio-vascular diseases like atherosclerosis and heart failure, and neurological diseases like neuroinflammation and multiple sclerosis. Diseases involving primarily joint degeneration include, but are not limited to, psoriatic arthritis, juvenile arthritis, early arthritis, reactive arthritis, rheumatoid arthritis, osteoarthritis, and ankylosing spondylitis.

Rheumatoid arthritis (RA) is a chronic joint degenerative disease, characterized by inflammation and destruction of the joint structures. When the disease is unchecked, it leads to substantial disability and pain due to loss of joint functionality and even premature death. The aim of an RA therapy, therefore, is not to slow down the disease but to attain remission in order to stop the joint destruction. Besides the severity of the disease outcome, the high prevalence of RA (˜0.8% of adults are affected worldwide) means a high socio-economic impact. (For reviews on RA, we refer to Smolen and Steiner (2003); Lee and Weinblatt (2001); Choy and Panayi (2001); O'Dell (2004) and Firestein (2003)).

Although it is widely accepted that RA is an auto-immune disease, there is no consensus concerning the precise mechanisms driving the ‘initiation stage’ of the disease. What is known is that the initial trigger(s) does mediate, in a predisposed host, a cascade of events that leads to the activation of various cell types (B-cells, T-cells, macrophages, fibroblasts, endothelial cells, dendritic cells and others). Concomitantly, an increased production of various cytokines is observed in the joints and tissues surrounding the joint (e.g. TNF-α, IL-6, IL-1, IL-15, IL-18 and others). When the disease progresses, the cellular activation and cytokine production cascade becomes self-perpetuating. At this early stage, the destruction of joint structures is already very clear. Thirty percent of the patients have radiographic evidence of bony erosions at the time of diagnosis and this proportion increases to 60 percent after two years.

Histological analysis of the joints of RA patients clearly evidences the mechanisms involved in the RA-associated degradative processes. This analysis shows that the main effector responsible for RA-associated joint degradation is the pannus, where the synovial fibroblast, by producing diverse proteolytic enzymes, is the prime driver of cartilage and bone erosion. A joint classically contains two adjacent bones that articulate on a cartilage layer and are surrounded by the synovial membrane and joint capsule. In the advanced RA patient, the synovium of the joint increases in size to form the pannus, due to the proliferation of the synovial fibroblasts and the infiltration of mononuclear cells such as T-cells, B-cells, monocytes, macrophages and neutrophils. The pannus mediates the degradation of the adjacent cartilage, leading to the narrowing of the joint space, and has the potential to invade adjacent bone and cartilage. As bone and cartilage tissues are composed mainly of collagen type I or II, respectively, the pannus destructive and invasive properties are mediated by the secretion of collagenolytic proteases, principally the matrix metallo proteinases (MMPs). The erosion of the bone under and adjacent to the cartilage is also part of the RA process, and results principally from the presence of osteoclasts at the interface of bone and pannus. Osteoclasts are multinucleated cells that, upon adhesion to the bone tissue, form a closed compartment, within which the osteoclasts secrete proteases (Cathepsin K, MMP9) that degrade the bone tissue. The osteoclast population in the joint is abnormally increased by osteoblast formation from precursor cells induced by the secretion of the receptor activator of NFκB ligand (RANKL) by activated SFs and T-cells.

Various collagen types have a key role in defining the stability of the extracellular matrix (ECM). Collagen type I and collagen type II, for example, are the main components of bone and cartilage, respectively. Collagen proteins typically organise into multimeric structures referred to as collagen fibrils. Native collagen fibrils are very resistant to proteolytic cleavage. Only a few types of ECM-degrading proteins have been reported to have the capacity to degrade native collagen: MMPs and Cathepsins. Among the Cathepsins, cathepsin K, which is active mainly in osteoclasts, is the best characterised. Among the MMPs, MMP1, MMP2, MMP8 MMP13 and MMP14 are known to have collagenolytic properties. The correlation between an increased expression of MMP1 by synovial fibroblasts (SFs) and the progression of the arthritic disease is well-established and is predictive for joint erosive processes (Cunnane et al., 2001). In the context of RA, therefore, MMP1 represents a highly relevant collagen degrading protein. In vitro, the treatment of cultured SFs with cytokines relevant in the RA pathology (e.g. TNF-α and IL1β) will increase the expression of MMP1 by these cells (Andreakos et al., 2003). Monitoring the levels of MMP1 expressed by SFs therefore is a relevant readout in the field of RA as it is indicative for the activation of SFs towards an erosive phenotype that, in vivo, is responsible for cartilage degradation. Inhibition of the MMP1 expression by SFs represents a valuable therapeutic approach towards the treatment of RA.

The activity of the ECM-degrading proteins can also be causative or correlate with the progression of various diseases different from RA, as e.g. other diseases that involve the degradation of the joints. These diseases include, but are not limited to, psoriatic arthritis, juvenile arthritis, early arthritis, reactive arthritis, osteoarthritis, and ankylosing spondylitis. Other diseases that may be treatable with compounds identified according to the present invention and using the targets involved in the expression of MMPs as described herein are osteoporosis, muscular skeletal diseases like tendonitis and periodontal disease (Gapski et al., 2004), cancer metastasis (Coussens et al., 2002), airway diseases (COPD, asthma) (Suzuki et al., 2004), lung, renal fibrosis (Schanstra et al., 2002), liver fibrosis associated with chronic hepatitis C (Reiff et al., 2005), cardio-vascular diseases like atherosclerosis and heart failure (Creemers et al., 2001), and neurological diseases like neuroinflammation and multiple sclerosis (Rosenberg, 2002). Patients suffering from such diseases may benefit from stabilizing the ECM (by protecting it from degradation).

The 471-amino acid serine/threonine kinase identified as Mitogen-Activated Protein Kinase-Activated Protein Kinase 5 (MAPKAPK5 or PRAK) is expressed in a wide panel of tissues. The protein contains its catalytic domain at the N-terminal end and both a nuclear localization signal (NLS) and nuclear export signal (NES) at its C-terminal end. Endogenous MAPKAPK5 is predominantly present in the cytoplasm, but stress or cytokine activation of the cells mediates its translocation into the nucleus (New et al., 2003). This event is dependent on phosphorylation of MAPKAPK5. Thr182 is the regulatory phosphorylation site of MAPKAPK5. Although the p38α kinase is able to phosphorylate MAPKAPK5 in an overexpression setting, experiments with endogenous MAPKAPK5 do not support this hypothesis (Shi et al., 2003). MAPKAPK5 knock-out mice have been generated that are viable and fertile. The phenotype of these mice is quite different from that of mice deficient for MAPKAPK2, a MAPKAPK5 related kinase that is regulated by p38α (Shi et al., 2003). This indicates that the function of each protein is distinct and that neither kinase can compensate for the other's activity. Taken together, MAPKAPK5 and MAPKAPK2 represent distinct targets with a non-redundant role. MAPK6 (also referred to as ERK3) has recently been identified as a physiologically relevant substrate for MAPKAPK5, defining a novel signal transduction pathway (Seternes et al., 2004).

### BACKGROUND OF THE INVENTION

NSAIDS (Non-steroidal anti-inflammatory drugs) are used to reduce the pain associated with RA and improve life quality of the patients. These drugs will not, however, put a brake on the RA-associated joint destruction.

Corticosteroids were found to decrease the progression of RA as detected radiographically and are used at low doses to treat part of the RA patients (30 to 60%). Serious side effects, however, are associated with long corticosteroid use (skin thinning, osteoporosis, cataracts, hypertension, and hyperlipidemia).

Synthetic DMARDs (Disease-Modifying Anti-Rheumatic Drugs) (e.g. methotrexate, leflunomide, sulfasalazine, auranofin, sodium aurothiomalate, penicillamine, chloroquine, hydroxychloroquine, azathioprine, and ciclosporin) mainly tackle the immuno-inflammatory component of RA. As a main disadvantage, these drugs only have a limited efficacy (joint destruction is only slowed down but not blocked by DMARDs such that disease progression in the long term continues). The lack of efficacy is indicated by the fact that, on average, only 30% of the patients achieve an ACR50 score after 24 months treatment with methotrexate. This means that, according to the American College of Rheumatology, only 30% of the patients do achieve a 50% improvement of their symptoms (O'Dell et al., 1996). In addition, the precise mechanism of action of DMARDs is often unclear.

Biological DMARDs (Infliximab, Etanercept, Adalimumab, Rituximab, Abatacept) are therapeutic proteins that do inactivate cytokines (e.g. TNF-α) or cells (e.g. B-cells or T-cells) that have an important role in the RA pathophysiology (Kremer et al., 2003; Edwards et al., 2004). Although the TNF-α-blockers (Infliximab, Etanercept, Adalimumab) and methotrexate combination therapy is the most effective RA treatment currently available, it is striking that even this therapy only achieves a 50% improvement (ACR50) in disease symptoms in 50-60% of patients after 12 months therapy (St Clair et al., 2004). Some adverse events warnings for anti-TNF-α drugs exist, shedding a light on the side effects associated to this type of drugs. Increased risk for infections (tuberculosis), hematologic events and demyelinating disorders have been described for the TNF-α blockers (see also Gomez-Reino et al., 2003). Besides the serious side effects, the TNF-α blockers do also share the general disadvantages of the biological class of therapeutics, which are the unpleasant way of administration (frequent injections accompanied by infusion site reactions) and the high production cost. Newer agents in late development phase target cytokines such as IL-6, T-cell co-stimulatory molecules and B-cells. The efficacy of these agents is expected to be similar to that of the TNF-α blockers. The fact that a variety of targeted therapies have similar but limited efficacies, suggests that there is a multiplicity of pathogenic factors for RA. This is also indicative for the deficiencies in our understanding of pathogenic events relevant to RA.

The current therapies for RA are not satisfactory due to a limited efficacy (no adequate therapy exists for 30% of the patients). This calls for additional strategies to achieve remission. Remission is required since residual disease bears the risk of progressive joint damage and thus progressive disability. Inhibiting the immuno-inflammatory component of the RA disease, which represents the main target of drugs currently used for RA treatment, does not result in a blockade of joint degradation, the major hallmark of the disease.

US 2005/0009832 describes substituted imidazo[1,2-a]pyrazine-8-yl-amines as modulators of protein kinases, including MAPKAPK5. WO02/056888 describes inhibitors of MAPKAPK5 as TNF modulators able to regulate the expression of certain cytokines. Neither of these prior art references discloses any compound within the scope of the class of compounds described herein below.

Furthermore, an important characteristic of various bioactive substances (for example but without limitation pharmaceuticals, medicines and biocides, usually referred to as drugs) is their “bio-availability” or active concentration in a form which can be absorbed and utilized by a target organ or organism. In many cases, the bioavailability is related to the drug solubility in water.

To be of useful as a therapeutic agent, the drug should be available in the soluble form in a proper concentration range for a required period of time. Various options are available to achieve these properties, including formulating the drug as a pill, capsules, solutions, ointments, or other similar formulations. Of particular interest are “zero-order release” drugs, in which the rate of drug release is constant. However, developing these systems is often complicated and expensive.

Often, drugs in their free base form are poorly soluble in water, but the presence of acidic sites (for example carboxylic acids, phenols, sulfonic acids) or basic sites (for example amino groups, basic nitrogen centres) can be used advantageously to produce salts of the drug. The resulting ionic compounds become much more soluble in water by virtue of their ionic character and lower dissolution energy, and thus improve bioavailability. A guideline of 50 μg/mL for aqueous solubility was provided by Lipinsky et al. (Lipinsky et al. Adv. Drug Del. Rev., 1997, 23, 3-25).

Selecting the best salt form for development of a drug candidate is critical in obtaining a robust product, and requires experimental investigations in order to make the best choice.

Moreover, selecting an appropriate salt form for a drug candidate provides a way to modulate the characteristics of the drug, and also to improve the bioavailability, stability, manufacturability and patient compliance. Salt forming agents are available in large number, and salt selection must be carefully designed. The aim of the salt selection is to identify the best salt form suitable for development, and is based primarily on four main criteria: aqueous solubility at various pH, high degree of crystallinity, low hygroscopy, and optimal chemical stability. Additional criteria also include the limited formation of polymorphs, and easy synthesis (Handbook of Pharmaceutical Salts: Properties, Selection and Use, Stahl, P. H. and Wermuth, C. G. Eds. Wiley-VCH, Weinheim, Germany, 2002).

Thus the object of this invention is to disclose salt forms of the compounds of the invention, which have desirable pharmacological properties, which salt forms in at least some embodiments are expected to exhibit improvements in their pharmaceutical profile compared to the free base form of the compound.

### SUMMARY OF THE INVENTION

The present invention is based on the discovery that MAPKAPK5 functions in the pathway that results in the expression of MMP1, and that inhibitors of MAPKAPK5 activity, such as the compounds of the present invention, are useful for the treatment of diseases involving the abnormally high expression of MMP activity.

The salts of the invention may be described generally as salts of a [1.2.4]triazolo[1,5-a]pyrazine, substituted in the 5-position by a 3-amido-furan-4-yl, and an in the 8-position by a substituted 4-(diazabicyclo[2.2.1]heptan-2-yl)-4-aniline group.

The salts of the invention may show one or more of the following advantageous properties: high crystallinity, improved processability, improved chemical stability, low hygroscopy, lower dissolution energy, better absorption, less toxicity, good absorption, good half-life, good solubility, low protein binding affinity, less drug-drug interaction, and good metabolic stability. In a particular aspect, the compounds of the present invention exhibit unexpected significant improvements in pharmacological properties over similar compounds. In particular they may exhibit improved efficacy, improved stability, improved solubility, improved processability and improved tolerability, which improvements are also reflected in their salt forms. In at least some embodiments, the salts of the present invention are expected to exhibit unexpected significant improvements in chemical stability and/or solubility over the corresponding free base.

More particularly, the present invention relates to salts of the compound according to Formula I:

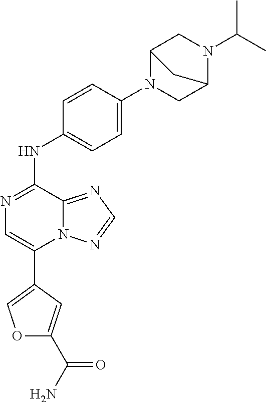

wherein said salt is a salt formed with adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, or toluenesulfonic acid. In a preferred aspect of the invention the salt of the invention is the salt formed with benzoic acid, fumaric acid, methanesulfonic acid, or citric acid. In an alternative embodiment, the salt of the invention is the salt formed with benzenesulfonic acid, naphthalene-1,5-disulfonic acid or toluene sulfonic acid.

In a further aspect, the present invention provides pharmaceutical compositions comprising a salt of the invention, and a pharmaceutical carrier, excipient or diluent. In this aspect of the invention, the pharmaceutical composition can comprise one or more of the salts of the invention described herein. Moreover, the salts of the invention useful in the pharmaceutical compositions and treatment methods disclosed herein, are all pharmaceutically acceptable as prepared and used.

Another aspect of this invention relates to the use of a salt of the invention in a therapeutic method, a pharmaceutical composition, and the manufacture of such composition, useful for the treatment of diseases involving inflammation, collagen degradation, and in particular, diseases characteristic of abnormal matrix metallo protease (MMP1) and/or Mitogen-Activated Protein-Kinase Activated Protein Kinase 5 (MAPKAPK5) activity, of which rheumatoid arthritis (RA) is a particular such disease. This invention also relates to processes for the preparation of the salts of the invention.

Other objects and advantages will become apparent to those skilled in the art from a consideration of the ensuing detailed description, considered in conjunction with the following illustrative drawings.

### BRIEF DESCRIPTION OF THE DRAWINGS

FIG. 1. This diagram shows the striking histological differences between a healthy joint and that of a RA patient.

FIG. 2. This chart shows the increased expression of MMP1 in synovial fibroblasts triggered with cytokines involved in rheumatoid arthritis pathology.

FIG. 3. This graph shows the dose-dependent inhibition of the “TNF-α-based trigger”-induced expression of MMP1 by SFs by a known anti-inflammatory compound.

FIG. 4. This gel shows the reduction, at the protein level, of the expression of MAPKAPK5 in SFs by infection of the cells with Ad-siRNA virus targeting MAPKAPK5.

FIG. 5. This chart shows the reduction of ‘complex trigger’ induced levels of MMP1 expression by SFs by an Ad-siRNA virus targeting MAPKAPK5.

FIG. 6A This graph shows the results of tolerability study conducted with Compound 1, where the measured effect was against total body weight.

FIG. 6B This graph shows the results of tolerability study against a comparator compound.

FIG. 7 This graph shows the percentage inhibition of TNF alpha release obtained with Compound 1, after injection of LPS (bacterial lipopolysaccharides).

FIG. 8A This graph shows the results of DVS study for mesylate salt of Compound 1.

FIG. 8B This graph shows the results of DVS study for fumarate salt of Compound 1.

FIG. 8C This graph shows the results of GVS study for besylate salt of Compound 1.

FIG. 9A This graph represents the Raman spectrum of Compound 1.

FIG. 9B This graph represents the PXRD spectrum of Compound 1.

FIG. 9C This graph represents the Raman spectrum of Compound 1 as a fumarate salt.

FIG. 9D This graph represents the PXRD spectrum of Compound 1 as a fumarate salt.

FIG. 9E This graph represents the Raman spectrum of Compound 1 as a mesylate salt.

FIG. 9F This graph represents the PXRD spectrum of Compound 1 as a mesylate salt.

FIG. 9G This graph represents the PXRD spectrum of Compound 1 as a besylate salt obtained for salt screening.

FIG. 10A This graph represents the PXRD spectrum of Compound 1 obtained as a crystalline besylate salt.

FIG. 10B This graph represents the PXRD spectrum of Compound 1 obtained as an amorphous besylate salt.

FIG. 11A This graph represents the PXRD spectrum of Compound 1 obtained as a crystalline besylate salt form 1.

FIG. 11B This graph represents the PXRD spectrum of Compound 1 obtained as a crystalline besylate salt form 2.

FIG. 11C This graph represents the PXRD spectrum of Compound 1 obtained as a crystalline besylate salt form 3.

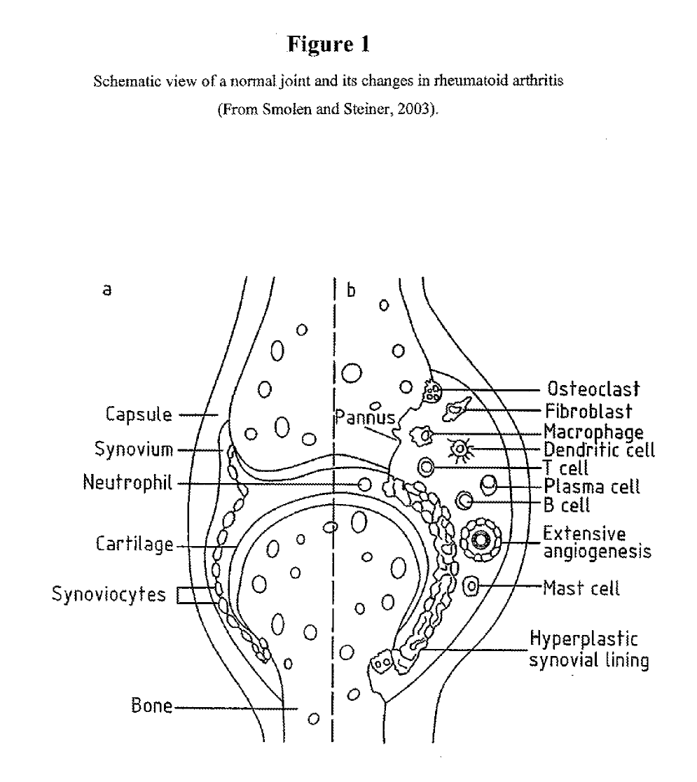

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Bioavailability’ of a drug refers to the fraction of a dose administered via a route other than intravenous injection (by definition, when a medication is administered intravenously, its bioavailability is 100%) that reaches the systemic circulation. The absolute bioavailability compares the bioavailability (estimated as the area under the curve, or AUC) of the active drug in systemic circulation following non-intravenous administration (e.g., after oral, rectal, transdermal, subcutaneous, or sublingual administration), with the bioavailability of the same drug following intravenous administration. It is the fraction of the drug absorbed through non-intravenous administration compared with the corresponding intravenous administration of the same drug. The comparison must be dose-normalized if different doses are used; consequently, each AUC is corrected by dividing the corresponding dose administered.

‘Pharmaceutically acceptable vehicle’ refers to a diluent, adjuvant, excipient or carrier with which a compound of the invention is administered.

‘Solvate’ refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Therapeutically effective amount’ means the amount of a compound of the invention that, when administered to a subject for treating a disease, is sufficient to effect such treatment for the disease. The “therapeutically effective amount” can vary depending on the compound, the disease and its severity, and the age, weight, etc., of the subject to be treated.

‘Preventing’ or ‘prevention’ refers to a reduction in risk of acquiring or developing a disease or disorder (i.e., causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein the term ‘compound(s) of the invention’, and equivalent expressions, are meant to embrace compounds according to Formula I, IIa or IIb as herein described, which expression includes the pharmaceutically acceptable salts, the solvates of the compounds, and the solvates of the pharmaceutically acceptable salts, e.g., hydrates, where the context so permits. Similarly, reference to intermediates, whether or not they themselves are claimed, is meant to embrace their salts, and solvates, where the context so permits.

As used herein the term ‘salt(s) of the invention’, and equivalent expressions, are meant to embrace pharmaceutically acceptable salts of the compound(s) according to Formula I, IIa or IIb as herein described, which expression includes the solvates of the pharmaceutically acceptable salts, e.g., hydrates, where the context so permits.

As used herein the term ‘salt forming agent’ refers to a substance with which a compound of the invention is to form a salt. Example of salt forming agents include adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, or toluenesulfonic acid.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of a compound of the invention provided herein, radioactive or not, are intended to be encompassed within the scope of the invention.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Such tautomers, as appropriate, are encompassed within the compounds of the invention as disclosed herein.

Tautomeric forms may be relevant to the attainment of the optimal chemical reactivity and biological activity of a compound of interest.

A compound of the invention may possess one or more asymmetric centers; such a compound can therefore be produced as an individual (R)- or (S)-stereoisomer or as a mixture thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compounds

The present invention is based on the discovery that MAPKAPK5 functions in the pathway that results in the expression of MMP1, and that inhibitors of MAPKAPK5 activity, such as the compounds of the invention, are useful for the treatment of diseases involving the abnormally high expression of MMP activity.

The salts of the invention may be described generally as salts of a [1.2.4]triazolo[1,5-a]pyrazine, substituted in the 5-position by a 3-amido-furan-4-yl, and an in the 8-position by a substituted 4-piperazino-4-aniline group.

More particularly, the present invention relates to salts of the compound according to Formula I:

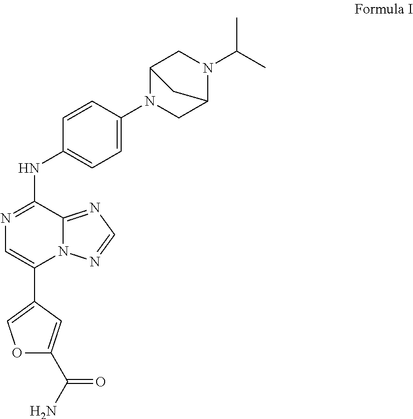

wherein the said salt is a salt formed with adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, or toluenesulfonic acid.

In a particular embodiment, the present invention relates to salts of the compound according to Formula IIa:

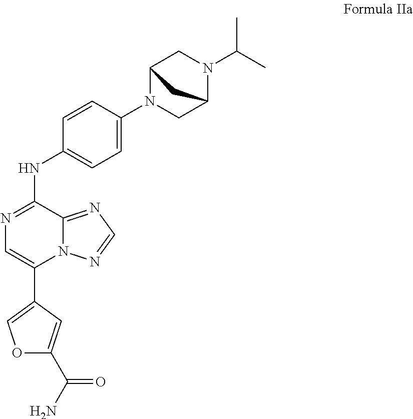

wherein said salt is a salt formed with adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, or toluenesulfonic acid.

In a another particular embodiment, the present invention relates to salts of the compound according to Formula IIb:

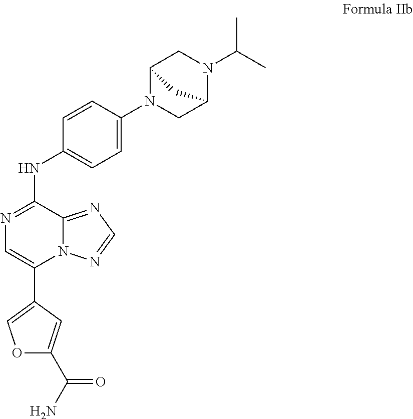

wherein said salt is a salt formed with adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, or toluenesulfonic acid.

In another more particular embodiment, the salt of the invention according to Formula I, IIa or IIb is the salt formed with benzenesulfonic acid, naphthalene-1,5-disulfonic acid or toluene sulfonic acid. In an even more particular embodiment, the salt of the invention according to Formula I, IIa or IIb is the salt formed with benzenesulfonic acid.

In a particular embodiment, the salt of the invention according to Formula I, IIa or IIb is the salt formed with adipic acid.

In a particular embodiment, the salt of the invention according to Formula I, IIa or IIb is the salt formed with L-aspartic acid.

In a particular embodiment, the salt of the invention according to Formula I, IIa or IIb is the salt formed with benzenesulfonic acid.

In a particular embodiment, the salt of the invention according to Formula I, IIa or IIb is the salt formed with benzoic acid.

In a particular embodiment, the salt of the invention according to Formula I, IIa or IIb is the salt formed with caprylic acid.

In a particular embodiment, the salt of the invention according to Formula I, IIa or IIb is the salt formed with citric acid.

In a particular embodiment, the salt of the invention according to Formula I, IIa or IIb is the salt formed with fumaric acid.

In a particular embodiment, the salt of the invention according to Formula I, IIa or IIb is the salt formed with gentisic acid.

In a particular embodiment, the salt of the invention according to Formula I, IIa or IIb is the salt formed with L-glutamic acid.

In a particular embodiment, the salt of the invention according to Formula I, IIa or IIb is the salt formed with glycolic acid.

In a particular embodiment, the salt of the invention according to Formula I, IIa or IIb is the salt with hydrochloric acid.

In a particular embodiment, the salt of the invention according to Formula I, IIa or IIb is the salt formed with L-lactic acid.

In a particular embodiment, the salt of the invention according to Formula I, IIa or IIb is the salt formed with L-malic acid.

In a particular embodiment, the salt of the invention according to Formula I, IIa or IIb is the salt formed with maleic acid.

In a particular embodiment, the salt of the invention according to Formula I, IIa or IIb is the salt formed with L-mandelic acid.

In a particular embodiment, the salt of the invention according to Formula I, IIa or IIb is the salt formed with methanesulfonic acid.

In a particular embodiment, the salt of the invention according to Formula I, IIa or IIb is the salt formed with naphthalene-1,5-disulfonic acid.

In a particular embodiment, the salt of the invention according to Formula I, IIa or IIb is the salt formed with 1-hydroxy-2-naphthoic acid.

In a particular embodiment, the salt of the invention according to Formula I, IIa or IIb is the salt formed with phosphoric acid.

In a particular embodiment, the salt of the invention according to Formula I, IIa or IIb is the salt formed with saccharin,

In a particular embodiment, the salt of the invention according to Formula I, IIa or IIb is the salt formed with succinic acid.

In a particular embodiment, the salt of the invention according to Formula I, IIa or IIb is the salt formed with sulfuric acid.

In a particular embodiment, the salt of the invention according to Formula I, IIa or IIb is the salt formed with L-tartaric acid.

In a particular embodiment, the salt of the invention according to Formula I, IIa or IIb is the salt formed with toluenesulfonic acid.

In another aspect, the present invention provides an adipate, L-aspartate, benzenesulfonate, benzoate, caprylate, citrate, fumarate, gentisate, L-glutamate, glycolate, hydrochloride, L-lactate, L-malate, maleate, L-mandelate, mesylate, naphthalene-1,5-disulfonate, 1-hydroxy-2-naphthoate, phosphorate, saccharate, succinate, sulfate, L-tartarate, or toluenesulfonate salt of the compound according to Formula I, IIa, or IIb.

In another aspect, the present invention provides an adipate, L-aspartate, besylate, benzoate, caprylate, citrate, fumarate, gentisate, L-glutamate, glycolate, hydrochloride, L-lactate, L-malate, maleate, L-mandelate, methanesulfonate, napadisylate, xinafoate, phosphate, saccharinate, succinate, sulfate, L-tartrate, or tosylate salt of the compound according to Formula I, IIa, or IIb.

In one embodiment, with respective the salt of compound according to Formula I, IIa or IIb, the salt is a benzoate, citrate, fumarate or mesylate salt. In an even more particular embodiment, with respective the salt of compound according to Formula I, IIa or IIb, the salt is fumarate.

In another embodiment, with respective the salt of compound according to Formula I, IIa or IIb, the salt is a benzenesulfonate (besylate), naphthalene-1,5-disulfonate (napadisilate) or toluene sulfonate (tosylate) salt. In an even more particular embodiment, with respective the salt of compound according to Formula I, IIa or IIb, the salt is benzenesulfonate (besylate).

In another aspect, the salt of the invention is a 1:1 free base/salt forming agent adduct.

In a further aspect, the present invention provides pharmaceutical compositions comprising a salt of the invention, and a pharmaceutical carrier, excipient or diluent. In this aspect of the invention, the pharmaceutical composition can comprise one or more of the salts of the invention described herein. Moreover, the salts of the invention useful in the pharmaceutical compositions and treatment methods disclosed herein, are all pharmaceutically acceptable as prepared and used.

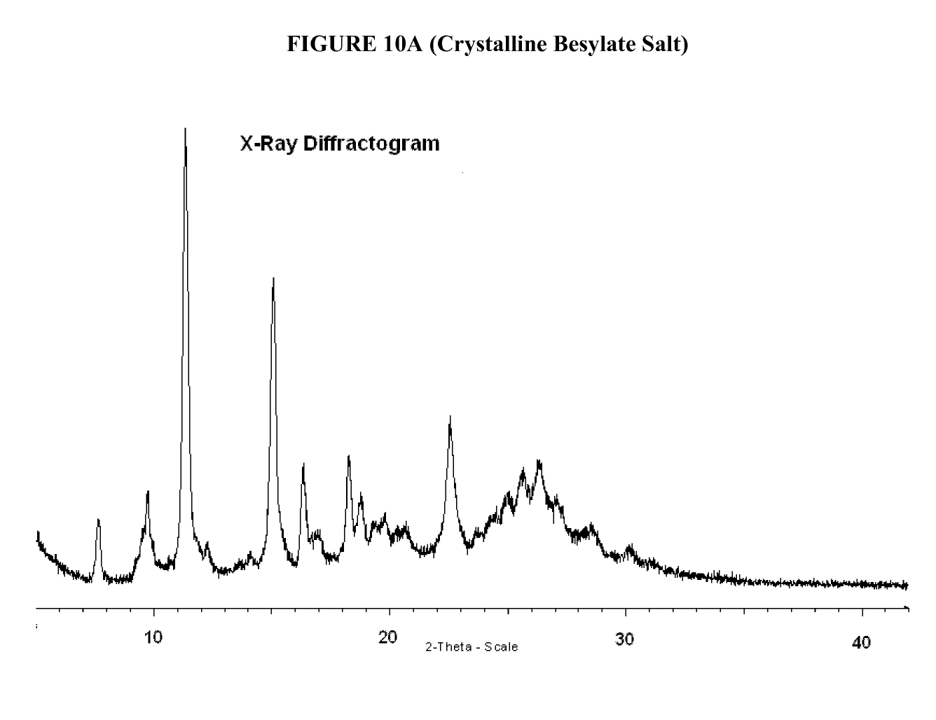

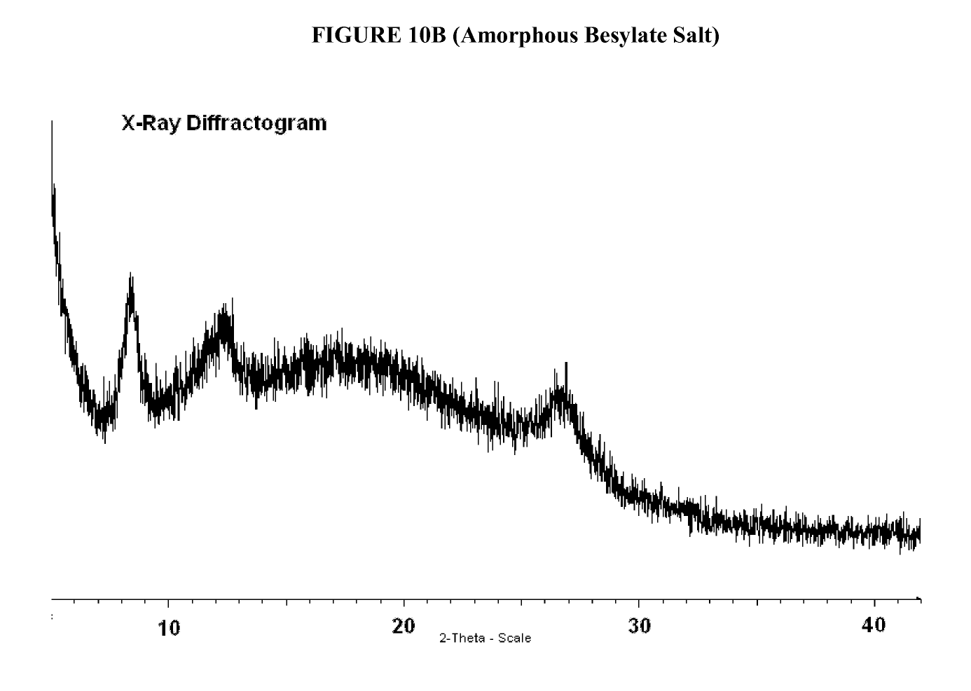

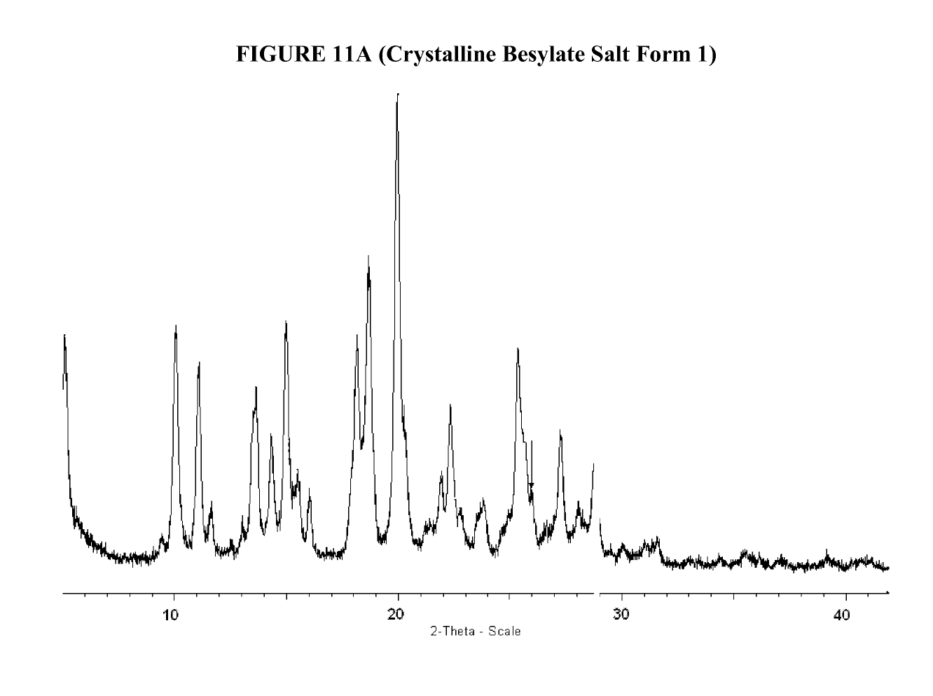

In a further embodiment, the salt of the invention is in crystalline form. In a particular embodiment, the salt of the invention is characterized by the PXRD pattern expressed in terms of 2 theta angles as shown on FIG. 10A.

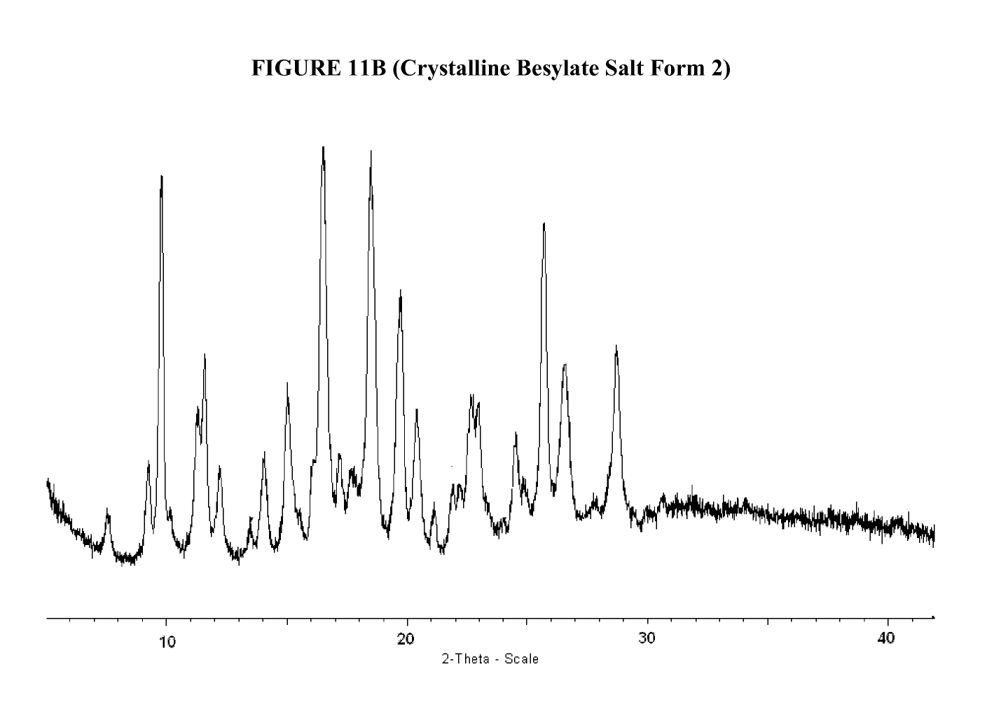

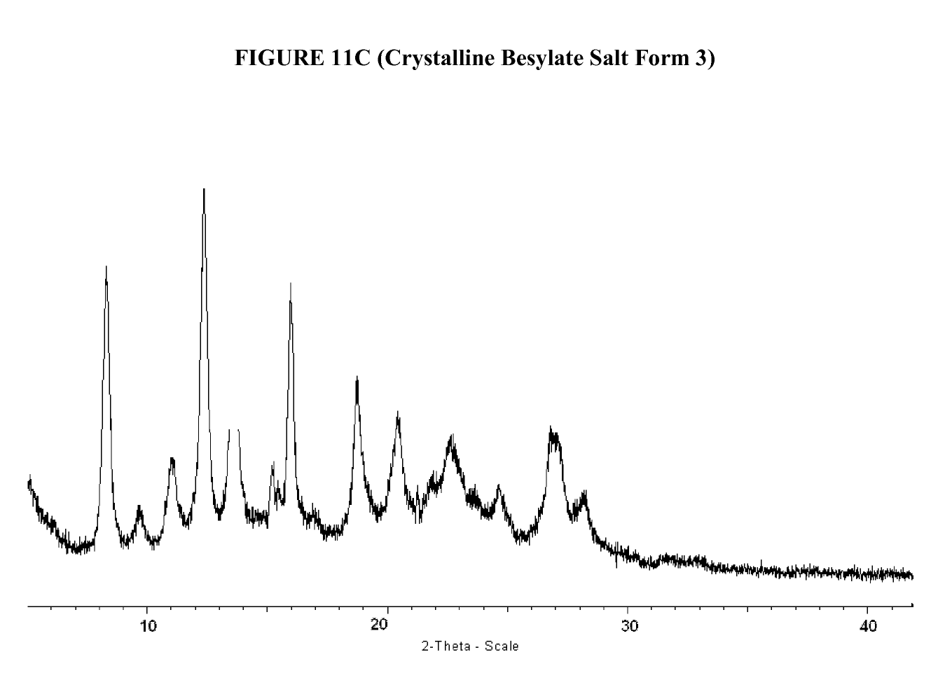

In another further embodiment, the salt of the invention is in crystalline form. In a particular embodiment, the salt of the invention is characterized by the PXRD pattern expressed in terms of 2 theta angles) (2θ/°), in at least 7 positions selected from the group consisting of: 5.1, 9.4, 10.0, 11.1, 11.6, 13.0, 13.6, 14.3, 14.9, 15.5, 16.0, 18.2, 18.6, 20.0, 21.2, 21.9, 22.3, 22.8, 23.8, 25.4, 25.7, 26.0, 27.3, 28.1, 28.8, 31.1, and 31.6, as shown on FIG. 11A and Table A. In further particular embodiment the salt of the invention contains at least 10, at least 12, at least 15 or at least 20 of the 2θ/° listed above. In a further particular embodiment the salt of the invention contains all of the 2θ/° listed above.

In another further embodiment, the salt of the invention is in crystalline form. In a particular embodiment, the salt of the invention is characterized by the PXRD pattern expressed in terms of 2 theta)angles (2θ/°), in at least 7 positions selected from the group consisting of: 7.5, 9.3, 9.7, 11.2, 11.6, 12.2, 13.5, 14.0, 15.0, 16.1, 16.5, 17.2, 17.7, 18.5, 19.7, 20.4, 21.1, 21.9, 22.2, 22.6, 22.9, 24.0, 24.5, 24.8, 25.7, 26.6, and 28.8, as shown on FIG. 11B and Table B. In further particular embodiment the salt of the invention contains at least 10, at least 12, at least 15 or at least 20 of the 2θ/° listed above. In a further particular embodiment the salt of the invention contains all of the 2θ/° listed above.

In a further embodiment, the salt of the invention is in crystalline form. In a particular embodiment, the salt of the invention is characterized by the PXRD pattern expressed in terms of 2 theta angles,) (2θ/°), in at least 7 positions selected from the group consisting of: 8.3, 9.7, 11.0, 12.3, 13.6, 15.2, 16.0, 18.7, 20.4, 21.2, 21.8, 22.6, 24.6, 26.9, and 28.2 as shown on FIG. 11C and Table C. In further particular embodiment the salt of the invention contains at least 10, at least 12, at least 15 or at least 20 of the 2θ/° listed above. In a further particular embodiment the salt of the invention contains all of the 2θ/° listed above.

In one embodiment, the salts of the invention are obtained by combining a compound of Formula I, IIa, or IIb together with an acid selected from adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, and toluenesulfonic acid, in an inert solvent and precipitating said salt from said solvent. In a particular embodiment, the salt of the invention is obtained by adding the compound of Formula I and a salt forming agent in a suitable solvent in order to achieve full dissolution, followed by a controlled solvent evaporation in order to achieve supersaturation, and thus crystallization of the corresponding salt.

In one embodiment, the salt of the invention is obtained by mixing a compound of Formula I, IIa, or IIb and an acid are combined in a molar ratio of between 5:1 and 1:5 of compound:acid. In a particular embodiment, the salt of the invention is obtained by mixing a compound of Formula I, IIa, or IIb and an acid are combined in a molar ratio of between 2:1 and 1:2 of compound:acid. In a more particular embodiment, the salt of the invention is obtained by mixing a compound of Formula I, IIa, or IIb and an acid are combined in a molar ratio of 1:1.

In another particular embodiment, the solvent for the preparation of the salt of the invention is selected from DMSO, acetone, THF, MTBE, dioxane, EtOAc, MeOH/DCM, or toluene. In a more particular embodiment, the solvent is selected from DMSO and MeOH/DCM.

In another particular embodiment, the solvent for the preparation of the salt of the invention is selected from iPrOH/water, iPrOH, iBuOH, or tBuOH. In a more particular embodiment, the solvent is iPrOH/water.

In another aspect the invention provides a process for preparing the salt of any of Formulae I, IIa or IIb or the salts thereof as recited, comprising the steps of
i) reacting the compound of Formula I, IIa or IIb with an acid selected from adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, and toluenesulfonic acid, in an inert solvent; and
ii) precipitating the said salt from the said solvent.

In one embodiment of the invention, with respect to the preparation of the salt, the acid is selected from benzoic acid, citric acid, fumaric acid and methanesulfonic acid.

In one particular embodiment of the invention, with respect to the preparation of the salt, the acid is fumaric acid.

In another particular embodiment of the invention, with respect to the preparation of the salt, the acid is benzenesulfonic acid, naphthalene-1,5-disulfonic acid or toluene sulfonic acid.

In a more particular embodiment of the invention, with respect to the preparation of the salt, the acid is benzenesulfonic acid.

In a further aspect of the invention there is also provided a salt of a compound of Formula I, IIa or IIb obtainable by or obtained by the aforementioned process.

In one embodiment of the invention, with respect to the preparation of the salt, the said compound of Formula I, IIa or IIb and the said acid are reacted in a molar ratio of between 5:1 and 1:5.

In one embodiment of the invention, with respect to the preparation of the salt, the said compound of Formula I, IIa or IIb and the said acid are reacted in a molar ratio of between 2:1 and 1:2.

In one embodiment of the invention, with respect to the preparation of the salt, the said compound of Formula I, IIa or IIb and the said acid are reacted in a molar ratio of 1:1.

In one embodiment of the invention, with respect to the preparation of the salt, the inert solvent is selected from DMSO, acetone, THF, MTBE, dioxane, EtOAc, MeOH/DCM, or toluene.

In one particular embodiment of the invention, with respect to the preparation of the salt, the inert solvent is selected from DMSO and MeOH/DCM.

In one embodiment, with respect to the inert solvent MeOH/DCM, the ratio MeOH/DCM ranges from 3/1 to 1/3. In a particular embodiment, the ratio MeOH/DCM is 1/3.

In another particular embodiment, with respect to the preparation of the salt, the inert solvent is selected from iPrOH/water, iPrOH, iBuOH, and tBuOH.

In another particular embodiment, with respect to the preparation of the salt, the inert solvent is selected from iPrOH, and tBuOH.

In a another particular embodiment, with respect to the preparation of the salt, the inert solvent is selected from iPrOH/water. In a more particular embodiment, with respect to the inert solvent iPrOH/water, the ratio iPrOH/water ranges from 3/1 to 9/1. In another particular embodiment, the ratio iPrOH/water is 3/1.

In one aspect a salt of the invention according to any one of the embodiments herein described is a solvate of a salt of the invention.

### Pharmaceutical Compositions

When employed as pharmaceuticals, a salt of the invention is typically administered in the form of a pharmaceutical composition. Such compositions can be prepared in a manner well known in the pharmaceutical art and comprise at least one active ingredient.

Generally, a salt of the invention is administered in a pharmaceutically effective amount. The amount of the salt actually administered will typically be determined by a physician, in the light of the relevant circumstances, including the condition to be treated, the chosen route of administration, the actual compound -administered, the age, weight, and response of the individual patient, the severity of the patient's symptoms, and the like.

The pharmaceutical compositions of this invention can be administered by a variety of routes including oral, rectal, transdermal, subcutaneous, intravenous, intramuscular, and intranasal. Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A salt of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

A salt of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1

### Tablets

A salt of the invention may be admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

### Formulation 2

### Capsules

A salt of the invention may be admixed as a dry powder with a starch diluent in an approximate 1:1 weight ratio. The mixture is filled into 250 mg capsules (125 mg of active compound per capsule).

### Formulation 3

### Liquid

A salt of the invention (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then be added to produce a total volume of 5 mL.

### Formulation 4

### Tablets

A salt of the invention may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5

### Injection

A salt of the invention may be dissolved or suspended in a buffered sterile saline injectable aqueous medium to a concentration of approximately 5 mg/mL.

### Formulation 6

### Topical

Stearyl alcohol (250 g) and a white petrolatum (250 g) may be melted at about 75° C. and then a mixture of a salt of the invention (50 g) methylparaben (0.25 g), propylparaben (0.15 g), sodium lauryl sulfate (10 g), and propylene glycol (120 g) dissolved in water (about 370 g) may be added and the resulting mixture would be stirred until it congeals.

### Methods of Treatment

A salt of the invention may be used as a therapeutic agent for the treatment of conditions in mammals that are causally related or attributable to aberrant activity of MMP1 and/or MAPKAPK5. Accordingly, the salts of the invention and pharmaceutical compositions thereof find use as therapeutics for preventing and/or treating inflammatory diseases in mammals including humans. Thus, and as stated earlier, the present invention includes within its scope, and extends to, the recited methods of treatment, as well as to the salts for use in such methods, and for the preparation of medicaments useful for such methods.

In a first aspect, the present invention provides a salt of the invention for use in medicine.

In a method of treatment aspect, this invention provides a method of treating a mammal susceptible to or afflicted with a condition associated with extra-cellular matrix (ECM) degradation, in particular arthritis, and more particularly, rheumatoid arthritis which method comprises administering an effective amount of a salt of the invention or a pharmaceutical composition thereof.

In another method of treatment aspect, the invention provides a method of treating a mammal susceptible to or afflicted with a condition associated with an abnormal cellular expression of MMP1, which comprises administering a therapeutically effective amount of a salt of the invention, or a pharmaceutical composition thereof.

In another method of treatment aspect, the present invention provides a method of treatment or prophylaxis of a condition characterized by abnormal matrix metallo proteinase activity, which comprises administering a therapeutically effective matrix metallo proteinase inhibiting amount of a salt of the invention, or pharmaceutical composition thereof.

In yet another method of treatment aspect, this invention provides methods of treating a mammal susceptible to or afflicted with diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; and renal disorders method comprises administering an effective condition-treating or condition-preventing amount of a salt of the invention or pharmaceutical compositions thereof.

This invention also relates to the use of a salt of the invention in the manufacture of a medicament for treatment or prophylaxis of a condition prevented, ameliorated or eliminated by administration of an inhibitor of Mitogen-Activated Protein Kinase-Activated Protein Kinase 5, or a condition characterised by abnormal collagenase activity, or a condition associated with ECM degradation or a condition selected from diseases involving inflammation, most preferably in for the treatment of rheumatoid arthritis.

As a further aspect of the invention there is provided a salt of the invention for use as a pharmaceutical. In a preferred embodiment there is provided a salt of the invention for use as a pharmaceutical in the treatment or prevention of the aforementioned conditions and diseases. Also provided herein is the use of a salt of the invention in the manufacture of a medicament for the treatment or prevention of one of the aforementioned conditions and diseases.

In a further aspect the present invention provides a salt of the invention for use in the prevention or treatment of conditions in mammals that are causally related or attributable to aberrant activity of MMP1 and/or MAPKAPK5. In particular, the present invention provides a salt of the invention and/or pharmaceutical compositions thereof for use in the treatment or prevention of inflammatory diseases in mammals including humans.

In a further aspect, this invention provides a salt of the invention for use in the prevention or treatment of a condition associated with extra-cellular matrix (ECM) degradation, in particular arthritis, and more particularly, rheumatoid arthritis.

In a further aspect, this invention provides a salt of the invention for use in the prevention or treatment of a condition associated with an abnormal cellular expression of MMP1.

In a further aspect, this invention provides a salt of the invention for use in the prevention or treatment of a condition characterized by abnormal matrix metallo proteinase activity.

In a further aspect, this invention provides a salt of the invention for use in the prevention or treatment of diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; and renal disorders.

In another embodiment, this invention provides a salt of the invention for the manufacture of a medicament to treat diseases involving inflammation.

In a further embodiment, this invention provides a salt of the invention for the manufacture of a medicament to treat rheumatoid arthritis.

In a further embodiment, this invention provides a salt of the invention for the manufacture of a medicament to treat a condition characterized by ECM degradation.

A particular regimen of the present method comprises the administration to a subject in suffering from a disease condition characterized by extracellular matrix degradation, with an effective matrix metallo-protease inhibiting amount of a salt of the invention for a period of time sufficient to reduce the abnormal levels of extracellular matrix degradation in the patient, and preferably terminate, the self-perpetuating processes responsible for said degradation. A special embodiment of the method comprises administering of an effective matrix metallo-protease inhibiting amount of a salt of the present invention to a subject patient suffering from or susceptible to the development of rheumatoid arthritis, for a period of time sufficient to reduce or prevent, respectively, collagen and bone degradation in the joints of said patient, and preferably terminate, the self-perpetuating processes responsible for said degradation.

The compounds of the invention may show high crystallinity, improved processability, improved chemical stability, low hygroscopy, lower dissolution energy, better absorption, less toxicity, good absorption, good half-life, good solubility, low protein binding affinity, less drug-drug interaction, and good metabolic stability. In a particular aspect, the compounds of the present invention exhibit unexpected significant improvements in pharmacological properties over similar compounds, in particular they may exhibit improved efficacy, improved stability, improved solubility, improved processability and improved tolerability, which improvements are also reflected in its salt forms. In at least some embodiments, the salts of the present invention are expected to exhibit unexpected significant improvements in chemical stability and/or solubility over the free base. Where the compounds of the invention exhibit any one or more of these improvements, this may have an effect on their use, or the use of the salts of the invention in the conditions described herein. For example, where the compounds of the invention exhibit an improved efficacy it would be expected that the compounds or salts of the invention could be administered at a lower dose, thus reducing the occurrence of any possible undesired side effects. Similarly, where the compounds of the invention exhibit increased tolerability, this might allow the compounds of the invention to be dosed at a higher concentration without causing unwanted side effects. Such alterations in efficacy or tolerability might be expected to result in an improved therapeutic window for said compounds of the invention, which improved therapeutic window is equally found in the salts of the invention. Similarly, improvements in the other properties listed above will also confer advantages in the potential uses of the compounds of the invention.

The salts of the invention may show improved solubility over the free base form of the respective compounds of the invention, thus allowing easier formulation compared to the less soluble compound of the invention. In turn, a better formulation form may lead to better patient compliance, due to an easier administration route, thus facilitating the treatment of the above mentioned conditions. For example, oral formulation may be particular by the patient when compared for example and without limitation, to injection, or infusion.

The salts of the invention may also show an improved chemical and/or physical stability, thus leading to an extended shelf-life of the compound.

It will also be appreciated by a person of skill in the art, that the salts of the invention may offer an improved processability and manufacturability, when compared to the corresponding compounds of the invention in their free base form. This improvement may result from the improved properties of the salts herein described including higher crystallinity and low hygroscopy.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as inflammatory and autoimmune conditions, the regimen for treatment usually extends over many months or years, and accordingly oral dosing is particular for patient convenience and tolerance. With oral dosing, one to five and especially two to four and typically three oral doses per day are representative regimens. Using these dosing patterns, each dose provides from about 0.01 to about 20 mg/kg of the salt of the invention, with particular doses each providing from about 0.1 to about 10 mg/kg and especially about 1 to about 5 mg/kg.

Transdermal doses are generally selected to provide similar or lower blood levels than are achieved using injection doses.

When used to prevent the onset of an inflammatory condition, the salts of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A salt of the invention can be administered as the sole active agent or it can be administered in combination with other therapeutic agents, including other salts that demonstrate the same or a similar therapeutic activity, and that are determined to safe and efficacious for such combined administration. In a specific embodiment, co-administration of two (or more) agents allows for significantly lower doses of each to be used, thereby reducing the side effects seen.

In one embodiment, a salt of the invention is co-administered with another therapeutic agent for the treatment and/or prevention of a disease involving inflammation; particular agents include, but are not limited to, immunoregulatory agents e.g. azathioprine, corticosteroids, cyclophosphamide, cyclosporin A, FK506, Mycophenolate Mofetil, OKT-3 and ATG.

In one embodiment, a salt of the invention is co-administered with another therapeutic agent for the treatment and/or prevention of rheumatoid arthritis; particular agents include but are not limited to analgesics, non-steroidal anti-inflammatory drugs (NSAIDS), steroids, synthetic DMARDS (for example but without limitation methotrexate, leflunomide, sulfasalazine, auranofin, sodium aurothiomalate, penicillamine, chloroquine, hydroxychloroquine, azathioprine, and ciclosporin), and biological DMARDS (for example but without limitation Infliximab, Etanercept, Adalimumab, Rituximab, and Abatacept).

By co-administration is included any means of delivering two or more therapeutic-agents to the patient as part of the same treatment regime, as will be apparent to the skilled person. Whilst the two or more agents may be administered simultaneously in a single formulation this is not essential. The agents may be administered in different formulations and at different times.

### General Synthetic Procedures

The compounds of the invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following methods are presented with details as to the preparation of representative bicycloheteroaryls that have been listed hereinabove. The compounds of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm) ¹H NMR spectra were recorded on a Bruker DPX 400 NMR spectrometer (400 MHz). Chemical shifts (δ) for ¹H NMR spectra are reported in parts per million (ppm) relative to tetramethylsilane (δ 0.00) or the appropriate residual solvent peak, i.e. CHCl₃ (δ 7.27), as internal reference. Multiplicities are given as singlet (s), doublet (d), triplet (t), quartet (q), multiplet (m) and broad (br). Coupling constants (J) are given in Hz. Electrospray MS spectra were obtained on a Micromass platform LC/MS spectrometer. Column Used for all LCMS analysis: Waters Acquity UPLC BEH C18 1.7 μm, 2.1 mm ID×50 mm L (Part No. 186002350)). Preparative HPLC: Waters XBridge Prep C18 5 μm ODB 19 mm ID×100 mm L (Part No. 186002978). All the methods are using MeCN/H₂O gradients. H₂O contains either 0.1% TFA or 0.1% NH₃.

List of abbreviations used in the experimental section

| | | | |
|---|---|---|---|
| DCM: | Dichloromethane | LC-MS | Liquid Chromatography-Mass |
| DiPEA: | N,N-diisopropylethylamine | | Spectrometry |
| MeCN | Acetonitrile | Ppm | part-per-million |
| BOC | tert-Butyloxy-carbonyl | EtOAc | ethyl acetate |
| DMF | N,N-dimethylformamide | APCI | atmospheric pressure chemical |
| TFA | Trifluoroacetic acid | | ionization |
| THF | Tetrahydrofuran | Rt | retention time |
| NMR | Nuclear Magnetic Resonnance | RT | Room Temperature |
| DMSO | Dimethylsulfoxide | s | singlet |
| DPPA | Diphenylphosphorylazide | br s | broad singlet |
| m | multiplet | PVDF | Polyvinylidene Fluoride |
| d | doublet | RIPA buffer | Radioimmunoprecipitation assay |
| PdCl₂dppf | [1,1′- | | buffer |
| | Bis(diphenylphosphino)ferrocene]dichloropalladium(II) | MAPKAPK5 | Mitogen-activated protein kinase- |
| TEA | Triethylamine | | activated protein kinase 5 |
| AIBN | 2,2′-azobisisobutyronitrile | PBMC | Peripheral Blood Mononuclear |
| IPA | Iso-Propyl Alcohol | | Cell |
| BINAP | 2,2′-bis(diphenylphosphino)-1,1′- | TNFα | Tumor Necrosis Factor alpha |
| | binaphthyl | LPS | Lipopolysaccharide |
| MTBE | Methyl tent-Butyl Ether | ip | Intra-peritoneal |
| 2-MeTHF | 2-Methyl Tetrahydrofuran | iv | Intraveinous |
| EDTA | Ethylenediaminetetraacetic acid | RH | Relative Humidity |
| ATP | Adenosine triiphosphate | DSC | Differential scanning calorimetry |
| EGTA | Ethylene Glycol Tetraacetic Acid | DVS | Dynamic vapor sorption |
| BSA | Bovine Serum Albumine | TG-FTIR | Thermogravimetry coupled with |
| DTT | Dithiothreitol | | Fourier Transformed infrared |
| FBS | Fetal bovine serum | | spectroscopy |
| PBST | Phosphate buffered saline with | PXRD | Powder X-Ray Diffraction |
| | Tween 3.2 mM Na₂HPO₄, 0.5 mM | | |
| | KH₂PO₄, 1.3 mM KCl, 135 mM | | |
| | NaCl, 0.05% Tween 20, pH | | |
| | 7.4 | | |
| MMP | Matrix Metallo Proteinase | | |
| shRNA | short hairpin RNA | | |
| RNA | Ribonucleic acid | | |
| Ad-Si RNA | Adenoviral encoded siRNA | | |
| DMEM | Dulbecco's Modified Eagle | | |
| | Medium | | |
| APMA | 4-aminophenylmercuric acetate | | |
| hCAR | human cellular adenovirus | | |
| | receptor | | |
| dNTP | deoxyribonucleoside triphosphate | | |
| QPCR | quantitative polymerase chain | | |
| | reaction | | |
| cDNA | copy deoxyribonucleic acid | | |
| GAPDH | Glyceraldehyde phosphate | | |
| | dehydrogenase | | |

### Synthetic Preparation of Compounds of the Invention

### Example 1

### Synthesis of Intermediates

### Intermediate 1a: Preparation of 3,6-Dibromo-pyrazin-2-ylamine

### General Reaction Scheme:

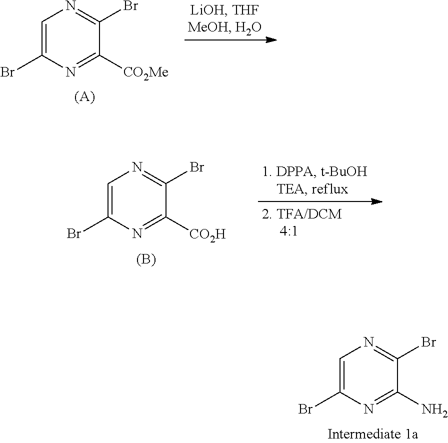

### Step 1: Synthesis of compound (B) as described in the general reaction scheme; 3,6-dibromo-pyrazine-2-carboxylic acid

LiOH (655 mg, 27 mmol) is added to a solution of 3,6-dibromo-pyrazine-2-carboxylic acid methyl ester (A) (J. Med. Chem. 1969, 12, 285-87) (2.7 g, 9 mmol) in THF:water:MeOH (18:4.5:4.5 mL). The reaction is stirred at 5° C. for 30 min, concentrated in vacuo, taken up in DCM and washed with 1N HCl. The organic phase is dried over anhydrous MgSO₄ and concentrated in vacuo to afford compound (B). ¹H NMR (250 MHz, CDCl₃) δ ppm 8.70 (s, 1H).

### Step 2: Synthesis of Intermediate 1a as described in the general reaction scheme; 3,6-Dibromo-pyrazin-2-ylamine

Diphenylphosphorylazide (2.59 mL, 12 mmol) and triethylamine (1.67 mL, 12 mmol) are added to a solution of 3,6-dibromo-pyrazin-2-carboxylic acid (3.52 g, 12 mmol) in t-butanol (90 mL). The reaction is heated at reflux for 18 hours. The reaction is quenched with water, then concentrated in vacuo and taken up in DCM. The organic solution is washed with water and 1N NaOH, dried over anhydrous MgSO₄ and concentrated in vacuo. The resultant solid is filtered through a pad of silica using EtOAc, then concentrated and TFA:DCM (4:1, 12 mL) is added to the solid and stirred for 30 min. The solution is concentrated in vacuo then neutralised with 1N NaOH and extracted with DCM. The organic layer is dried over anhydrous MgSO₄ and concentrated in vacuo to give the product. ¹H NMR (250 MHz, DMSO-d6) [symbol] ppm 7.25 (br s, 2H), 7.68 (s, 1H); m/z (APCI) 254 (M+H)⁺; m.p 135-139° C.

### Alternative Route to Intermediate 1a

Alternatively, Intermediate 1a can also be obtained using the following synthetic sequence:

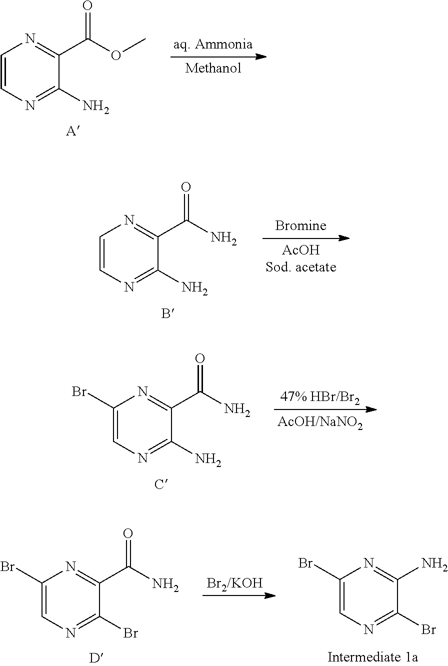

### Step 1: Synthesis of Intermediate B′ as described in the general scheme: 2-amino-3-amidopyrazine

A mixture of A′ (270 g, 1.763 M), methanol (1.05 L) and liq. Ammonia (25%) (1.68 L) is stirred at 30° C. for 24 hrs. The resulting solid is separated by filtration, washed with water (210 mL×2) and dried at 50-55° C. under vacuum to afford B′, used in the next step without further treatment.

### Step 2: Synthesis of Intermediate C′ as described in the general scheme: 2-amino-3-amido-5-bromopyrazine

A mixture of B′ (210 g, 1.52 mM), acetic acid (630 mL) and sodium acetate tri-hydrate (310 gm, 2.28 mM) is warmed up to 50° C. To this solution is added bromine (93.43 mL, 1824 mM) in acetic acid (105 mL) in 1.5 hrs and the mixture is stirred for 30 min at 50-55° C. The reaction mixture is cooled to 15° C., water (2.1 L) is added over 20-30 min and the resulting suspension is stirred for another 30 min. The Solid is isolated by filtration, washed with water (210 mL×2) and dried at 45° C. under vacuum to afford C′, used in the next step without further treatment.

### Step 3: Synthesis of Intermediate D′ as described in the general scheme: 2,5-dibromo-3-amidopyrazine

A mixture of C′ (200 g, 922 mM), aq. hydrobromic acid 47% (500 mL) and acetic acid (100 mL) is cooled to −10° C. Bromine (47.25 mL, 922 mM) is then added to the reaction mixture over 45 min at −10° C. A solution of sodium nitrite (190 g, 2.765 M) in water (400 mL) is then added over 2h, whilst maintaining the reaction mixture temperature below −5° C. Upon completion of the addition, the reaction mixture is further stirred for 30 min. A solution of sodium thiosulfate (250 g) in water (712 mL) is then added over 30 min forming a suspension. The solid is isolated by filtration, washed with water (400 mL×2) and dried at 50-55° C. under vacuum to afford crude D′. Crude D′ (186 g) is suspended in a solution of 2-butanone (93 mL) and hexane (279 mL) and then stirred at 30° C. for 1 hr. The solid is isolated by filtration, the cake is washed with a 1:3 mixture of 2-butanone (93 mL) and hexane (186 mL) and dried at 50° C. for 3 hrs to afford clean D′.

### Step 4: Synthesis of Intermediate 1a

To a stirred solution of potassium hydroxide (130 g, 2.314 M) in water (1.3 L) at −5° C. is added bromine (23.7 mL, 463 mM) over 0.5-1 h. Intermediate D′ (100 g, 356 mM) is added to the reaction mixture and stirred for 15 min at −5° C. The temperature of the reaction mixture is raised to 15° C. over 20 min and stirred at this temperature for an additional 30 min. The resulting solid is separated by filtration, washed with water (50 mL×3) and dried at 50-55° C. under vacuum to afford crude Intermediate 1a.

Intermediate 1a (51 g) is heated in a 10% aq. solution of potassium hydroxide (510 mL) at 70° C. for 2 hrs, then cooled to 30° C., and the solid is filtered. The cake is washed with water (25 mL×2) and dried to afford clean Intermediate 1a.

### Intermediate 1b: Preparation of 3-Chloro-6-bromo-pyrazin-2-yl-amine

Alternatively 3-chloro-6-bromopyrazin-2-yl-amine can be used in place of 3,6-dibromo-pyrazin-2-yl amine and is prepared according to the following scheme:

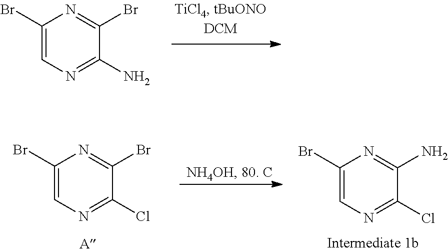

### Step 1: Synthesis of compound (A″) as described in the general reaction scheme; 2-chloro-3,5-dibromo-pyrazine

To a well stirred solution of 2-amino-3,5-dibromopyrazine (3.21 g, 12.692 mmol) in DCM (20 mL) cooled to 0° C. is added TiCl₄ (2.41 g, 12.692 mmol, 1.00 equiv.) in one portion, thus giving a dark red slurry. t-Butylnitrite (2.62 g, 25.385 mmol, 2.00 equiv.) is then added dropwise, causing the solution to turn bright yellow. The ice bath is then removed and the reaction is then allowed to proceed at room temperature. More TiCl₄ (1.50 g, 1.2 equiv.) is added and the mixture is stirred further for one hour. At that point an orange solution has formed and LC-MS shows full conversion of the starting material to the desired product which ionises very poorly. Water (100 mL) is added to the reaction, forming an emulsion. DCM (50 mL) is added, and the DCM layer is separated, and the aqueous layer is further extracted with DCM (3×50 mL) until the DCM layer is colorless. The DCM layers are gathered, washed with brine and dried over anhydrous Na₂SO₄, to yield after solvent removal, compound A″ (2.81 g, 82%) as an orange oil, which is used as such in the following step.

### Step 2: Synthesis of Intermediate 1b as described in the general reaction scheme; 3-chloro-6-bromopyrazin-2-yl amine

Compound A″ described in the previous step (9.5 g, 37.55 mmol) is suspended in concentrated NH₄OH (60 mL) and the resulting mixture is heated in a pressure autoclave to 80° C., typically overnight. The vessel is then allowed to cool down to room temperature slowly, and is then further cooled in an ice bath, causing the precipitation of the desired material. The solid is separated by filtration, washed with cyclohexane, to afford after drying, the title Intermediate 1b (5 g) as a 83/17 mixture of regiosiomers. The mixture is then purified by column chromatography. M+H+, m/z=209

### Intermediate 2: 5,8-Dibromo-1,2,41-triazolo[1,5-a]pyrazine

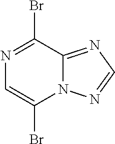

### General Scheme:

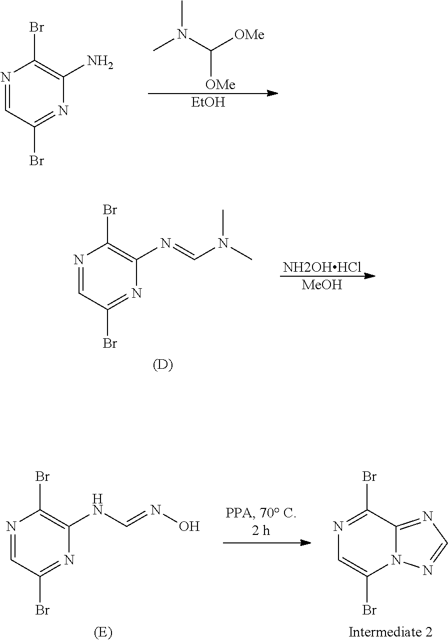

### Step 1: N′-(3,6-Dibromo-pyrazin-2-yl)-N,N-dimethylformamidine (D)

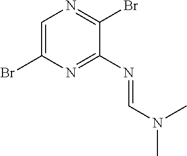

A mixture of 3,6-dibromo-pyrazin-2-ylamine (15.37 g, 60.80 mmol) and N,N-dimethylformamide dimethyl acetal (10.1 mL, 76.00 mmol), suspended in ethanol (150 mL), is refluxed for 2 hours. The reaction mixture is evaporated in vacuo affording the title compound. ¹H-NMR (400 MHz, CDCl₃) δ(ppm) 3.20 (s, 3H), 3.21 (s, 3H), 7.93 (s, 1H), 8.48 (s, 1H). LCMS: Rt 3.81 min (99.1%), m/z (APCI) 307 (M+H)⁺.

### Step 2: N-(3,6-Dibromo-pyrazin-2-yl)-N′-hydroxyformamidine (E)

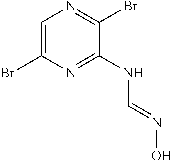

To a solution of N-(3,6-dibromo-pyrazin-2-yl)-N,N-dimethylformamidine (18.6 g, 60.80 mmol) in methanol (200 mL) is added hydroxylamine hydrochloride (5.91 g, 85.12 mmol) in one portion. The reaction is stirred at room temperature for 16 hours. The solvent is evaporated and the solid residue is treated with cold (ice cooling) water and collected by filtration. The precipitate is washed twice with water and petroleum ether and dried in vacuo yielding the title compound. ¹H-NMR (400 MHz, DMSO-d6) δ(ppm) 7.82 (br s, 1H), 8.21 (s, 1H), 8.34 (m, 1H), 11.17 (br s, 1H). LCMS: Rt 3.17 min (98.7%), m/z (APCI) 295 (M+H)⁺.

Alternatively, N-(3,6-Dibromo-pyrazin-2-yl)-N′-hydroxyformamidine (E) can be obtained directly from Intermediate 1a (100 gm, 395.3 mM), solubilised in iso-propanol (180 mL) at 80° C., to which DMF.DMA (188.4 gm, 1581 mM) is added. The reaction mixture is cooled to 30° C. and hydroxyl amine hydrochloride (49.45 gm, 711.5 mM) is added. The reaction mixture is again heated at 50° C. for 1.5 hrs, then cooled to 30° C. and water (200 mL) is added. Mixture is stirred further, and the resulting solid is filtered, washed with water (100 mL×3) and dried at 30° C. under vacuum to afford crude (E).

Crude (E) (102 gm) is re-suspended in iso-propanol (1632 mL) and heated at 80° C. for 0.5-1 hr to get clear solution. Mixture is brought to 30° C. and stirred at this temperature for 12 hrs. The resulting solid is filtered, washed with iso-propanol (500 mL×2) and dried to afford clean (E).

### Step 3: 5,8-Dibromo-[1,2,4]triazolo[1,5-a]pyrazine (Intermediate 2)

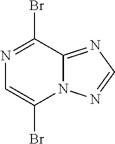

N-(3,6-dibromo-pyrazin-2-yl)-N-hydroxyformamidine (17.4 mg, 58.80 mmol) is treated with polyphosphoric acid (150 g) for one hour at 50° C. and then for 1.75 hours at 70° C. After cooling to room temperature, water is added to the reaction mixture. The resultant suspension is brought to pH 8 by careful addition of solid NaHCO₃ in small portions. The precipitate formed is collected by filtration, washed once with 1N NaOH, three times with water and dried in vacuo. The residue is partitioned between ethyl acetate and 1N NaOH and the organic phase is washed one more time with 1N NaOH and once with brine. The organic phase is dried over anhydrous MgSO₄, filtered and evaporated to give the title compound (10.15 g) as a white solid. ¹H-NMR (400 MHz, DMSO-d6) δ (ppm) 8.43 (s, 1H), 8.92 (s, 1H). LCMS: Rt 2.73 min (94.2%), m/z (APCI) 277 (M+H)⁺.

### Alternative Protocol for Step 3

N-(3,6-dibromo-pyrazin-2-yl)-N-hydroxyformamidine (150 gm, 506.8 mM) is added to PPA (195 gm) in 1,4-dioxane (450 mL) at 45° C. Temperature of the reaction mixture is raised to 80° C. and maintained for 30 min. The reaction mixture is cooled to 40° C., quenched with ice-water and extracted with 2-methyl THF (1500 mL×3). The combined organic phase is washed with 1M sodium hydroxide solution (1500 mL×3), brine solution (1500 mL), dried with sodium sulfate and concentrated to give crude Intermediate 2, which is then re-suspended and stirred in di-isopropyl ether (225 mL). The solid is separated by filtration, and finally, the cake is washed with di-isopropyl ether (37.5 mL×2) to afford clean Intermediate 2.

### Intermediate 3: 4-(4,4,5,5-Tetramethyl-[1,3,2]dioxaborolan-2-yl)-furan-2-carboxylic acid amide

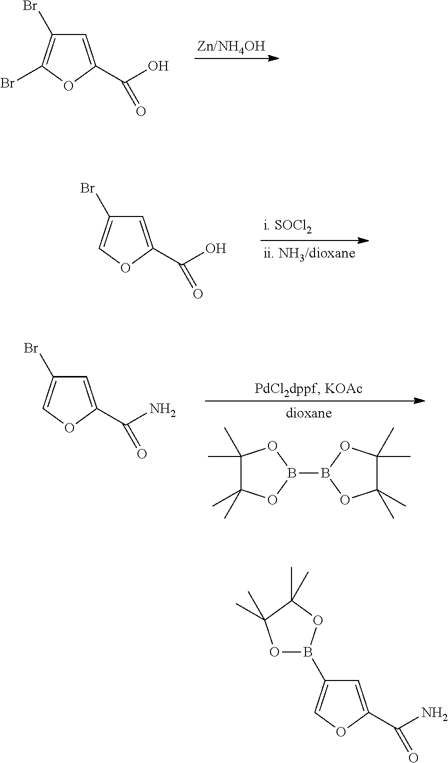

### Step 1: 4-Bromo-furan-2-carboxylic acid amide

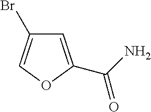

To a cooled (using a cold water bath) solution of 4,5-dibromo-furan-2-carboxylic acid (12.5 g, 46.32 mmol) in NH₄OH (100 mL) is added zinc dust [activated, powdered (washed with 2M HCl, water, MeOH, DCM) 4.54 g, 65.39 mmol] in small portions. The reaction mixture is stirred at room temperature for 10 minutes then filtered over celite and washed with water. The filtrate is cooled to −10° C. (ice/salt bath) and acidified slowly to pH 1 using conc. HCl. The aq layer is immediately extracted with ethyl acetate (4×). The organic phase is washed with brine, dried over anhydrous MgSO₄, filtrated and concentrated in vacuo to give an oil (4.96 g) which solidifies on standing to give a white solid, which is used without further purification.

The solid (4.93 g, 25.81 mmol) is dissolved in thionyl chloride (44.2 mL) and refluxed for 1 hour. After removing the solvent in vacuo the residue is dissolved in dichloromethane (75 mL) and a solution of 0.5 M NH₃ in dioxane (52 mL) is added. The reaction mixture is stirred at room temperature for 1 hour, then 33% aq. NH₃ (5 mL) is added and the reaction stirred for additional 2 hours. The solvent is removed in vacuo and the residue taken-up with a solution of sat. NaHCO₃. The basic solution is extracted using ethyl acetate (3×), the combined organic layers are dried over anhydrous MgSO₄ and concentrated in vacuo. Purification by silica gel column chromatography eluting with a mixture of (50:49:1) ethyl acetate: petroleum ether: acetic acid, affords the title compound (1.2 g, 22%).

### Step 2: 4-(4,4,5,5-Tetramethyl-[1,3,2]dioxaborolan-2-yl)-furan-2-carboxylic acid amide (Intermediate 3)

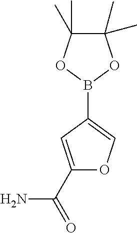

4-Bromo-furan-2-carboxylic acid amide (1.2 g, 6.32 mmol), bis(pinacolato)diboron (1.76 g, 6.94 mmol), PdCl₂.dppf (0.154 g, 0.189 mmol) and KOAc (1.85 g, 18.94 mmol) are suspended in dioxane (20 mL), purged with nitrogen for 5 minutes and then heated at 85° C. overnight. The solvent is removed in vacuo and the residue partitioned between ethyl acetate and brine. The aqueous layer is extracted four times with ethyl acetate, filtered through anhydrous MgSO₄ and evaporated. The solid residue is triturated with hexane and dried in vacuo to afford the title compound as a solid (0.984 g, 66%). N.B. compound is usually 50-60% pure by ¹H-NMR.

### Alternative Route to Intermediate 3:

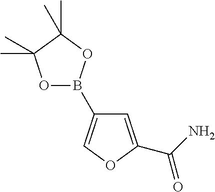

3-(4,4,5,5-Tetramethyl-[1,3,2]dioxaborolan-2-yl)-furan (5.0 g, 25.77 mmol) is dissolved in dry acetonitrile (30 mL). Chlorosulfonylisocyanate (5.47 g, 38.65 mmol, 1.5 equiv.) in solution in dry acetonitrile (20 mL) is added in one portion at room temperature to the furan producing a pink solution that subsides overnight to turn yellow. The resulting solution is cooled with an ice bath and water (5 mL) is added, causing an exotherm. The resulting mixture is partitioned between DCM (100 mL) and water (30 mL). The aqueous layer is extracted 3 more times with DCM (50 mL), then the organic layers are gathered, washed with brine (10 mL), dried over anhydrous Na₂SO₄ and finally the solvent is removed under vacuum. The oily residue is dissolved in DCM (3 mL), sonicated to give a suspension of a crystalline solid. The solid is separated by filtration, and the cake is washed with a very small amount of DCM, then diethyl ether and dried under suction to afford 3 g of the title compound as a white powder. NMR ¹H (400 MHz, DMSO-d6) δ ppm: 8.16 (1H, m); 7.15 (1H, m); 7.11 (1H, s); 4.33 (2H, q); 1.38 (3H, t); 1.34 (12H, s).

### Intermediate 4: 4-((1S,4S)-5-Isopropyl-2,5-diaza-bicyclo[2.2.1]hept-2-yl)-phenylamine

### Step 1: ((1S,4S)-5-(4-Nitro-phenyl)-2,5-diaza-bicyclo[2.2.1]heptane-2-carboxylic acid tert-butyl ester

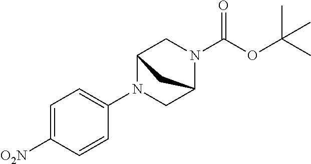

4-Fluoronitrobenzene (4.00 g, 28.348 mmol), DiPEA (5.89 mL, 60.667 mmol, 2.14 equiv.) and (1S,4S)-2-BOC-2,5-diazabicyclo[2.2.1]heptane (6.02 g, 30.333 mmol, 1.07 equiv.) are mixed in acetonitrile (20 mL). The resulting solution is heated to reflux overnight, after which full conversion has occurred. The solvent is removed under vacuum, and the solid yellow residue is stirred in cyclohexane (50 mL) for 0.25 h, then allowed to settle, the supernatant is discarded, and the process is repeated twice. On the third time, the solid is separated by filtration, allowed to dry under suction, to afford the title compound clean as a yellow solid (8.8 g).

### Step 2: (1S,4S)-2-(4-Nitro-phenyl)-2,5-diaza-bicyclo[2.2.1]heptane

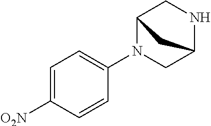

The solid obtained in the previous step (8.2 g) is dissolved in a mixture of DCM (12 mL) and TFA (12 mL). The reaction is allowed to proceed at RT for 2 h, at which point full deprotection has occurred. The volatiles are removed under vacuum and the crude resulting solid is used as such without further treatment.

### Step 3: (1S,4S)-2-Isopropyl-5-(4-nitro-phenyl)-2,5-diaza-bicyclo[2.2.1]heptane (Intermediate 4)

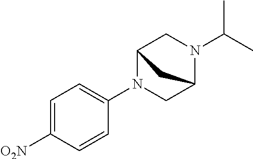

The crude compound obtained in the previous step (6.22 g, 28.356 mmol) is dissolved in acetonitrile (70 mL). K₂CO₃ (19.59 g, 141.770 mmol, 5.00 equiv.) is added, followed by i-propyl iodide (9.64 g, 56.708 mmol, 2.00 equiv.) and the resulting suspension is heated to reflux with stirring, for 3 h, at which point full conversion has occurred. The reaction mixture is partitioned between DCM (100 mL) and water (50 mL). The organic layer is washed with water (50 mL), brine (25 mL), dried on anhydrous Na₂SO₄, filtered and evaporated in vacuo to yield the title compound (9.90 g) as a yellow solid.

### Step 4: 4-(1S,4S)-5-Isopropyl-2,5-diaza-bicyclo[2.2.1]hept-2-yl)-phenylamine

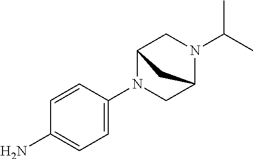

The compound obtained in the previous step (3.30 g, 9.35 mmol) is dissolved in EtOH (107 mL). The system is degassed and placed under nitrogen. Pd/C 10% (0.50 g, 5 mol %) is added followed by hydrazine, 35% in water (4.3 mL, 46.75 mmol, 5 equiv.), and the reaction is allowed to proceed at 100° C. until full conversion has occurred (typically 1 h). The reaction is then allowed to cool down, filtered on celite and the filtrate is evaporated in vacuo to afford the title compound (2.07 g) as pink oil.

### Example 2

### Specific Examples of Compounds of the Invention

### Compound 1: 4-{8-[4-((1S,4S)-5-Isopropyl-2,5-diaza-bicyclo[2.2.1]hept-2-yl)-phenylamino]-[1,2,4]triazolo[1,5-a]pyrazin-5-yl}-furan-2-carboxamide

### Step 1: (5-Bromo-[1,2,4]triazolo[1,5-a]pyrazin-8-yl)-[4-((1S,4S)-5-isopropyl-2,5-diaza-bicyclo[2.2.1]hept-2-yl)-phenyl]-amine

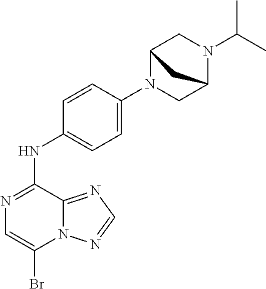

5,8-Dibromo-[1,2,4]triazolo[1,5-a]pyrazine (2.26 g, 8.14 mmol), 4-((1S,4S)-5-Isopropyl-2,5-diaza-bicyclo[2.2.1]hept-2-yl)-phenylamine (2.07 g, 8.95 mmol, 1.10 equiv.) and DiPEA (4.3 mL, 24.42 mmol, 3.00 equiv.) are mixed in isopropanol (28 mL) under nitrogen. The reaction is heated to 85° C. until completion of the reaction (typically 5 h). The solvent is removed under vacuum and the residue is partitioned between 60 mL aqueous sodium phosphates buffer (pH 7) and 200 mL DCM, the organic layer is washed with 60 mL satd. NaCl, dried on anhydrous Na₂SO₄, filtered and evaporated in vacuo to yield the title compound (3.67 g) as a green-black foamy solid.

### Step 2: 4-{8-[4-((1S,4S)-5-Isopropyl-2,5-diaza-bicyclo[2.2.1]hept-2-yl)-phenylamino]-[1,2,4]triazolo[1,5-a]pyrazin-5-yl}-furan-2-carboxamide

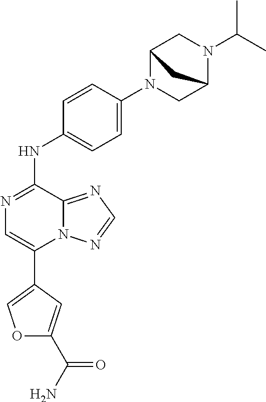

The compound obtained in the previous step (3.25 g, 7.59 mmol) is mixed with 4-(4,4,5,5-tetramethyl-[1,3,2]dioxaborolan-2-yl)-furan-2-carboxylic acid amide (2.70 g, 11.40 mmol, 1.50 equiv.), PdCld₂dppf.DCM (0.310 g, 0.38 mmol, 5 mol %), DiPEA (2.65 mL, 15.20 mmol, 2.00 equiv.) in 1,4-dioxane (51 mL) and water (13 mL). The system is sealed, purged by vacuum/N₂ and heated to 110° C. for 6 h, at which point full conversion has occurred. The reaction mixture is diluted with DCM (60 mL) and MeOH (60 mL) and filtered on celite. The filtrate is evaporated to yield a muddy brown residue. This residue is treated with EtOH (50 mL), MeOH (25 mL) and DCM (20 mL), and evaporated to dryness, then left in vacuo at 40° C. for another 1 h to try and eliminate as much moisture and alcohols as possible. The dry residue is suspended in DCM (100 mL) and sonicated for about 1 h, to disperse all the solid bits. A suspension of fine solid is obtained. It is cooled to 0° C., filtered on Buchner, and the solid is washed with DCM (30 mL) and dried under vacuum.

The residue is treated with 1M KOH (40 mL), sonicated until the solid is well dispersed, and filtered on a sintered glass funnel Finally, the solid is dissolved in DCM (450 mL) and MeOH (50 mL), washed with a mixture of saturated aqueous NaF (250 mL), water (500 mL) and iPrOH (250 mL). The organic layer is dried on anhydrous Na₂SO₄, filtered and evaporated in vacuo to yield 4-{8-[4-((1S,4S)-5-Isopropyl-2,5-diaza-bicyclo[2.2.1]hept-2-yl)-phenylamino]-[1,2,4]triazolo[1,5-a]pyrazin-5-yl}-furan-2-carboxylic acid amide (2.34 g) as a yellow-brown solid.

¹H-NMR (400 MHz, DMSO-d6): δ 9.76 (s, 1H); 8.74 (s, 1H); 8.69 (d. 1H); 8.13 (s, 1H); 7.93 (broad s, 1H); 7.86 (d, 1H); 7.72 (d, 2H); 7.54 (broad s, 1H); 6.59 (d, 2H); 4.31 (d, 1H (iPrOH)); 4.25 (s, 1.1H); 3.78 (m, 1H (iPrOH)); 3.69 (s, 1H); 3.30 (H₂O); 3.16 (d(d), 1H); 3.00 (d(d), 1H); 2.50 (DMSO); 2.42-2.37 (m, 2H); 1.81 (s, 2H); 1.04 (d, 7H) (i-PrOH); 0.95 (2 d, 6H).

### Compound 2: 4-[8-({4-[(1R,4R)-5-isopropyl-2,5-diazabicyclo[2.2.1]hept-2-yl]phenyl}amino)[1,2,4]triazolo[1,5-a]pyrazin-5-yl]-2-furamide

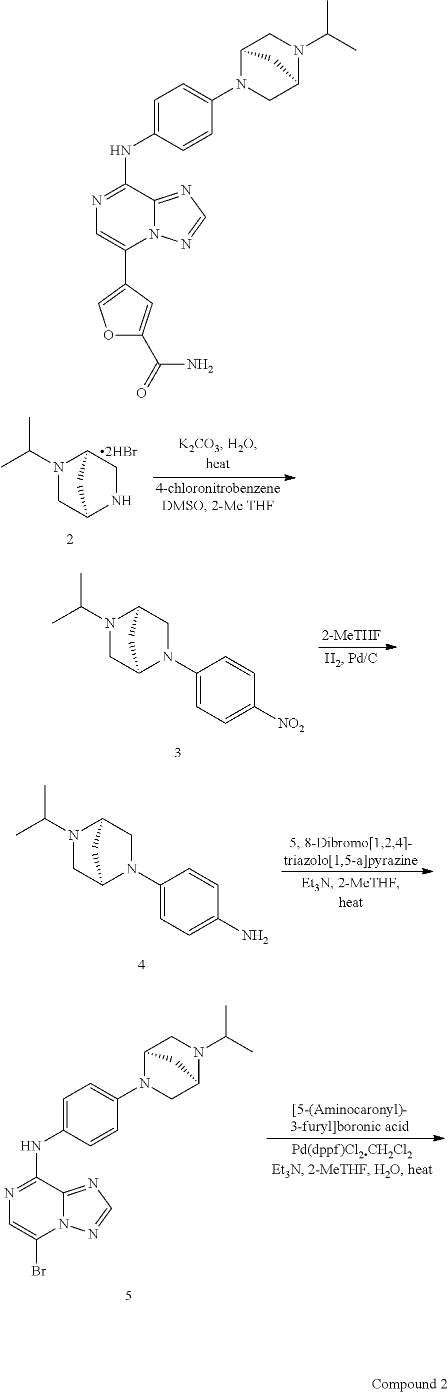

### Step 1: (1R,4R)-2-Isopropyl-5-(4-nitrophenyl)-2,5-diazabicyclo[2.2.1]heptane (3)

To a mixture of (1R,4R)-5-isopropyl-2,5-diazabicyclo[2.2.1]heptane dihydrobromide (2); 3.0 g; 9.9 mmol), 4-chloronitrobenzene (1.7 g; 11 mmol), dimethylsulfoxide (6.2 mL), and tap water (2.5 mL) is added solid K₂CO₃ (1.7 g; mmol; gas evolution). The resulting suspension is heated to 50° C., after which 2-MeTHF (0.5 mL) and more solid K₂CO₃ (1.9 g; mmol) are added. Reaction temperature is increased to 125° C. and the reactor contents are held at this temperature overnight. The reaction mixture is cooled down to ambient temperature, after which tap water (25 mL) is added. Extraction of the aqueous mixture with ethyl acetate (3×30 mL) and concentration of the combined organic extracts in vacuo give a solid residue, which is redissolved in hot MTBE (300 mL). The hot solution is filtered to remove residual solids and concentrated in vacuo to furnish crude (2). Purification is accomplished by partitioning the crude material between ethyl acetate (70 mL) and dilute hydrochloric acid (pH 1; 250 mL), separating the layers, washing of the aqueous phase with ethyl acetate (2×50 and 2×100 mL), extraction of the combined organic layers with dilute hydrochloric acid (pH 1; 100 mL), basification of the combined aqueous layers with 10 N aqueous NaOH to pH 10, extraction of the alkaline aqueous layer with MTBE (2×200 mL) and ethyl acetate (2×200 mL), drying over Na₂SO₄, and concentration in vacuo to give a white solid. This solid is reslurried in heptane/MTBE 1:1 v/v (20 mL), the resulting suspension filtered and the filter cake washed with heptane/MTBE 1:1 v/v (20 mL) and air dried to give (3) as a white solid.

LC-purity: 99.3 area-%.

### Step 2: 4-[(1R,4R)-5-Isopropyl-2,5-diazabicyclo[2.2.1]hept-2-yl]aniline (4)

A solution of 3 (1.6 g; 6.1 mmol) in 2-MeTHF (25 mL) is stirred under a 1 bar hydrogen atmosphere in the presence of Pd/C catalyst (10% Degussa type E101 NE/W; 0.1 g) at 30° C. for a period of 3 h. The catalyst is filtered off over a bed of Dicalite 478 and the filter cake washed with 2-MeTHF (2×10 mL). The filtrate is concentrated in vacuo to a volume of 25 mL and the resulting solution is used as such in the next step.

### Step 3: 5-Bromo-N-{4-[(1R,4R)-5-isopropyl-2,5-diazabicyclo[2.2.1]hept-2-yl]phenyl}[1,2,4]triazolo[1,5-a]-pyrazin-8-amine (5)

To the solution obtained from step 2 is added 5,8-dibromo[1,2,4]triazolo[1,5-a]-pyrazine (1.6 g; 5.8 mmol) and triethylamine (3.4 mL). The resulting mixture is heated at reflux for 70 h, after which the reactor contents are cooled down and filtered to remove solids. The filter cake is washed with 2-MeTHF (2×5 mL) and the filtrate used as such in the next step.

### Step 4: Compound 2 4-[8-({4-[(1R,4R)-5-Isopropyl-2,5-diazabicyclo[2.2.1]hept-2-yl]phenyl}amino)[1,2,4]triazolo[1,5-a]-pyrazin-5-yl]-2-furamide

To the solution obtained from step 3 is added 2-MeTHF (5 mL), tap water (6.5 mL), [5-(aminocarbonyl)-3-furyl]boronic acid (1.3 g; 8.6 mmol), and Pd(dppf)₂Cl₂ (0.26 g; 0.3 mmol). The resulting mixture is degassed 5 times by means of a vacuum/nitrogen purge cycle and heated at 80° C. for 6 h. The reactor contents are then cooled down to ambient temperature, 1,2-diamino-propane (2 mL) is added, the resulting suspension filtered (slow!), and the filter cake washed with 2-MeTHF (6×5 mL) to obtain crude compound 2 as a green solid. The crude material is reslurried in methanol (15 mL), filtered, and the filter cake washed with methanol (5 mL). The filter cake is then mixed with water/acetic acid (pH 1; approx. 50 mL), the resulting suspension filtered until a clear filtrate is obtained, and the filter cake washed with water until the washing liquid turned colorless. The combined filtrate and washing liquids are concentrated in vacuo at 50° C. to remove water, the residue stripped with 2-propanol (twice) and toluene (three times) and subsequently taken up in methanol (70 mL) and toluene (5 mL). To the resulting suspension is added 1,2-diaminopropane (2 mL) and dppe (0.08 g; 0 2 mmol), after which stirring is continued overnight. The purified product is isolated by filtration, washing of the filter cake with methanol until the washing liquid turns pale yellow, followed by washing with ethyl acetate, and dried at 40° C. in vacuo to compound 2 as a yellow solid.

LC-purity 98.6 area-%.
LC-MS: m/z=459 (100) [M+H]+.

TABLE 1
Examples of compounds of the invention.

| Cpd # | Structure | Name | MW | M + H+, m/z |
|---|---|---|---|---|
| 1 | 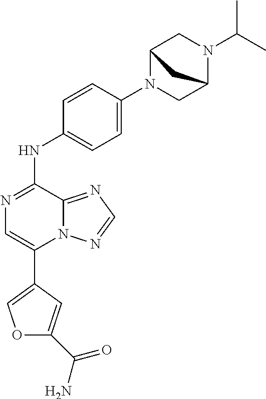 | 4-(8-(4-((1S,4S)-5-isopropyl- 2,5- diazabicyclo[2.2.1]heptan-2- yl)phenylamino)- [1,2,4]triazolo[1,5-a]pyrazin- 5-yl)furan-2-carboxamide | 458 | 459 |
| 2 | 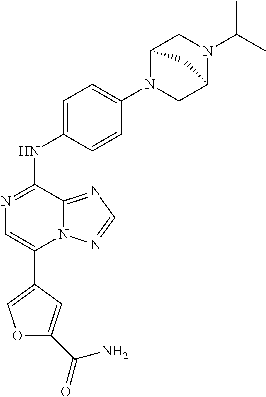 | 4-{8-[4-((1R,4R)-5- Isopropyl-2,5-diaza- bicyclo[2.2.1]hept-2-yl)- phenylamino]- [1,2,4]triazolo[1,5-a]pyrazin- 5-yl}-furan-2-carboxylic acid amide | 458 | 459 |

### Example 3

### Salt Form Preparation and Screening

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

All reagents are of commercial grade and are used as received without further purification, unless otherwise stated. Commercially available anhydrous solvents are used for reactions conducted under inert atmosphere.

Correspondence Between IUPAC and Trivial Salt Names:

| Acid | Salt |
|---|---|
| adipic acid | Adipate |
| L-aspartic acid | Aspartate |
| benzenesulfonic acid | Benzenesulfonate or |
| | besylate |
| benzoic acid | Benzoate |
| caprylic acid | Caprylate |
| citric acid | Citrate |
| fumaric acid | Fumarate |
| gentisic acid | Gentisate |
| L-glutamic acid | Glutamate |
| glycolic acid | Glycolate |
| hydrochloric acid | Chlorhydrate |
| L-lactic acid | Lactate |
| L-malic acid | Malate |
| maleic acid | Maleate |
| L-mandelic acid | Mandelate |
| methanesulfonic acid | Methanesulfonate or |
| | mesylate |
| naphthalene-1,5- | Napadisylate |
| disulfonic acid | |
| 1-hydroxy-2- | Nathpthoate or |
| naphthoic acid | xinafoate |
| phosphoric acid, | Phosphate |
| saccharin | Saccharinate |
| succinic acid | Succinate |
| sulfuric acid | Sulfate |
| L-tartaric acid | Tartrate |
| toluenesulfonic acid | Toluenesulfonate or |
| | tosylate |
| Ethylenesulfonic | Ethylenesulfonate or |
| acid | Esylate |
| 1,2- | Edisylate |
| ethylenedisulfonic | |
| acid | |

### 3.1 Analytical Conditions and Instrumentation.

| DSC | Perkin Elmer DSC 7. | |
|---|---|---|
| (Differential | Closed Au crucibles, heating rate: 10 or 20° C./min, range: −50° C. to 250° C., or | |
| scanning | DSC data are collected on a Mettler DSC 823e equipped with a 34 position auto- | |
| calorimetry) | sampler. The instrument is calibrated for energy and temperature using certified | |
| | indium. Typically 0.5-3 mg of each sample, in a pin-holed aluminium pan, is | |
| | heated at 10° C.min−1 from 25° C. to 300° C. A nitrogen purge at 50 ml · min −1 is | |
| | maintained over the sample. | |
| | The instrument control and data analysis software is STARe v9.10. | |
| DVS | Projekt Messtechnik SPS 11-100n multi-sample water vapor sorption analyzer. | |
| (Dynamic vapor | The sample is allowed to equilibrate at 50% relative humidity (r.h.) before starting | |
| sorption) | a pre-defined humidity program. | |
| | Program: | 2 h at 0% r.h. |
| | | 0 → 95% r.h. (5%/h) |
| | | 3 h at 95% r.h. |
| | | 95 → 0% (10%/h) |
| | | 2 h at 0% r.h. |

| | |
|---|---|
| Gravimetric Vapour | Sorption isotherms are obtained using a SMS DVS Intrinsic moisture sorption |
| Sorption (GVS) | analyser, controlled by SMS Analysis Suite software. The sample temperature is |
| | maintained at 25° C. by the instrument controls. The humidity is controlled by |
| | mixing streams of dry and wet nitrogen, with a total flow rate of 200 ml · min⁻¹. The |
| | relative humidity is measured by a calibrated Rotronic probe (dynamic range of |
| | 1.0-100% RH), located near the sample. The weight change, (mass relaxation) of |
| | the sample as a function of % RH is constantly monitored by the microbalance |
| | (accuracy ±0.005 mg). |
| NMR | The ¹H NMR spectra are recorded at 300.13 MHz on Bruker DPX300 instrument, |
| | or ¹H and ¹³C Spectra are obtained using a Varian Unity Inova 400 NMR |
| | spectrometer with a 5 mm inverse triple resonance probe operating at 400.12 MHz |
| | for proton. |
| | Samples are prepared in d⁶-DMSO, unless otherwise stated. |
| Raman Microscopy | Renishaw RM 1000. |
| | Stabilized diode laser 785-nm excitation, NIR-enhanced Peltier-cooled CCD |
| | camera as detector. Measurements are carried out with a long working distance 20x |
| | objective. Measurement range 2000-100 cm⁻¹. |
| FT-Raman | Bruker RFS100. |
| Spectroscopy | Nd:YAG 1064 nm excitation, 100 mW laser power, Ge detector, 64 scans, range |
| | 25-3500 cm⁻¹, 2 cm⁻¹ resolution. |
| TG-FTIR | Netzsch Thermo-Microbalance TG 209 with Bruker FT-IR Spectrometer Vector |
| (Thermogravimetry | 22. |
| coupled with | Al crucible (open or with microhole), N₂ atmosphere, heating rate 10° C. min⁻¹, |
| Fourier | range 25-250° C. |
| Transformed | |
| infrared | |
| spectroscopy) | |
| TGA | TGA data are collected on a Mettler TGA/SDTA 851e equipped with a 34 position |
| (Thermogravimetric | auto-sampler. The instrument is temperature calibrated using certified indium. |
| analysis) | Typically 5-30 mg of each sample is loaded onto a pre-weighed aluminium crucible |
| | and is heated at 10° C.min−1 from ambient temperature to 400° C. A nitrogen purge |
| | at 50 ml.min−1 is maintained over the sample. |
| | The instrument control and data analysis software is STARe v9.10. |
| Polarised Light | Samples are studied on a Leica DLM polarised light microscope with a digital |
| Microscopy (PLM) | video camera for image capture. A small amount of each sample is placed on a |
| | glass slide, mounted in immersion oil and covered with a glass slip, the individual |
| | particles being separated as well as possible. The sample is viewed with appropriate |
| | magnification and partially polarised light, coupled to a λ false-colour filter. |
| Hot Stage | Hot Stage Microscopy is carried out using a Leica DLM polarised light microscope |
| Microscopy (HSM) | combined with a Mettler-Toledo MTFP82HT hot-stage and a digital video camera |
| | for image capture A small amount of each sample is placed onto a glass slide with |
| | individual particles separated as well as possible The sample is viewed with |
| | appropriate magnification and partially polarised light, coupled to a λ false-colour |
| | filter, whilst being heated from ambient temperature typically at 6° C. min⁻¹. |
| Solubility | Suspension agitated with a temperature controlled “Thermomixer comfort” from |
| determination | Eppendorf with 800 rpm (24 hours, 23° C.). Filtered with Millipore Centrifugal |
| | Filter Device UFC30VVNB (0.1μ) and Centrifuge Hettich EBA 12 R (10000 g). |
| Thermodynamic | Aqueous solubility is determined by suspending sufficient compound in water or |
| Aqueous Solubility | buffer to give a maximum final concentration of ≧1 mg · ml⁻¹ of the parent free-form |
| by HPLC | of the compound. Quantitation is made by HPLC with reference to a standard |
| | calibration curve. The solubility is calculated using the peak areas determined by |
| | integration of the peak found at the same retention time as the principal peak in the |
| | standard injection. |
| Chemical Purity | Purity analysis is performed on a Waters Acquity system equipped with a diode |
| Determination by | array detector and Micromass ZQ mass spectrometer using MassLynx software. |
| UPLC | |
| PXRD | Bruker D8; |
| (Powder X-Ray | Copper Kₐ radiation, 40 kV/40 mA; LynxEye detector, 0.02°2θ step size, 37 s |
| Diffraction) | step time. |
| | Sample preparation A: |
| | The samples are generally measured without any special treatment other than the |
| | application of slight pressure to get a flat surface. Silicon single crystal sample |
| | holder types: a) standard holder for polymorphism screening, 0.1 mm deep, less |
| | than 20 mg sample required; b) 0.5 mm deep, 12 mm cavity diameter for c. 40 mg; |
| | c) 1.0 mm deep, 12 mm cavity diameter for c. 80 mg All samples measured on the |
| | Bruker D8 are rotated during the measurement. |
| | Sample Preparation B: |
| | Samples run under ambient conditions are prepared as flat plate specimens using |
| | powder as received. Approximately 35 mg of the sample is gently packed into a |
| | cavity cut into polished, zero-background (510) silicon wafer. The sample is rotated |
| | in its own plane during analysis. |
| | Data collection: |
| | Angular range: 2 to 42; °2θ Step size: 0.05 °2θ; Collection time: 0.5 seconds per |
| | step. |
| | Bruker D2 Phaser |
| | X-Ray Powder Diffraction patterns are collected on a Bruker AXS D2 |
| | diffractometer using Cu K radiation (30 kV, 10 mA), θ-θ geometry, using a |
| | Lynxeye detector form 5-42 2θ. |
| | The software used for data collection is DIFFRAC.SUITE and the data are |
| | analysed and presented using Diffrac Plus EVA v 13.0.0.2. |
| | Data collection: |
| | Angular range: 5 to 42 °2θ; Step size: 0.012 °2θ; Collection time: 0.15 seconds per |
| | step. |
| | Sample Preparation: |
| | Samples run under ambient conditions are prepared as flat plate specimens using |
| | powder as received without grinding. Approximately 1-2 mg of the sample is |
| | lightly pressed on a silicon wafer to obtain a flat surface. |

Reagent grade solvents are used in all other cases, unless otherwise specified.

Some margin of error is present in each of the 2 theta angle assignments. The margin of error will be dependent on a number of factors, including the exact temperature at which the values are measured. The margin of error in the foregoing 2 theta angles is approximately ±0.2 degrees for each of the foregoing peak assignments.

Since some margin of error is possible in the assignment of 2 theta angles, a useful method of comparing XRPD patterns in order to identify the particular form of a sample of the compound of Formula (I) is to overlay the XRPD pattern of the one sample form of the compound of Formula (I) over the XRPD pattern of the other sample form of the compound of Formula (I).

Significant variations in the observed endotherms are expected in respect of the DSC thermogram of each of the forms of the compound of Formula (I), based on the specific instrument and pan configuration employed, the analyst's sample preparation technique, and the sample particle size and weight. Some margin of error is normally present in the endotherm characteristics, i.e. the margin of error is approximately in the order of ±2.000 C.

### 3.2 Protocol for the Salt Screening

### 3.2.1 Crystallisation Protocol

Crystallization experiments are performed in a 96-well quartz microtiter plate. Stock solutions of the free drug are prepared in DMSO, THF, 1,4-dioxane and MeOH/DCM 1:3 v/v. Stock solutions of the counterions (adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid and toluenesulfonic acid) are prepared in DMSO, THF, 1,4-dioxane and MeOH. For amino acids and mineral acids only stock solutions in water are used. Stoichiometric volumes of stock solutions of the free drug and of the selected salt forming agents are added. The solvents are evaporated under controlled N₂ flow (˜0.4 L/min). After Raman data of the obtained residues are recorded, four new solvents are added (acetone, MTBE, EtOAc and toluene) for phase equilibration (slurry). After two days the solvents are again evaporated under controlled N₂ flow (˜0.4 L/min)

At least one Raman spectrum and one image (using crossed polarizers) are collected for each residue. The spectra of the residues are compared to the spectra of the free drug and of the counterions. This combination provides clear indications for successful salt formation (differences between the Raman spectra of the free base, the salt former and the possible salt) and crystallization (sharp and strong Raman peaks, birefringence).

The solids obtained further to the quick screen, are analysed for salt formation by ¹H NMR and Raman Spectroscopy. The level of crystallinity is then ranked using light microscopy (using crossed polarizer), and the selected salts are scaled-up.

### 3.2.2 Screening Using Dioxane as a Solvent

22 lots of 50 mg of Compound 1 are treated with dioxane (5 mL, 100 volumes) and heated until fully dissolved. Acid solution (1.1 eq.) is added and the reactions are allowed to cool to RT (hydrochloric acid, naphthalene-1,5-disulfonic acid, sulfuric acid, toluenesulfonic acid, benzenesulfonic acid, saccharin, L-aspartic acid, maleic acid, phosphoric acid, L-glutamic acid, 1-hydroxy-2-naphthoic acid, gentisic acid, L-tartaric acid, citric acid, L-mandelic acid, L-malic acid, glycolic acid, L-lactic acid, benzoic acid, succinic acid, adipic acid, caprylic acid). The solids produced are filtered, dried on the filter bed and then analysed by PXRD.

### 3.2.3 Screening Using Isopropanol as a Solvent

22 lots of 50 mg of Compound 1 are treated with isopropanol (1.5 ml, 30 volumes) and warmed to 50° C. before the addition of acid solutions (hydrochloric acid, naphthalene-1,5-disulfonic acid, sulfuric acid, toluenesulfonic acid, benzenesulfonic acid, saccharin, L-aspartic acid, maleic acid, phosphoric acid, L-glutamic acid, 1-hydroxy-2-naphthoic acid, gentisic acid, L-tartaric acid, citric acid, L-mandelic acid, L-malic acid, glycolic acid, L-lactic acid, benzoic acid, succinic acid, adipic acid, caprylic acid). The reactions are then shaken at 50° C. overnight before being cooled to RT, filtered and analysed by XRPD.

### 3.2.4 Screening Using DMF

22 lots of 50 mg of Compound 1 are treated with DMF (2.5 ml, 50 volumes) and warmed until all the material has dissolved. The acid solutions (hydrochloric acid, naphthalene-1,5-disulfonic acid, sulfuric acid, toluenesulfonic acid, benzenesulfonic acid, saccharin, L-aspartic acid, maleic acid, phosphoric acid, L-glutamic acid, 1-hydroxy-2-naphthoic acid, gentisic acid, L-tartaric acid, citric acid, L-mandelic acid, L-malic acid, glycolic acid, L-lactic acid, benzoic acid, succinic acid, adipic acid, caprylic acid)are added and the reactions are allowed to cool to RT. Reactions which do not produce solids are cooled to 4° C. and if no solids are produced the experiments are warmed to 50° C. before the addition of 2 ml of TBME. The reactions are then allowed to cool to RT or 4° C.

### 3.3 Protocol for the Scale-Up of the Salt Formation.

### 3.3.1 Preparation of Benzoate Salt.

The preparation of this salt is carried out as follows: 99.3 mg of Compound 1 as a free base is dissolved in 12 mL of DCM/MeOH 3:1 (v/v). 4.33 mL of benzoic acid solution (0.05 mol/L in MeOH) is added. The solvent is evaporated at RT under gentle N₂ flow (without flow control). 2 mL of MTBE is added to the sticky residue and the mixture is stirred at RT for three days. The resulting solid is filtered off and dried in vacuum.

### 3.3.2 Preparation of the Citrate Salt.

The preparation of this salt is carried out as follows: 99.8 mg of Compound 1 as a free base is dissolved in 12 mL DCM/MeOH 3:1 (v/v). 4.35 mL citric acid solution (0.05 mol/L in MeOH) is added. The solvent is evaporated at RT under gentle N₂ flow (without flow control). 2 mL MTBE is added to the sticky residue and the mixture is stirred at RT for three days. The resulting solid is filtered off and dried in vacuum.

### 3.3.4 Preparation of the Fumarate Salt.

The preparation of this salt could be achieved via one of the following protocols:

Protocol A

100.8 mg of Compound 1 as a free base is dissolved in 4.5 mL DMSO. 4.40 mL fumaric acid solution (0.05 mol/L in DMSO) is added. The solvent is evaporated at RT under gentle N₂ flow (without flow control). Partly crystalline powder is obtained.

Protocol B

99.3 mg of Compound 1 as a free base is dissolved in 12 mL DCM/MeOH 3:1 (v/v). 4.32 mL fumaric acid solution (0.05 mol/L in MeOH) is added. Precipitation is observed. The suspension is stirred at RT for five days. The resulting solid is filtered off and dried in vacuum.

Protocol C

151.4 mg of Compound 1 as a free base is dissolved in 18 mL DCM/MeOH 3:1 (v/v). 6.6 mL fumaric acid solution (0.05 mol/L in MeOH) is added. Precipitation is observed. The suspension is stirred at RT for six days. The resulting solid is filtered off and dried in vacuum.

### 3.3.5 Preparation of the Mesylate Salt or Methanesulfonate Salt.

71 μL methanesulfonic acid is added to a mixture of 50 mL MeOH and 500 mg of Compound 1 as a free base. A clear solution is formed. The MeOH is evaporated in vacuum at 40° C. 50 mL diethyl ether is added to the residue and the mixture is stirred overnight. The resulting solid is filtered off and dried in vacuum

### 3.3.6 General Protocol for the Preparation of Sulfonate Salts.

The sulfonate salts are prepared in a similar fashion: 200 mg of Compound 1 is treated with water (100 ml) and 1 eq of the appropriate acid. The reactions are warmed to aid dissolution and, if required, co-solvent such at tBuOH is added (ca 60 ml). The reactions are then dried by lyophilisation. The material is then used without further characterisation in the maturation experiments.

For the maturation experiments ˜20 mg of the amorphous salt is treated with 1 ml of solvent (described in the table below) and placed in a maturation chamber which is cycled between ambient and 50° C. with four hours spent under each condition. After 2.5 days the samples are examined by optical microscopy for signs of crystallinity.

TABLE 2
Solvents used for maturation of the besylate and edisylate salts.

| Salt | Solvent |
|---|---|
| Besylate | 2-Propanol, or |
| | 2-Butanol |
| Edisylate | Methylethyl ketone, or |
| | 2-Propanol, or |
| | 2-Butanol, or |
| | 2-Methyl-1-Propanol |

### 3.3.7 Formation of the Crystalline Besylate Form 1

Amorphous besylate salt (50 mg) is matured either in acetone (2 mL), or acetonitrile (2 mL). The sample is placed in a maturation chamber, cycling from ambient temperature to 50° C., with four hours spent under each condition. After 4 days, the experiment is stopped, the solids are filtered, dried on filter bed and analyzed by PXRD, as represented on FIG. 11A. The major peaks are listed in Table A below:

TABLE A
Major peaks and relative intensity of Form 1 XPRD peaks.

| Angle 2 theta | Intensity % |
|---|---|
| 5.062 | 51.7 |
| 9.37 | 12.1 |
| 10.026 | 54 |
| 11.058 | 46.1 |
| 11.634 | 17.9 |
| 13.004 | 15.4 |
| 13.619 | 41 |
| 14.294 | 31.8 |
| 14.949 | 54.7 |
| 15.505 | 24.4 |
| 16.001 | 20.8 |
| 18.165 | 52.2 |
| 18.642 | 67.4 |
| 19.972 | 100 |
| 21.223 | 14.4 |
| 21.898 | 23.4 |
| 22.295 | 37.6 |
| 22.771 | 16.8 |
| 23.843 | 18.9 |
| 25.411 | 48.3 |
| 25.709 | 33.9 |
| 25.987 | 21.1 |
| 27.258 | 32.5 |
| 28.072 | 17.4 |
| 28.806 | 27.5 |
| 31.05 | 10.1 |
| 31.586 | 10.9 |

### 3.3.8 Formation of the Crystalline Besylate Form 2

Amorphous besylate salt (50 mg) is matured in 1,2-dimethoxyethane (2 mL). The sample is placed in a maturation chamber, cycling from ambient temperature to 50° C., with four hours spent under each condition. After 4 days, the experiment is stopped, the solids are filtered, dried on filter bed and analyzed by PXRD, as represented on FIG. 11B. The major peaks are listed in Table B below:

TABLE B
Major peaks and relative intensity of Form 2 XPRD peaks.

| Angle 2 theta | Intensity % |
|---|---|
| 7.484 | 23.6 |
| 9.251 | 33.6 |
| 9.727 | 93.9 |
| 11.236 | 45.1 |
| 11.593 | 54.7 |
| 12.189 | 31.7 |
| 13.46 | 20.1 |
| 14.015 | 34.1 |
| 14.968 | 48.8 |
| 16.08 | 33.8 |
| 16.477 | 100 |
| 17.172 | 34.5 |
| 17.668 | 31.6 |
| 18.482 | 98.7 |
| 19.673 | 68.3 |
| 20.408 | 44.3 |
| 21.122 | 24.6 |
| 21.916 | 28.2 |
| 22.174 | 28.5 |
| 22.591 | 47.5 |
| 22.929 | 45.7 |
| 24.041 | 20.8 |
| 24.537 | 38.4 |
| 24.835 | 30 |
| 25.728 | 83.8 |
| 26.582 | 54.4 |
| 28.765 | 57.4 |

### 3.3.9 Formation of the Crystalline Besylate Form 3

Amorphous besylate salt (50 mg) is matured in 1,4 dioxane (2 mL). The sample is placed in a maturation chamber, cycling from ambient temperature to 50° C., with four hours spent under each condition. After 4 days, the experiment is stopped, the solids are filtered, dried on filter bed and analyzed by PXRD, as represented on FIG. 11C. The major peaks are listed in Table C below:

TABLE C
Major peaks and relative intensity of Form 3 XPRD peaks.

| Angle 2 theta | Intensity % |
|---|---|
| 8.259 | 81.9 |
| 9.668 | 23.1 |
| 11.018 | 35.6 |
| 12.329 | 100 |
| 13.579 | 78.6 |
| 15.187 | 33.6 |
| 15.981 | 77.2 |
| 18.701 | 54.1 |
| 20.428 | 45.8 |
| 21.242 | 28.6 |
| 21.798 | 31.7 |
| 22.612 | 40.9 |
| 24.637 | 28.1 |
| 26.92 | 42 |
| 28.171 | 28 |

### 3.3.10 Formation of the Besylate Salt

For example, Compound 1 (4 g) is mixed with water (160 mL, 40 volumes) and benzenesulfonic acid (1.05 eq) is added. The mixture is warmed to reflux under stirring to obtain full dissolution of the compound. To obtain the crystalline amorphous salt, the mixture is allowed to cool down, thus causing the formation of a solid, which is separated by filtration, dried on the filter bed, and washed with a small amount of isopropanol. A crystalline solid is obtained as shown by PXRD (FIG. 10A).

To obtain a amorphous form, tent-Butanol is added (100 mL), and the mixture is further heated until full dissolution has occurred, and the amorphous solid is obtained by lyophilisation, as shown by PXRD (FIG. 10B).

### 3.3.11 Large Scale Formation of the Besylate Salt

Compound 1 as a free base (1 eq, 30g) is mixed with the solvent (iPrOH/water, 3/1 or 9/1; 13 vol). The bulk is heated to reflux before the acid (1.05 eq) dissolved in a small proportion of the solvent (2 vol) is added. The solution is stirred at reflux and solubilization occurs rapidly, and the resulting solution is seeded with solid obtained in the protocol above in 3.3.7, and allowed to cooled to room temperature (−10° C. to −30° C./hour). The resulting solids are isolated by filtration, and dried under vacuum (overnight at 40° C./0.5 mbar).

### Example 4

### Aqueous Solubility Determination

Each salt is suspended in water and shaken for 24 h at 22° C. and 400 rpm. The resulting suspensions are filtered (0.2-μm filter). The obtained solids are analyzed by FT Raman. The pH of the filtrate is measured, and the concentration of the free base is determined by HPLC.

TABLE 3
Aqueous Solubility of Compound 1 vs the Fumarate and the Mesylate.

| | Suspension | Measured solubility | | Solubility |
|---|---|---|---|---|
| Sample | content | (mg/mL) | Filtrate pH | improvement |
| Compound 1 as free base | 22.6 mg in | 0.07 | 6.9 | N/A |
| | 0.5 mL | | | |
| Compound 1 as fumarate | 20.7 mg in | 1.91 | 3.2 | 27 fold |
| salt | 0.5 mL | | | |
| Compound 1 as mesylate | 97.5 mg in | 126.9 | 2.6 | 1813 fold |
| salt | 0.5 mL | | | |

### Example 5

### Thermodynamic Aqueous Solubility Study

Aqueous solubility is determined by suspending sufficient compound in water or buffer to give a maximum final concentration of ≧1 mg·ml⁻¹ of the parent free-form of the compound. Quantitation is done by HPLC with reference to a standard calibration curve. The solubility is calculated using the peak areas determined by integration of the peak found at the same retention time as the principal peak in the standard injection. If there is sufficient solid remaining, the XRPD is collected.

TABLE 4
Study parameters

| Parameters | Values |
|---|---|
| Adsorption - Scan 1 (RH) | 40-90 |
| Desorption/Adsorption - Scan 2 | 90-Dry, Dry-40 |
| (RH) | |
| Intervals (% RH) | 10 |
| Number of Scans | 2 |
| Flow rate (ml · min −1) | 200 |
| Temperature (° C.) | 25 |
| Stability (° C. min −1) | 0.2 |
| Sorption Time (hours) | 6 hour time out |

TABLE 5
Dynamic salt solubility results

| Salt | Solubilisation medium | Solubility | |
|---|---|---|---|
| Free base | Buffer pH 7.4 | 0.01 | mg · mL⁻¹ |
| | Buffer pH 2.0 | 0.3 | mg · mL⁻¹ |
| | Pure water | 0.01 | mg · mL⁻¹ |
| Mesylate | Buffer pH 7.4 | 0.3 | mg · mL⁻¹ |
| | Buffer pH 2.0 | 2.6 | mg · mL⁻¹ |
| | Pure water | 2.7 | mg · mL⁻¹ |
| Besylate | Buffer pH 7.4 | 0.046 | mg · mL⁻¹ |
| | Buffer pH 2.0 | 0.33 | mg · mL⁻¹ |
| | Pure water | 1.2-1.3 | mg · mL⁻¹ |
| Phosphate | Buffer pH 7.4 | 0.071 | mg · mL⁻¹ |
| | Buffer pH 2.0 | 0.32 | mg · mL⁻¹ |

### Example 6

### Dynamic Vapor Sorption Analysis (DVS) Study

A DVS study is carried out on the mesylate and fumarate salts obtained according to the protocols above. The cycle for the relative humidity is run as follows: (50%→0%→95%→50%).

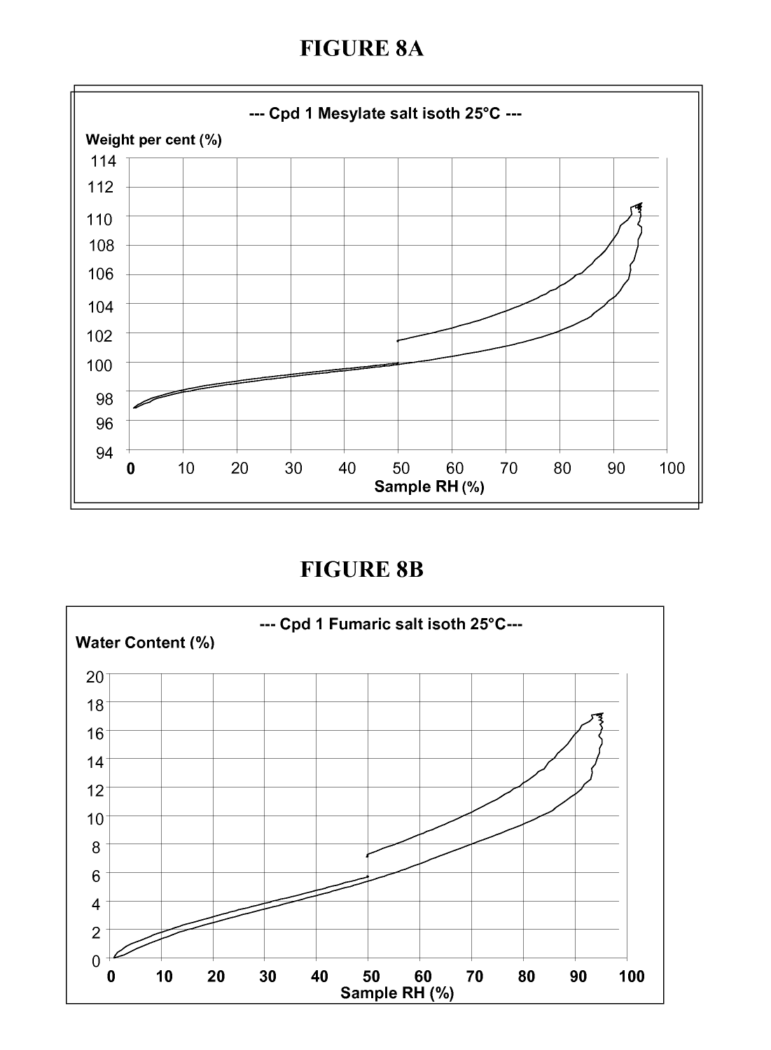

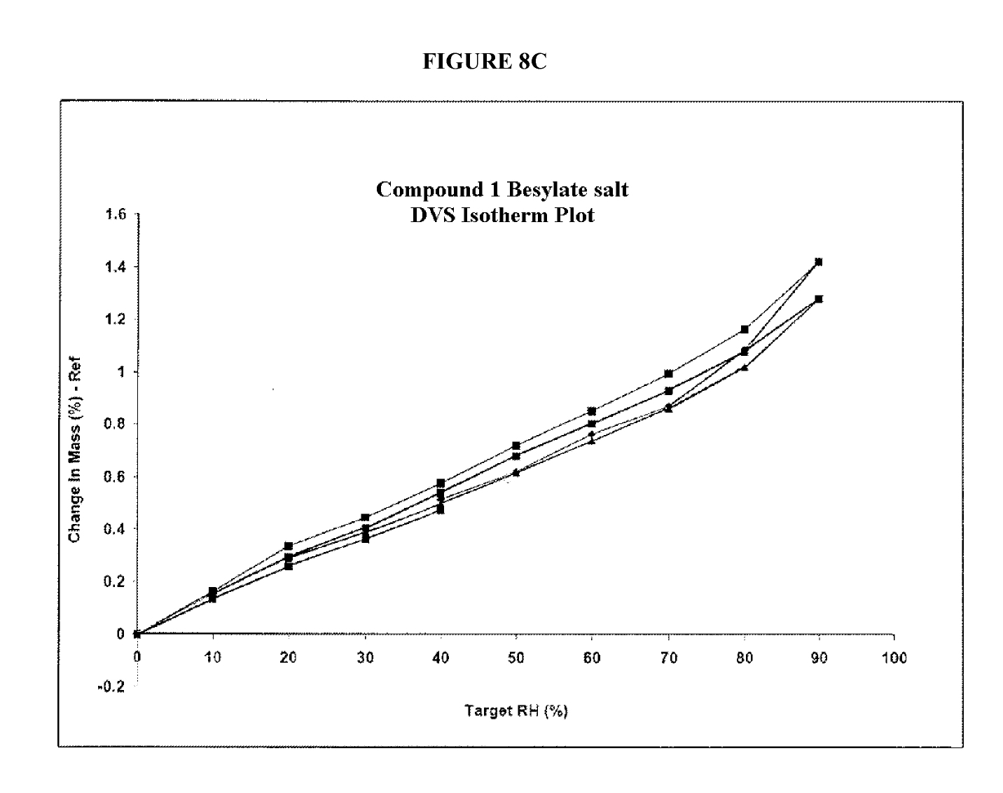

FIG. 8A shows the DVS profile of the fumarate salt indicating that the salt is weakly hygroscopic (10%), and FIG. 8B shows the DVS profile of the mesylate salt indicating that the salt is weakly hygroscopic (6%).

### Example 7

### Gravimetric Vapor Sorption Analysis (GVS) Study

Typically 5-20 mg of sample is placed in a tared mesh stainless steel basket under ambient conditions. The sample was loaded and unloaded at 40% RH and 25° C. (typical room conditions). A moisture sorption isotherm is performed as outlined below (2 scans giving 1 complete cycle). The standard isotherm is performed at 25° C. at 10% RH intervals over a 0.5-90% RH range.

The sample is recovered after completion of the isotherm and re-analysed by XRPD.

When submitted to this test, the besylate salt shows only slight hygroscopicity, taking up less than 1.45% mass on going from dryness to 90% RH. (FIG. 8C).

### Example 8

### Stability Study

### 8.1: Physical Stability Study.

The purpose of this experiment is to determine the stability of a salt of the invention vs the free base. The tested salt of the invention and the free base are used in bulk form, and the study is carried out under three conditions:

In an open container, at RT under 5% relative humidity

In an open container, at RT under 56% relative humidity

In an open container, at RT under 75% relative humidity

Samples of the salt form and of the free base are taken at four time points: t=0, t=one month, t=two months, and t=three months. Each sample is then analysed by Raman spectroscopy, DSC and PXRD analysis.

### 8.2: Chemical Stability

The purpose of this experiment is to determine the stability of a salt of the invention vs the free base. The tested salt of the invention and the free base are used in bulk form, and the study is carried out under four conditions:

In an open container, at 25° C. under 60% relative humidity

In an open container, at 40° C. under 75% relative humidity

In a closed container, at 50° C.

In a closed container, at 5° C.

Samples of the salt form and of the free base are taken at four time points: t=0, t=one month, t=two months, and t=three months. The appearance of each sample is monitored and the samples are further analysed for purity by HPLC, and the water content is measured using a Karl-Fisher analysis.

### Example 9

### Raman Spectroscopy and PXRD Characterization

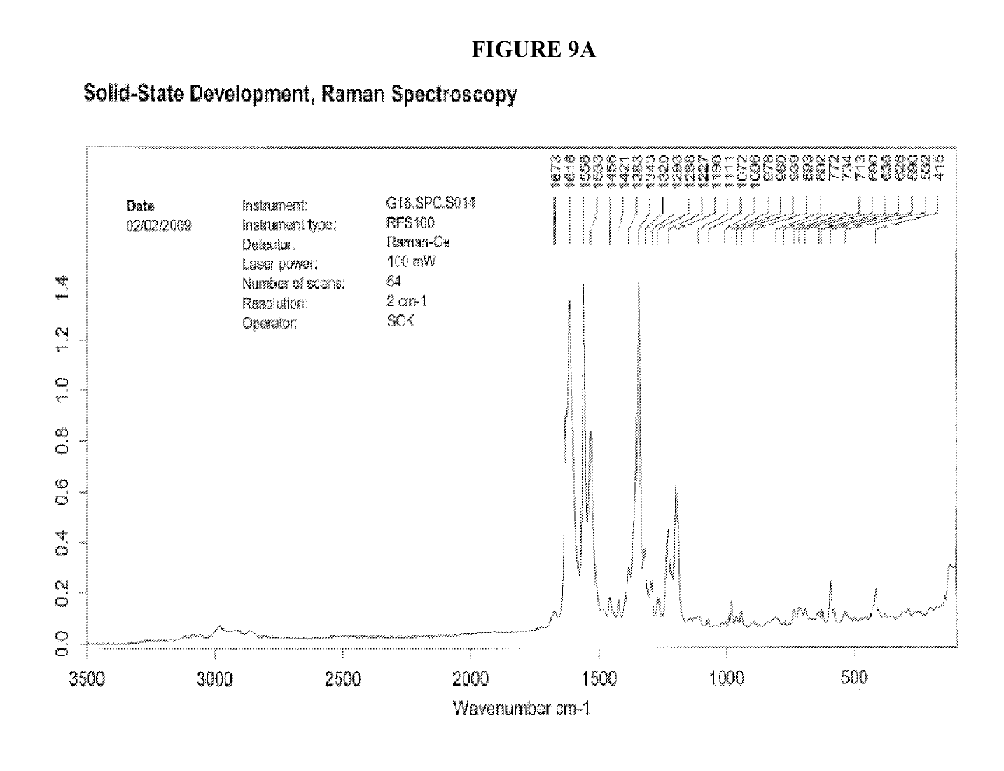

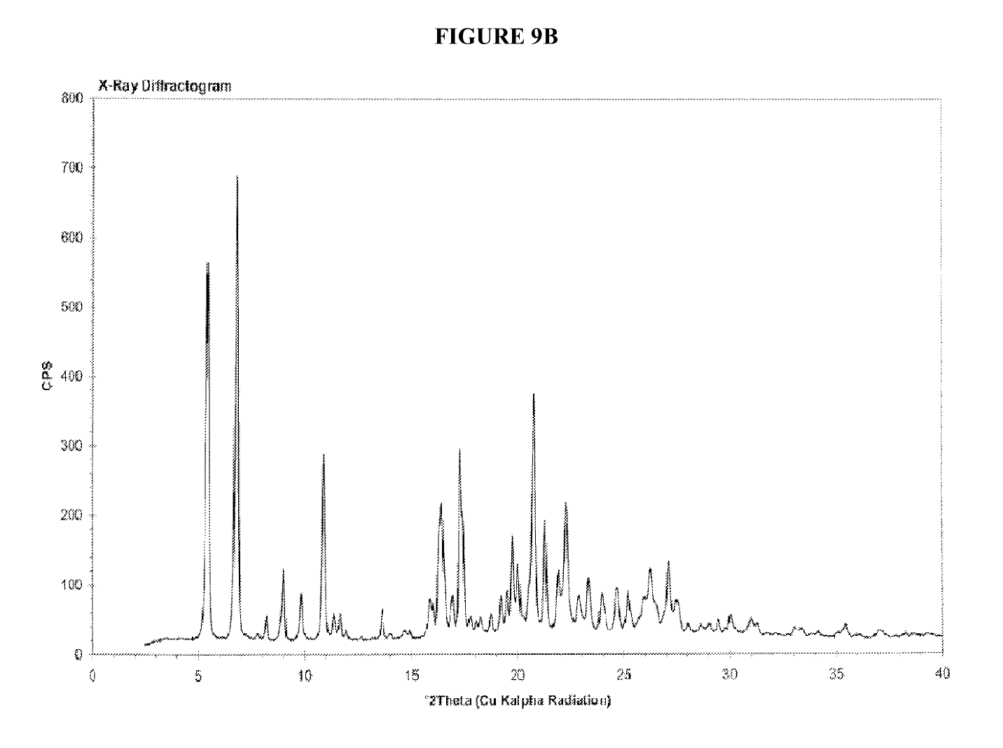

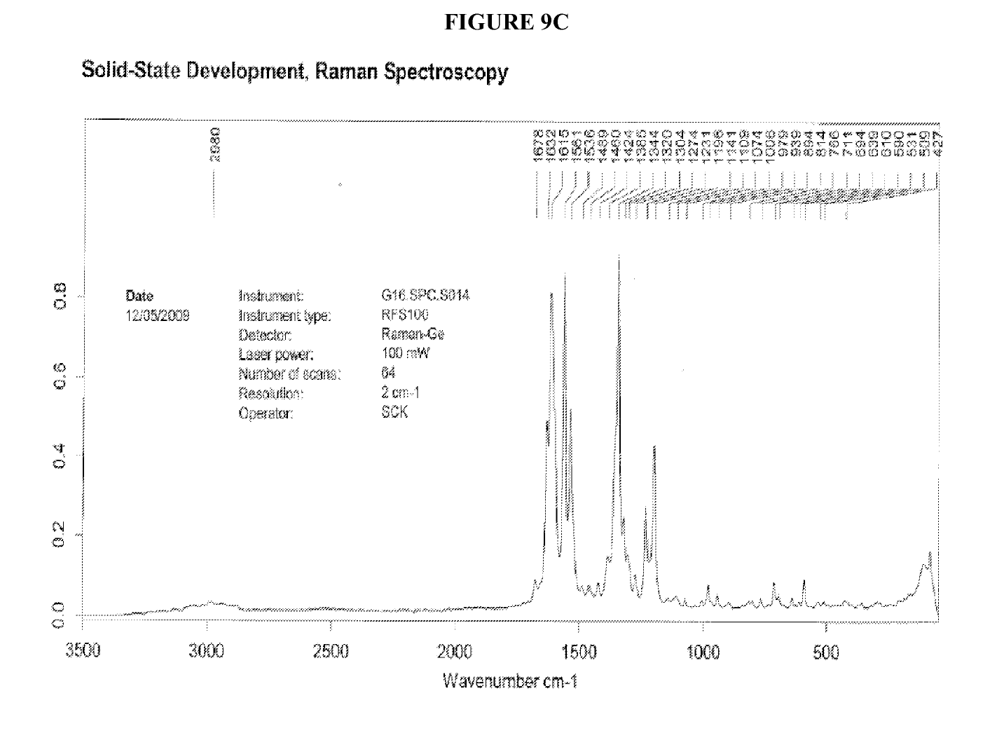

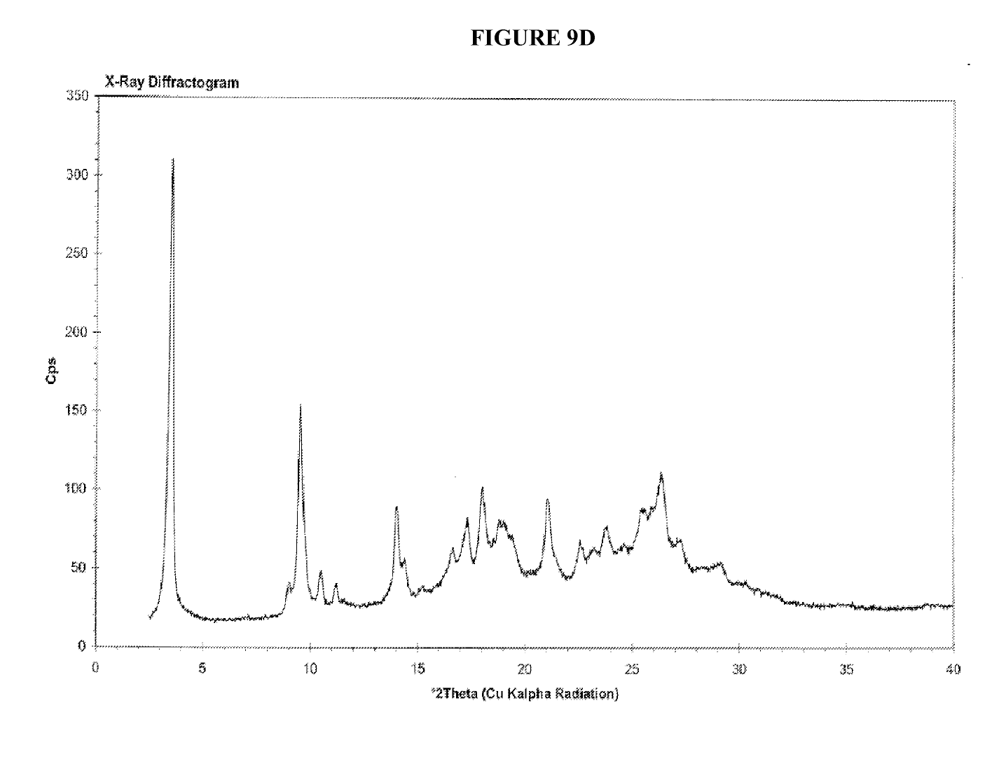

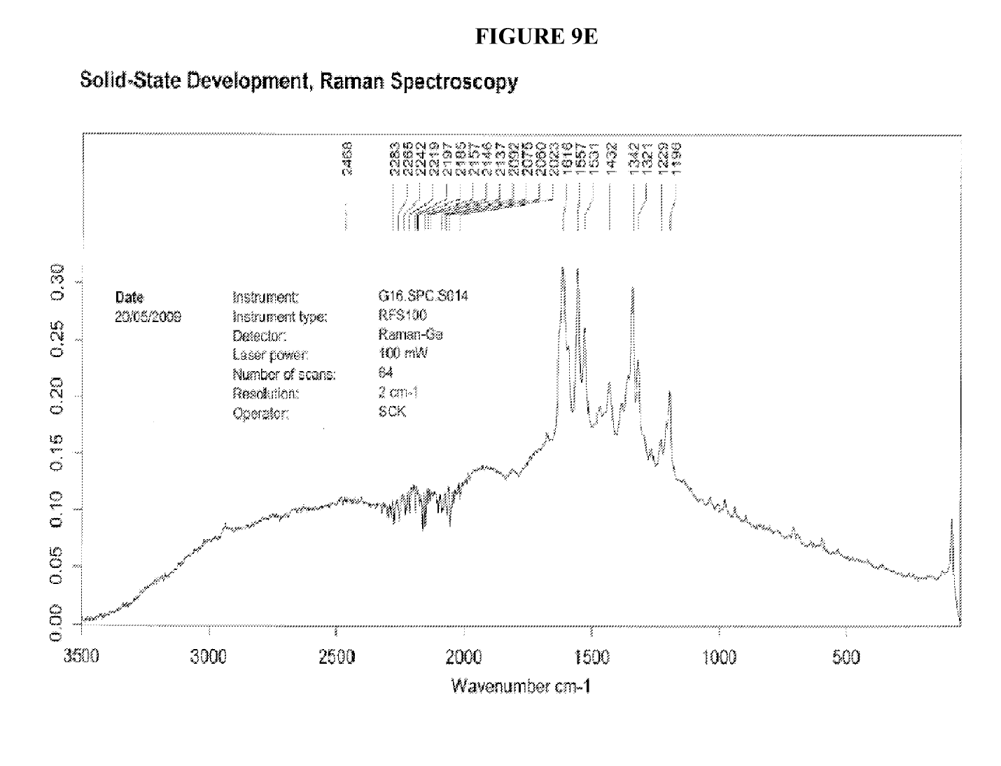

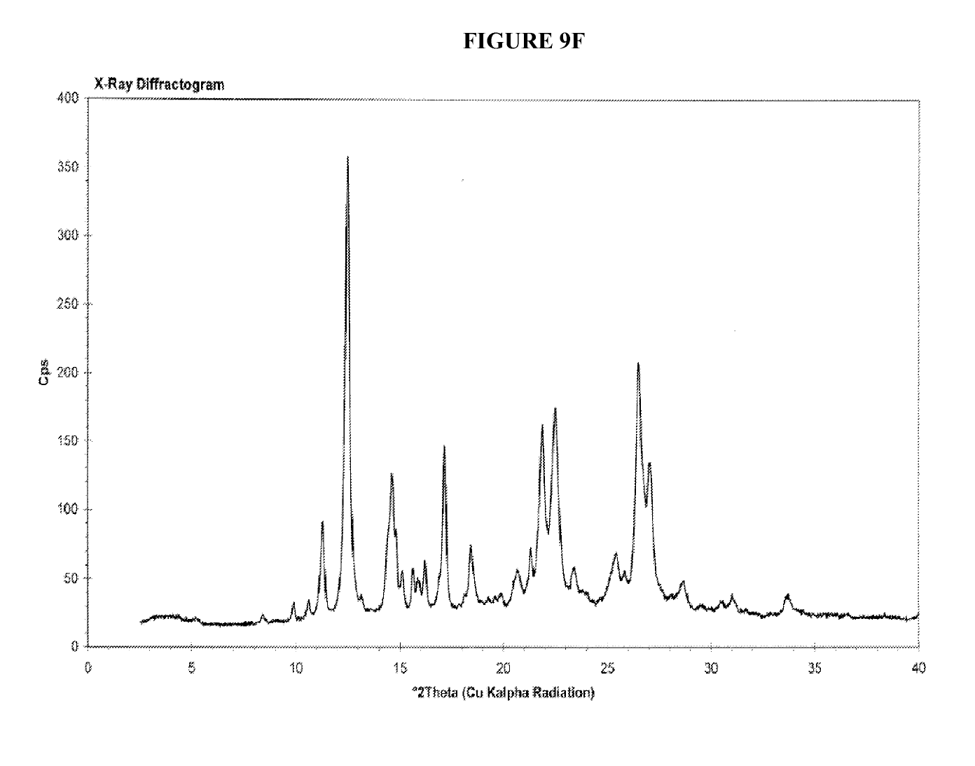

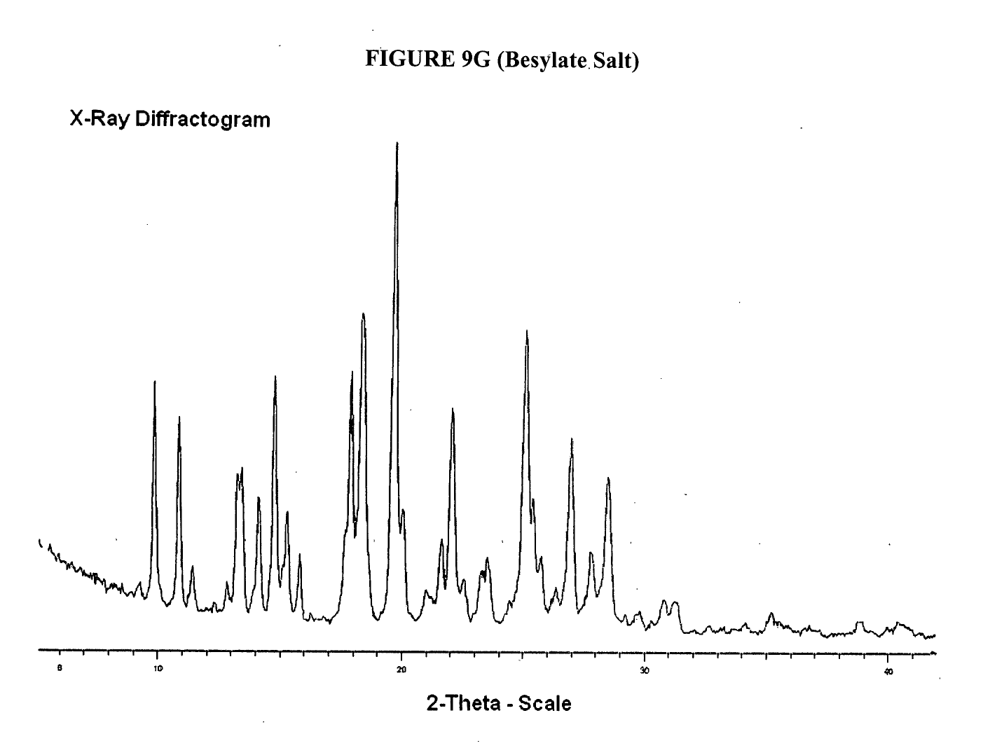

The crystallinity of Compound 1 as a free base and of the corresponding fumarate, mesylate and besylate salts was determined by Raman spectroscopy and PXRD, as presented in FIGS. 9A, 9B, 9C, 9D, 9E, 9F, 9G, 10A, 11A, 11B, and 11C. All forms are crystalline. 10B shows the amorphous besylate form.

### Example 10

### Salts Stress Testing

The chemical stability of the salts produced is studied under a variety of conditions, and the samples are then analysed by UPLC.

Typically, for one salt, 6 samples are kept at 40° C., 60° C., 80° C., 40° C. at 75% RH, 60° C. at 75% RH, and 80° C. at 75% RH, over 8 days, after which they are analysed by UPLC.

For example, when the besylate salt was stored at 40° C. under 75% RH for 2 weeks, the analysis of the sample revealed a purity of 99.29%, thus indicating no change in chemical purity of the sample.

### Biological Examples

### Example 11

### MAPKAP-K5 Assay

MAPKAP-K5 reactions are performed in FlashPlate format using 0.1 or 0.2 μCi 33P-ATP; 0.6 μM ATP; 1 mU MAPKAP-K5; 3 μM MAPKAP-K5 peptide substrate, incubated at room temperature for 30 minutes.

### Flashplate Assay:

The MAPKAP-K5 kinase reaction is performed in a 384 well polypropylene plate (Matrix Technologies) and then transferred to a streptavidin-coated 384 well flashplate (Perkin-Elmer). To wells containing 2 μL test compound or standard inhibitor, 13 μL Enzyme mix or diluent are added using a Hydra (Robbins Scientific). Reactions are started by addition of 10 μL of [2.5×] substrate cocktail using a Multidrop (Thermo-Labsystems), to give final concentrations in the assay of:

1 mU MAPKAP-K5

3 μM MAPKAP-K5 peptide substrate

0.6 μM ATP

0.004 μCi [33P]-γ-ATP/μL

1× reaction buffer

Plates are incubated at room temperature for 30 minutes. Reactions are terminated by the addition of 25 μL EDTA (50 mM) to each well using a Micro-fill (Biotek). Reactions are transferred to a streptavidin-coated flashplate using a Zymark robotic system. Plates are incubated for 60 minutes at room temperature. All wells are washed 3 times with 100 μL phosphate buffered saline using a Tecan plate washer. Radioactivity is determined by scintillation counting of the flashplate (empty wells) on a Packard TopCount.

Enzyme Mix:
Enzyme
50 mM Tris Hcl (pH 7.5)
0.1 mM EGTA
2 mM DTT
1 mg/mL BSA

Reaction Buffer:
50 mM Tris Hcl (pH 7.5)
0.1 mM EGTA
10 mM Magnesium acetate
2 mM DTT

Compounds 1 and 2 have been or can be prepared according to the synthetic methods described above. The activity of each compound, which can be determined using the MAPKAPK5 assay method described in Example 12, is between 0.1 and 100 nM:

### Example 12

### Development of an Assay for the Identification of Regulators of the Expression of MMP1 by Activated Primary Synovial Fibroblasts

To identify compounds that decrease the ECM-degrading activity of cells, the ECM-degrading activity of cells may be induced to allow proper detection of this activity, and to achieve a clearer read-out. In the context of RA, the cells of choice are mammalian synovial fibroblasts and the triggers that may be used to induce the ECM-degrading activity are cytokines relevant in the field of arthritis: for instance TNF-α, IL1β, IL6, OSM, IL17, and MIF1-α. This list is not comprehensive due to the plethora of cytokines potentially involved in the RA pathogenesis (Smolen and Steiner, 2003). To set up an in vitro assay that is as close as possible to the complexity of the pathology, the trigger applied should be a mixture of factors generated by contacting cytokine-producing cells relevant in the field of arthritis, such as monocytes, macrophages, T-cells, and B-cells, with a trigger. The cytokine-producing cells will respond to the contact by producing a complex and unbiased mixture of factors. If the cytokine-producing cell used is also found in a pannus, and the cytokine applied to produce this trigger is found in the synovial fluid of rheumatoid arthritis patients, the mixture of factors ultimately produced will contain part of the factors that are present in the joints of arthritis patients.

### Principle of the ‘MMP Assay’

Matrix Metallo Proteases (MMPs) possess various physiological roles, as e.g. the maturation of other proteases, growth factors, and the degradation of extra-cellular matrix components. MMP1 is one of the members of the MMP family that is able to degrade native collagen, the main component of bone and cartilage. An increased expression of MMP1 by synovial fibroblasts (SFs) is diagnostic for the progression of the arthritic disease and is predictive for erosive processes in the joint (Cunnane et al., 2001). The expression of MMP1 by SFs can be increased by the activation of SFs with triggers relevant for rheumatoid arthritis, as cytokines like TNF-α or IL1β (Andreakos et al., 2003). Taken together, measurement of the levels of MMP1 produced by activated SFs is a readout that is highly relevant in the context of RA as this event reflects the level of activation of SFs towards an erosive phenotype as it is seen in the pannus. If a reduced expression of a candidate drug target in activated SFs leads to the reduction of MMP1 expression by these cells, the drug target is then proven to be involved in the regulation of MMP1 expression and thus considered relevant for the development of therapeutic strategies for the treatment of RA.

In the following examples, the development of an assay, further referred to as ‘MMP assay’, monitors the MMP1 production by synovial fibroblasts (SFs) in response to diverse activating triggers (Example 9.1). The use of this assay is then described for the validation of gene products that are considered drug targets for the development of RA therapies (Example 9.2). The validation of drug targets is performed using recombinant adenoviruses, further referred to as knock-down viruses or Ad-siRNAs, that mediate the expression in cells of shRNA's which reduce the expression levels of targeted genes by a RNAi (RNA interference)-based mechanism (see WO 03/020931). The use of the ‘MMP assay’ for the testing of compounds that modulate the activity of the drug targets identified is described further below.

### Assay Examples

### Control Viruses Used

The control viruses used in these studies are listed below. dE1/dE2A adenoviruses are generated from these adapter plasmids by co-transfection of the helper plasmid pWEAd5AflII-rITR.dE2A in PER.E2A packaging cells, as described in WO99/64582.

### Negative Control Viruses:

Ad5-eGFP_KD: Target sequence: GCTGACCCTGAAGTTCATC (SEQ ID NO: 1). Cloned using Sap1-sites into vector and virus generated as described in WO03/020931.
Ad5-Luc_v13_KD: Target sequence GGTTACCTAAGGGTGTGGC (SEQ ID NO: 2). Cloned using Sap1-sites into vector and virus generated as described in WO03/020931.
Ad5-M6PR_v1_KD: Target sequence CTCTGAGTGCAGTGAAATC (SEQ ID NO: 3). Cloned using Sap1-sites into vector and virus generated as described in WO03/020931.

### Positive Control Viruses:

Ad5-MMP1_v10_KD: Target sequence ACAAGAGCAAGATGTGGAC (SEQ ID NO: 4). Cloned using Sap1-sites into vector and virus generated as described in WO03/020931.

### Viruses Used for Target Validation:

Ad5-MAPKAPK5_v13_KD: Target sequence CGGCACTTTACAGAGAAGC (SEQ ID NO: 5). Cloned using Sap1-sites into vector and virus generated as described in WO03/020931.
Ad5-MAPKAPK5_v12_KD: Target sequence ATGATGTGTGCCACACACC (SEQ ID NO: 6). Cloned using Sap1-sites into vector and virus generated as described in WO03/020931.

### Example 12.1

### Development of the MMP Assay

A 384-well format ELISA for measurement of MMP1 is developed. Various primary antibodies are tested, as well as various ELISA protocols. The following protocol is developed and validated to measure MMP1 levels in SF supernatant in 384 well plates: white Lumitrac 600 384 well plates (Greiner) are coated with 2 μg/mL anti-MMP1 antibody MAB1346 (Chemicon). The antibody is diluted in buffer 40 (1.21 g Tris base (Sigma), 0.58 g NaCl (Calbiochem) and 5 mL 10% NaN₃ (Sigma) in 1 L milliQ water and adjusted to pH 8.5). After overnight incubation at 4° C., plates are washed with PBS (80 g NaCl, 2g KCl (Sigma), 11.5 g Na₂HPO₄.7H₂O and 2 g KH₂PO₄ in 10 L milliQ; pH 7.4) and blocked with 100 μL/well Casein buffer (2% Casein (VWR International) in PBS). Next day, casein buffer is removed from ELISA plates and replaced by 50 μL/well EC buffer (4 g casein, 2.13 g Na₂HPO₄ (Sigma), 2 g bovine albumin (Sigma), 0.69 g NaH₂PO₄.H₂O (Sigma), 0.5 g CHAPS (Roche), 23.3 g NaCl, 4 mL 0.5 M EDTA pH 8 (Invitrogen), 5 mL 10% NaN₃ in 1 L milliQ and adjusted to pH 7.0). 0.25 mM DTT (Sigma) is added to the thawed samples plates. After removal of the EC buffer, 20 μL of sample is transferred to the ELISA plates. After overnight incubation at 4° C. plates are washed twice with PBS and once with PBST (PBS with 0.05% Tween-20 (Sigma)) and incubated with 35 μL/well biotinylated anti-MMP1 antibody solution (R&D). This secondary antibody is diluted in buffer C (0.82 g NaH₂PO₄.H₂O, 4.82 g Na₂HPO₄, 46.6 g NaCl, 20 g bovine albumin and 4 mL 0.5M EDTA pH 8 in 2 L milliQ and adjusted to pH 7.0) at a concentration of 5 μg/mL. After 2 h of incubation at RT, plates are washed as described above and incubated with 50 μL/well streptavidin-HRP conjugate (Biosource). Streptavidin-HRP conjugate is diluted in buffer C at a concentration of 0.25 μg/mL. After 45 min, plates are washed as described above and incubated for 5 min with 50 μL/well BM Chem ELISA Substrate (Roche). Readout is performed on the Luminoscan Ascent Luminometer (Labsystems) with an integration time of 200 msec or with an Envision reader (Perkin Elmer).

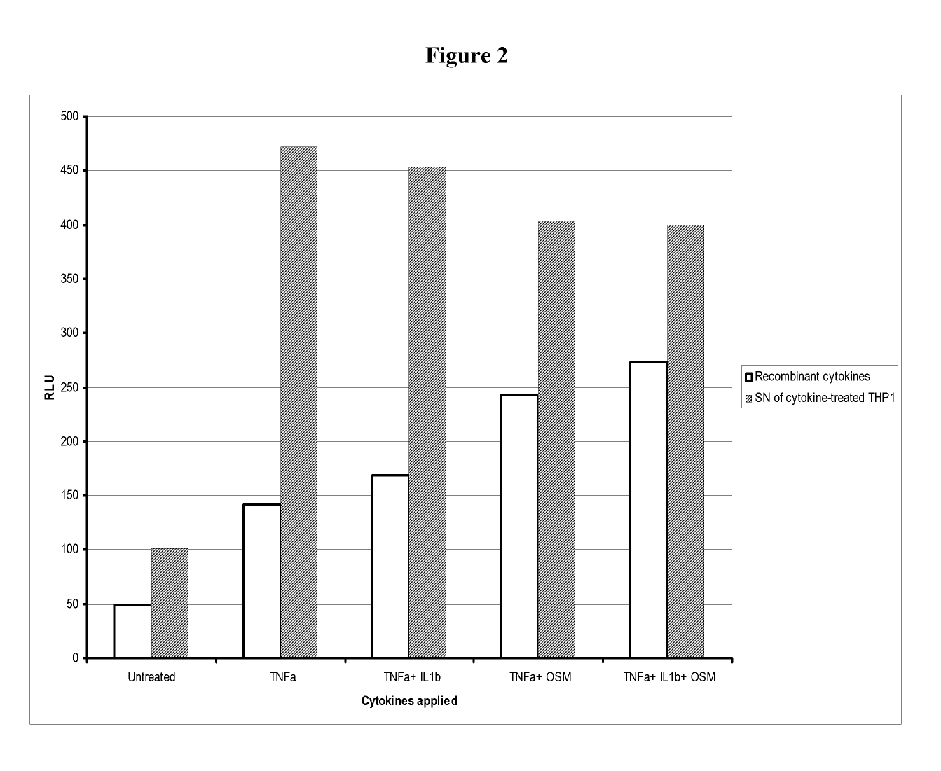

The increase of MMP1 expression by SFs upon treatment with cytokines relevant in the field of RA (TNF-α, IL1β and OSM) or a combination thereof is shown in FIG. 2 as white bars. For this experiment, SFs are seeded in 96 well plates, 3,000 cells/well. 24 h later, the medium is changed to M199 medium supplemented with 1% FBS. One day after the medium change, cytokines or combinations thereof are added to the cultures, each cytokine being added to a final concentration of 25 ng/mL. 72 h after cytokine addition, the supernatant is collected and processed in the MMP1 ELISA as described in the protocol given above. In parallel with this experiment, SFs are triggered, using the same protocol, with the supernatant of THP1 cells (2-fold diluted in M199+1% FBS) treated with the same cytokines or combinations of cytokines for 48 h in M199 medium+1% FBS. MMP1 levels for these samples are shown in FIG. 2 as grey bars. The induction of the MMP1 expression by SFs triggered with the supernatants of TNF-α-treated THP1 cells is stronger (>4.5 fold induction) as compared to the SFs triggered with recombinant TNF-α alone (3-fold induction) and almost equals the 5-fold induction obtained by a mixture of 3 purified cytokines (TNF-α, IL1βb, OSM). This result indicates that the supernatant of TNF-α-induced THP1 cells contains, besides TNF-α, additional pro-inflammatory factors that activate SFs towards MMP1 expression. As the role of TNF-α in the RA pathogenesis is validated (TNF-α-blockers such as Infliximab and Etanercept show some efficacy in the treatment of RA patients) and the THP-1 cells are representative for monocytes/macrophages present in the joint of RA patients, the TNF-α-based trigger mixture prepared by contacting THP-1 cells with TNF-α will contain factors present in the joints of RA patients and subsequently is relevant to RA. This TNF-α-based complex trigger, further referred to as the ‘complex trigger’, will further be used as basis for the ‘MMP assay’.

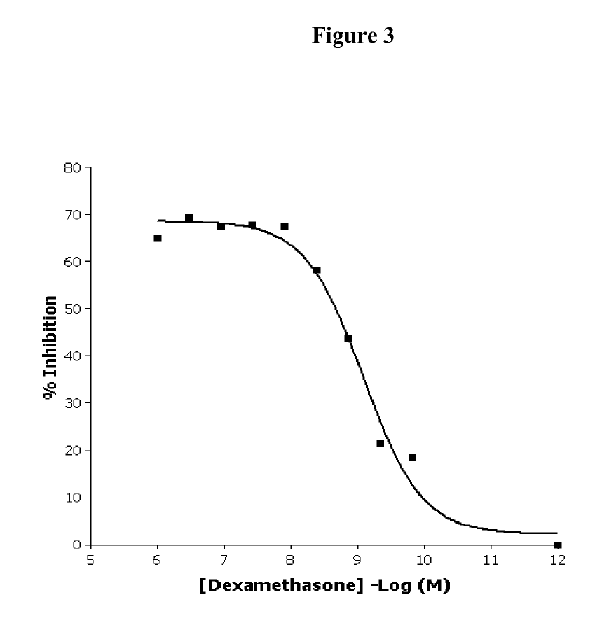

Inhibition of the activation of SF by the ‘complex trigger’ is shown using dexamethasone, a potent anti-inflammatory agent that also strongly reduces collagen-induced arthritis in rodents (Yang et al., 2004) (FIG. 3). Dexamethasone is shown to dose-dependently reduce amounts of MMP1 produced by complex trigger activated SFs. SFs are seeded at a density of 3000 cells/well in 96 well plates. 24 hrs after seeding, increasing concentrations of dexamethasone are added to the cells. After overnight incubation, medium of every well is refreshed to supernatant of THP-1 cells treated with TNF-α (50% diluted in M199+0.5% FBS), and the same concentration of dexamethasone as added the day before. 48 hrs after treatment, the supernatant is collected and subjected to the MMP1 ELISA described above. The addition of dexamethasone clearly reduced the MMP1 expression by SFs, with an IC₅₀ value of about 1 nM (see FIG. 3). These data show that the MMP1 expression by activated SFs can be reduced by the addition of a physiologically relevant inhibitor and represent a proof of principle for the ‘MMP assay’.

### Example 12.2

### MAPKAPK5 Modulates SF ‘Complex Trigger’-Induced MMP1 Expression

### (A) Ad-siRNA Virus Functions to Knock Down MAPKAPK5 Expression.

Recombinant adenoviruses mediating the expression of siRNA's targeting MAPKAPK5 and eGFP are generated according to the procedure described in WO03/020931. The target sequence used in the recombinant adenovirus is: CGGCACTTTACAGAGAAGC (SEQ ID NO: 5) as well as ATGATGTGTGCCACACACC (SEQ ID NO: 6). The target sequence within the eGFP mRNA used in the recombinant adenovirus is: GCTGACCCTGAAGTTCATC (SEQ ID NO: 1). These sequences are cloned into the adapter plasmid using Sap1 sites. dE1/dE2A adenoviruses are generated from these adapter plasmids by co-transfection of the helper plasmid pWEAd5AflII-rITR.dE2A in PER.E2A packaging cells, as described in WO99/64582.

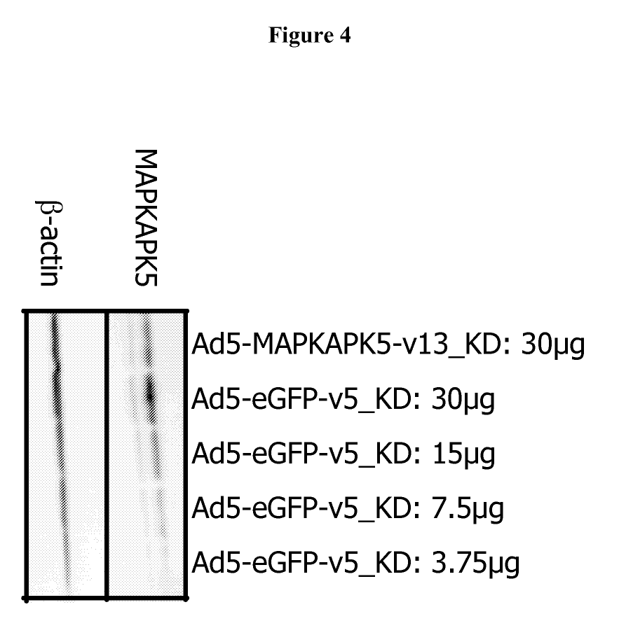

The functionality of an adenovirus targeting MAPKAPK5 is tested as follows. These adenoviruses are used to infect primary human SFs cultured in petri dishes as follows. On day 1, 500.000 SFs are seeded per petri dish. One day later, the cells are infected with Ad5-MAPKAPK5-v13_KD (1.6E9 VP/mL) or Ad5-eGFP-v5_KD (1.3E10 VP/mL) at an MOI of 4000 (based on the titers (number of virus particles per mL) defined for the viruses by Q-rt-PCR). On day 7, cells are detached from the petri dish according to standard procedure using a trypsin EDTA solution. The trypsin is then neutralized by addition of DMEM growth medium supplemented with 10% FBS. The cells are then collected by a centrifugation step (1000 rpm, 5 min). The pellet is lysed in 100 μL of fresh RIPA buffer (50 mM Tris pH7.5, 150 mM NaCl, 1% deoxycholate, 1% Triton X100, 0.1% SDS). The samples are then sonicated for 10 sec. The protein concentration of the samples is then determined using the BCA kit (Pierce, Cat N^{o} 23227) as described by the provider, using BSA as a standard. To 30 μg of cell lysate diluted to 19.5 μl in RIPA buffer, 3.5 μL of reducing agent (NuPage reducing agent N^{o} 10, Invitrogen NP0004) and 7.5 μL of sample buffer (NuPage LDS sample buffer, Invitrogen NP0007) are added. The 30 μL sample is then boiled for 5 min and loaded on a 10% polyacrylamide gel (Invitrogen NP0301). To allow the estimation of the level of protein knock-down, 15 μg, 7.5 μg and 3.75 μg of the lysate of the Ad5-eGFP-v5_KD infected cells are also loaded onto the gel. The gel is then run for 2 hours at 100V in 1×MOPS/SDS NuPage running buffer (Invitrogen NP001). 10 μl of Seablue Plus Prestained standard (Invitrogen LC5925) is used to estimate protein size on the gel. The proteins on the gel are then transferred onto a PVDF membrane (Invitrogen LC2002) by a wet blotting procedure using a transfer buffer prepared by mixing 100 mL Nupage Transfer buffer 20* (NP0006-1), 400 mL methanol and 1500 mL Milli Q water. Before the transfer, the membrane is first soaked in methanol and in transfer buffer. The transfer is performed at 100V for 90 minutes. The membrane is then blocked by 30 min soaking in blocking buffer (2% blocking blocking powder (Amersham, RPN 2109) prepared in PBST (PBS supplemented with 0.1% Tween 20 (Sigma, P1379)). After blocking, the immunodetection is performed using a mouse monoclonal antibody against MAPKAPK5 (BD Biosciences, Cat N^{o} 612080) diluted 250 fold in blocking buffer. After overnight incubation with this primary antibody, the membrane is washed 3 times with PBST and incubated 1 hr with the secondary antibody ((Polyclonal goat anti-mouse Ig, HRP conjugated (DAKO P0447) diluted 50000 fold in blocking buffer. The blot is then washed 3 times in PBST and the detection is performed with ECL advance (RPN2109, Amersham) on a Kodakimager according to the manufacturers instructions. The Western Blotting revealed a lower expression level of MAPKAPK5 in the Ad5-MAPKAPK5-v13_KD infected cells compared to the cells infected with the Ad5-eGFP-v5_KD negative control virus. Comparison with the diluted Ad5-eGFP-v5_KD infected samples allowed to estimate the reduction in expression to be 2-fold. Equal loading of the 30 μg samples is demonstrated by immunodetection of β-actin after removal of the MAPKAPK5 antibody by a ‘stripping procedure’ (5 minutes boiling of the membrane in PBST) Immunodetection of β-actin is performed according to the method described for MAPKAPK5 detection, but using a goat polyclonal antibody against β-actin (Santa Cruz, Cat N^{o} SC-1615) at a 1000 fold dilution as primary antibody and a rabbit anti goat antibody at a 50000 fold dilution as a secondary antibody. Results of this experiment are given in FIG. 4. Taken together, this experiment demonstrated the functionality of the Ad-siRNA virus produced to reduce the MAPKAPK5 expression levels in primary human SFs.

### (B) MAPKAPK5 Knock-Down Ad-siRNA Reduces SF-Induced MMP1 Expression

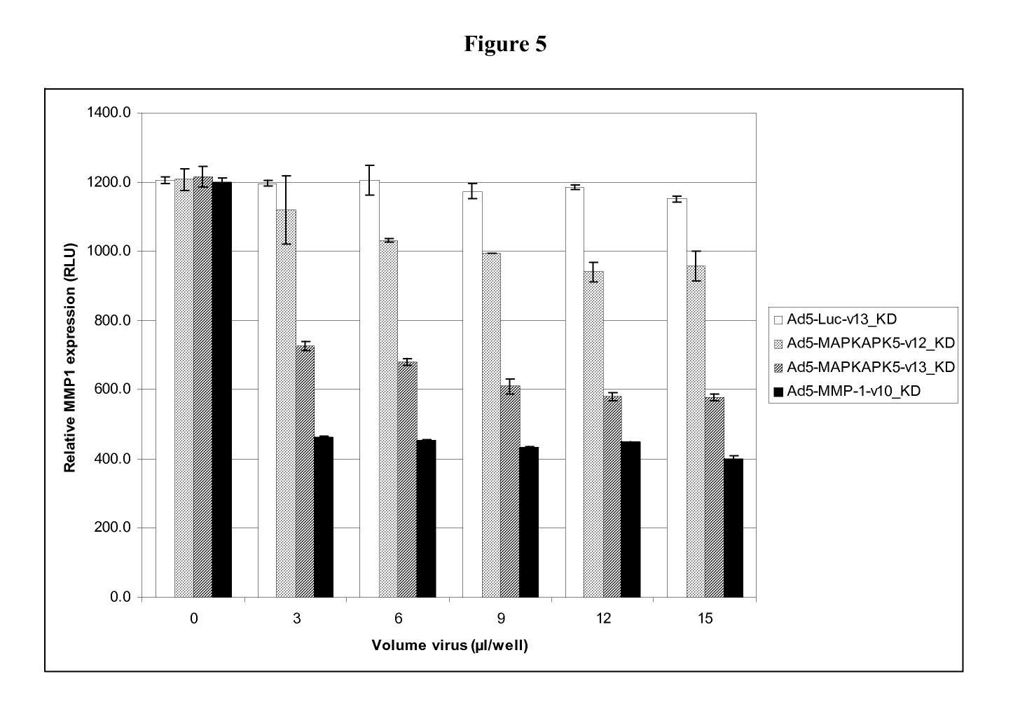

The efficacy of Ad5-MAPKAPK5-v13_KD virus in the ‘MMP assay’ is tested as follows. Day 1, SFs (passage 9 to 10) are seeded in 96 well plates at a density of 3000 cells per well in complete synovial growth medium (Cell Applications). One day later, the cells are infected with increasing amounts (3, 6; 9, 12 or 15 μl) of following viruses: Ad5-eGFP-v5_KD, Ad5-MAPKAPK5-v12_KD, Ad5-MAPKAPK5-v13 KD, Ad5-MMP1-v10_KD. The virus load is corrected by addition of the neutral virus Ad5-Luc-v13_KD to bring the final virus volume on the cells to 15 μL in every well. This correction guarantees that the effects observed do not result from the virus load applied to the cells. The cells are then incubated for 5 days before the activation step. This step involves the replacement, in every well, of the growth medium by 75 μL of M199 medium supplemented with 25 μL of ‘complex trigger’. 48 hrs after the activation step, the supernatant is collected and subjected to the MMP1 ELISA as described in Example 1. The results of the experiment are shown in FIG. 5. The quality of the experiment is demonstrated by the efficacy of the Ad-siRNA virus targeting MMP1 itself. This positive control virus strongly reduces the MMP1 expression by SFs, whereas the negative control virus, designed to target the expression of luciferase, does not influence the levels of MMP1 expression. Two viruses used to validate the MAPKAPK5 target (Ad5-MAPKAPK5-v12_KD and Ad5-MAPKAPK5-v13) do also lead to a clear reduction of the complex trigger induced MMP1 expression by primary human SFs. It can be concluded, from this experiment, that MAPKAPK5 represents a valuable drug target that is shown to modulate MMP1 expression in SFs. Similarly, the inhibition of MAPKAPK5 enzymatic activity by a small molecule compound is expected to reduce the ‘complex cytokine’ induced MMP1 expression in the ‘MMP assay’. The inhibition of MAPKAPK5 enzymatic activity by a small molecule compound is also predicted to reduce the degradation of the joint associated with RA.

### (C) In Vitro ‘MMP Assay’ Testing of Compounds Inhibiting MAPKAPK5

Compounds inhibiting the MAPKAPK5 activity in a biochemical assay (i.e. cell free, using purified enzyme), are tested in the ‘MMP assay’ according to following protocol.

The compound master stocks (all at 10 mM concentration in 100% DMSO) are diluted 10-fold in water (Distilled water, GIBCO, DNAse and RNAse free) to obtain a 1 mM intermediate work stock in 10% DMSO. This intermediate work stock is further diluted either 3-fold (or 10-fold) in 10% DMSO to obtain an intermediate work stock of 333 μM (or 100 μM) concentration, respectively, in 10% DMSO. The 1 mM as well as 333 μM (or 100 μM) intermediate work stocks are then further diluted 10-fold in 1.1% DMSO to obtain the 10× workstocks at 100 μM and 33.3 μM (or 10 μM) concentration in 2% DMSO. This 10× work stock is then diluted 10-fold in M199 medium supplemented with 1% FBS to obtain the final ‘1× compound preparation’ containing the compounds at 10 μM and 3.33 μM (or 1 μM) as well as 0.2% DMSO. These are the final conditions at which the compounds are tested on the cells. In parallel, the 10× work stock is diluted 10-fold in ‘complex trigger’ (i.e. the supernatant of TNF-α treated THP1 cells produced as described in Example 1) that is diluted 2-fold in M199 supplemented with 1% FBS to produce the ‘1× compound in 50% complex trigger preparation’.

At day 1, RASFs are seeded in 96 well plates (Flat bottom, tissue culture treated, Greiner) at a density of 3000 cells/well in complete synovial growth medium (Cell Applications). Day 5, the compounds are added to the cultured cells as follows. Medium is completely removed from the cells and replaced by 75 μL of the ‘1× compound preparations’ containing the compounds at either 10 μM or 3.33 μM (or 1 μM) in M199 medium supplemented with 1% FBS and 0.2% DMSO. After an incubation period of 2 hours, which allows the compounds to equilibrate and enter the cells, 25 μL of the ‘1× compound in 50% complex trigger preparations’ are added to the wells on top of the ‘1× compound preparation’, in the wells containing the corresponding compounds at corresponding concentration. In this way, an 8-fold diluted complex trigger is ultimately applied to the cells. An incubation of 48 hrs is then performed and 20 μl of the cell supernatant is then processed in the MMP1 ELISA as described above, delivering raw data (RLU: relative luminescence units). Following controls are included in the experiments. A maximal signal control, in which the cells are activated by the complex trigger but only the 0.2% DMSO vehicle (and thus, no compound) is added. This control indicates the maximal level of MMP1 that can be achieved in the test. A minimal signal control is also included in these experiments. Here, cells are not triggered. The medium of the cells is then changed to 100 μM199 medium supplemented with 1% FBS at day 5. This control returns the basal MMP1 levels produced by the RASFs. The percent inhibition of the MMP1 expression achieved by the compounds is then calculated based on the RLU data returned by the ELISA with following formula:

[[(maximal MMP1 levels−minimal MMP1 levels)−(MMP1 level compound X at concentration Y−minimal MMP1 levels)]/(maximal MMP1 levels−minimal MMP1 levels)]×100.

Toxicity of the compounds is assessed as follows. Day 1, SFs are seeded in white, tissue culture treated 96 well plates at a density of 3000 cells per well in 100 μL complete synovial growth medium. The compound handling, compound addition to the cells as well as activation of the cells is further performed as described above in this example for the determination of the MMP1 levels. After the 48 hrs incubation period, the medium is removed from the wells, replaced by 50 μL fresh M199 medium supplemented with 1% FBS. 50 μL of substrate (Promega Celltiter Glow cell viability kit) is then added to the wells. After an incubation period of 10 min, luminescence signal is measured. A reduction of the luminescence signal by more than 50% as compared to the maximal control wells is considered to reflect significant toxicity. No toxicity is observed for the compounds tested in the ‘MMP assay’.

It should be understood that factors such as the differential cell penetration capacity of the various compounds can contribute to discrepancies between the activity of the compounds in the in vitro biochemical and cellular MMP assays.

Using the protocol described above for the determination of MMP1 activity, Compound 1 has an IC₅₀ between 100 and 1000 nM

### Example 13

### Assay to Assess Effect of Compounds on Cytokine Release by Human PBMCs

Human peripheral blood mononuclear cells (PBMCs) are isolated from “buffy coats” prepared from the blood of healthy volunteers, isolated essentially according to method of Bøyum (1984). In brief, buffy coat is diluted 1:1 with 1×PBS (Gibco) and 30 mL is carefully put on top of 20 mL Lymphoprep™ (Lucron Bioproducts) in 50 mL Falcon tubes. After centrifugation (35 min, 400 g, 18° C.) the mononuclear cells are collected from the white interphase and washed 3 times with 1×PBS by resuspending and centrifugation (10 min, 200 g). Isolated PBMCs are finally resuspended in RPMI 1640 (Cat. No. 21875, Gibco) that is supplemented with 10% heat-inactivated FBS (Hyclone).

For the assay PBMCs are seeded at 2.5E6 cells/mL in 160 μL in 96-well plates (Nunc). Serial dilution of the test compounds are made first in DMSO (Sigma) and then diluted 50-fold in M199 medium (Gibco) containing 1% heat-inactivated FBS. Compounds are further 1/10 diluted in the assay plates to obtain final DMSO concentration of 0.2%. Cells are preincubated with the compounds for 1 hr at 37° C., 5% CO₂. Then, cells are stimulated with LPS (Escherichia coli serotype 026:B6, Cat. No. L2654, Sigma) that is added in a volume of 20 μL to a final concentration of 1 μg/mL and cells are further cultured for 24 hr. The plates are centrifuged and the supernatant is collected and stored at −80° C. until analysis of appropriate dilutions in ELISAs.

The following 384-well chemiluminescent ELISA protocol was developed to measure TNFα levels in the supernatant: White Lumitrac 600 384-well plates (Greiner) are coated with (40 μL/well) anti-TNFα capture antibody (Cat. No. 551220, BD Pharmingen) that is diluted to 1 μg/mL in 1×PBS (Gibco). After overnight incubation at 4° C., plates are washed with 1×PBS (80 g NaCl, 2g KCl (Sigma), 11.5 g Na₂HPO₄.7H₂O and 2 g KH₂PO₄ in 10 L milliQ; pH 7.4) and blocked with 100 μL/well buffer B (1×PBS containing 1% BSA (Sigma), 5% sucrose (Sigma) and 0.05% NaN₃ (Sigma)). After 4 hr incubation at RT, blocking buffer is removed and plates are washed once with PBST (1×PBS with 0.05% Tween-20 (Sigma)). Then, 40 μL of sample is transferred to the ELISA plates and plates are incubated at 4° C. Next day, plates are washed 3 times (twice with PBST and once with PBS) and 35 μL/well biotinylated anti-TNFα antibody (Cat. No. 554511, BD Pharmingen) diluted first to a concentration of 250 ng/mL in buffer D (1×PBS with 1% BSA) is added. After 2 h of incubation at RT, plates are washed as described above and 35 μL/well of a 1/2000 dilution of streptavidin-HRP conjugate (Cat. No. SNN2004, Biosource) in buffer D is added. After 45 min, plates are washed as described above and incubated for 5 min with 50 μL/well BM Chemiluminescence ELISA Substrate POD (Roche). Readout is performed on the Luminoscan Ascent Luminometer (Labsystems) with an integration time of 100 msec delivering raw data (RLU: relative luminescence units). The following controls are included in the experiments, a maximal signal control, in which the cells are activated by LPS but only the 0.2% DMSO vehicle (and thus no compound) is added. This control indicates the maximal level of TNFα that can be achieved in the test. A minimal signal control is also included in these experiments. Here, cells are not triggered. This control returns the basal TNFα levels produced by the PBMCs. The percent inhibition (PIN) of the TNFα release, achieved by the compounds is then calculated based on the RLU data returned by the ELISA with following formula: 100−[((TNFα level compound X at concentration Y−minimal TNFα levels)/(maximal TNFα levels−minimal TNFα levels))×100]. Where compounds are tested at 8 concentrations (1/3 serial dilution), EC₅₀-values can be calculated by curve fitting of the means of the PIN data achieved for a compound at each test concentration.

To assay the effect of compounds on the release of IL1 and IL6 by LPS stimulated PBMC cultures, appropriate dilutions of the supernatant can be measured using the same ELISA protocol as described above. Matched pair antibodies for IL1 and IL6 ELISA (all from R&D Systems) may be used as follows: anti-IL1 capture antibody (Cat. No. MAB601) used at 0.5 μg/mL , biotinylated anti-IL1 detection antibody (Cat. No. BAF201) used at 50 ng/mL; anti-IL6 capture antibody (Cat. No. MAB206) used at 1 μg/mL; biotinylated anti-IL6 detection antibody (Cat. No. BAF206) used at 50 ng/mL.

### Example 14

### MMP13 Assay

The protocol of the MMP13 ELISA is described in section 11.1, then testing of compounds on release of IL-1/OSM-driven MMP13 expression in SW1353 chondrosarcoma cell line is described in section 11.2.

### 14.1 MMP13 ELISA Protocol

A 384 well format ELISA for measurement of MMP13 was developed. Various primary antibodies are tested, as well as various ELISA protocols. The following protocol is developed and validated to measure MMP13 levels in supernatant of cell cultures in 384 well plates.

Black maxisorb 384 well plates (Nunc 460518) are coated with 35 μL of a buffered solution containing 1.5 μg/mL anti-MMP13 antibody MAB511 (R&D systems). The antibody is diluted in carbonate-bicarbonate coating buffer (1.59 g Na₂CO₃ (Sigma S-7795) and 2.93 g NaHCO₃ (Sigma S-5761) in 1 L MilliQ water, adjusted to pH 9.6). After overnight incubation at 4° C., wells are washed twice with 100 μL PBST (80 g NaCl, 2g KCl (Sigma), 11.5 g Na₂HPO₄.7H₂O and 2 g KH₂PO₄ in 10 L milliQ water; pH 7.4+0.05% Tween-20 (Sigma)) and blocked with 100 μL/well blocking buffer (5% non fat dry milk in PBS). After overnight incubation at 4° C., wells are washed twice with 100 μL PBST. The PBST is removed and 35 μL of sample is transferred to the ELISA plates. After 4 hr incubation at RT, plates are washed twice with PBST and incubated for 1 hr at 37° C. with 35 μL/well 1.5 mM APMA solution (a 10 mM APMA stock solution is prepared one day before (35.18 mg APMA (Sigma A-9563) in 10 mL 0.1M NaOH (Merck 1.06469.1000) and stored at 4° C. Before use, the 10 mM APMA stock solution is diluted to 1.5 mM in 1×APMA buffer (10×APMA buffer: 500 mM Tris (Roche 708976), 50 mM CaCl₂ (Sigma C-5080), 500 μM ZnCl₂ (Sigma Z-0173), 1.5 M NaCl (Calbiochem 567441), 0.5% Brij35 (Sigma 430 AG-6) and adjust to pH 7.0). After activation of MMP13 by APMA, plates are washed again two times with 100 μL PBST/well and 35 μL of substrate solution is added to each well. Substrate solution is prepared as follows: OmniMMP Fluorescent substrate (Biomol P-126) stock solution (2 mM in DMSO, stored at −20° C.) is diluted in 1× OmniMMP buffer (10× OmniMMP buffer: 500 mM Hepes (Sigma H4034), 100 mM CaCl₂ (Sigma C5080), 0.5% Brij35 (Sigma 430 AG-6; adjusted to pH 7.0) to a final concentration of 0.01 mM. After an overnight incubation at 37° C., the active MMP13 in the sample has cleaved the substrate and released fluorescence. Readout is performed on the EnVision (Perkin Elmer) using 320 nm excitation/405 nm emission filters.

### 14.2 Assessing Effect of Compounds on Cytokine Driven MMP13 Expression in SW1353 Cells

Human chondrosarcoma cell line SW1353 is acquired from ATCC and grown in DMEM supplemented with 10% heat-inactivated FBS and 1× penicillin/streptomycin (Invitrogen) in a humidified 5% CO₂ incubator at 37° C. Aliquots of the cells are frozen and cryopreserved in liquid nitrogen. Starting from a cryopreserved aliquot, cells are further grown by sub-culturing at a 1/5-1/8 ratio twice a week by trypsinisation.

Starting from the compound master stocks (all at 10 mM concentration in 100% DMSO) a 3-fold serial dilution is made in 96-well plates in 100% DMSO. Then, plates are further diluted 50-fold in M199 medium supplemented with 1% heat-inactivated FBS to obtain an intermediate work stock.

At day 1, SW1353 cells are seeded in 96-well plates (flat bottom, tissue culture treated, Greiner) at a density of 15000 cells/well in 120 μL growth medium. The next day, 15 μL compound out of the intermediate work stock is added. After an incubation period of 60 minutes, which allows the compounds to equilibrate and enter the cells, cells are stimulated with a mixture of IL-1β and OSM, added in a volume of 15 μL to obtain final concentrations of 1 ng/mL IL-1β and 25 ng/mL OSM. For that, stocks of IL-1 (10 μg/mL) and OSM (25 μg/mL) (both PeproTech) are diluted to 10 ng/mL and 250 ng/mL respectively, in M199 medium supplemented with 1% FBS. After incubation for 48 hr, the cell supernatant is harvested and an appropriate dilution is processed in the MMP13 ELISA as described above, delivering raw data (RFU: relative fluorescence units). The following controls are included in the experiments: a maximal signal control, in which the cells are activated by the IL1-β/OSM cytokine mixture but only the 0.2% DMSO vehicle (and thus no compound) is added. This controls indicated the maximal MMP13 levels that can be achieved in the test. A minimal signal control, in which cells only receive the 0.2% DMSO vehicle and no trigger, is also included. This control returns the basal MMP13 levels produced by the SW1353 cells. The percent inhibition of the MMP13 expression achieved by the compounds is then calculated based on the RFU data returned by the ELISA with the following formula: [[(maximal MMP13 levels−minimal MMP13 levels)−(MMP13 level compound X at concentration Y−minimal MMP13 levels)]/(maximal MMP13 levels−minimal MMP13 levels)]×100. Based on a plot of percent inhibition vs Log (molar concentration) and curve fitting, IC₅₀ values of a particular compound can be calculated.

Using the protocol described above for the determination of MMP13 activity, Compound 1 has an IC₅₀ between 50 and 500 nM

### In Vivo Studies

### Example 15

### Tolerability of Compounds

This protocol is designed to assess the tolerability of the compounds of the invention in healthy DBA/1J mice to determine the “therapeutic window” as defined by the dosing range between efficacious (mouse therapeutic Collagen-Induced Arthritis model) and toxic doses.

### 15.1 Animals

DBA/1J nude mice are used (CERJ (France)), the mice are 10-11 weeks old, and have a body weight of approx 20g.

### 15.2 Compound Preparation

Compounds are prepared for a dosing regimen of 100 mg/kg/d, po, free base, in a standard volume of injection of 0.1 mL/10 g of mice (equivalent to 10 mg/1 mL). For solution preparations, compounds are dissolved in 0.5% methyl-cellulose and 1% DMSO supplemented with 1 molar equivalent of methanesulfonic acid to ensure full dissolution, once a week.

### 15.3 Experimental Groups

Groups are randomized based on body weight and treated for up to two weeks. Each group contained 5 mice and received either test compound at 100 mg/kg, a comparison compound at 100 mg/kg/d (Compound A) or vehicle on a daily basis in a dosing volume of 200 μL per mouse.

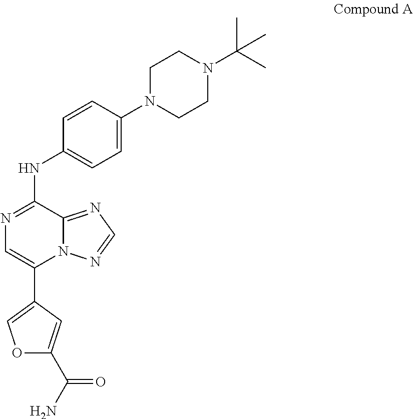

### 15.4 Animal Monitoring (Clinical Signs and Body Weight)

Potential drug toxicity is monitored by observation every day and by recording body weights three times a week for weight loss. All statistical analyses are performed using Student's t test.

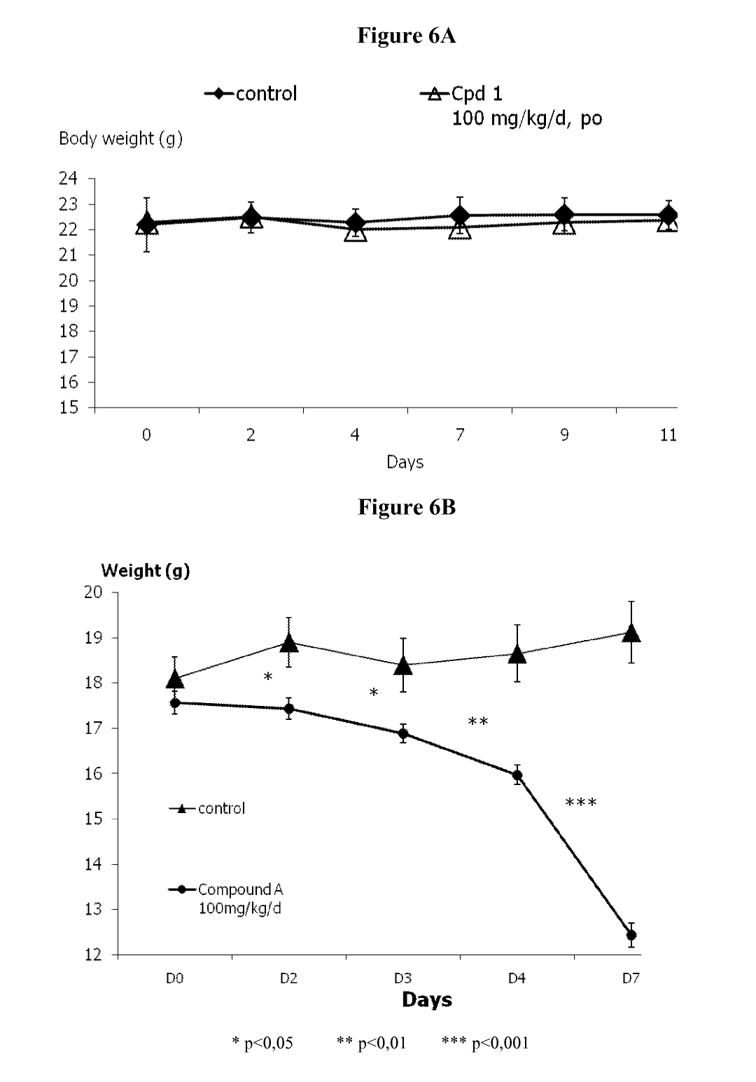

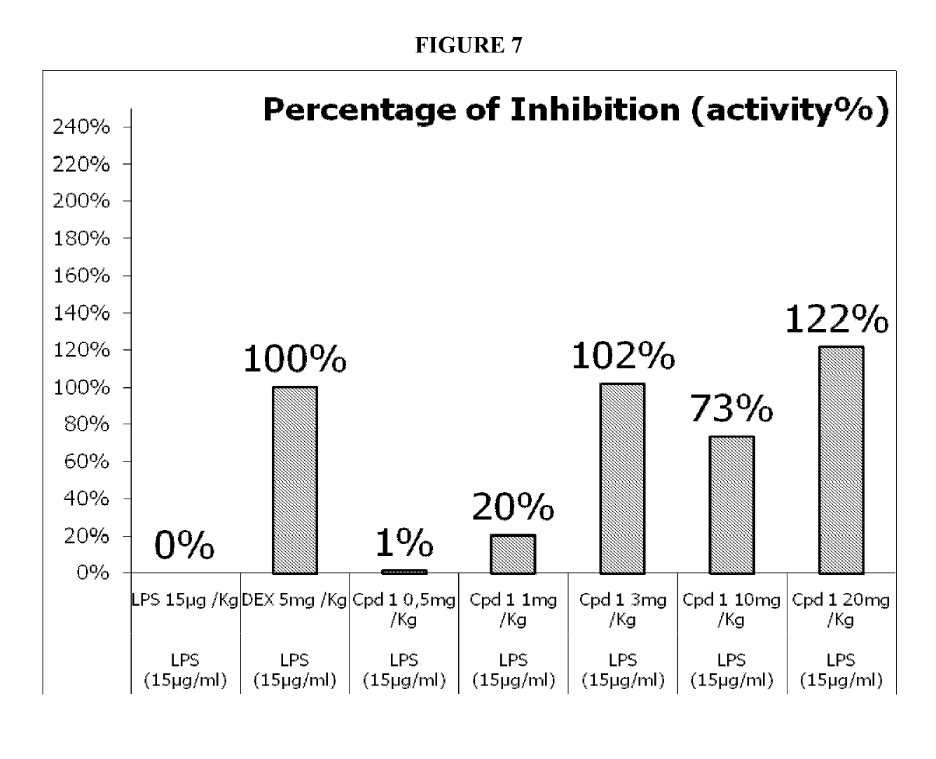

To assess the general tolerability of the treatments, the total body weight is followed throughout the course of the study. FIG. 6A shows the effect of Compound 1 on the total body weight, FIG. 6B shows the effect of a comparator Compound A (see above for structure). The data indicates that the bridged compounds are tolerated better than the non-bridged compounds. In particular there is no statistically significant difference between the weight of the animals treated with Compound 1 at 100 mg/kg/d compared to the vehicle.

### Example 16

### CIA Model

Complete Freund Adjuvant/Collagen II (CFA/Coll II, bovine) is injected (1 mg/mL, 100 μL per animal) into the tail (intradermic) at the start of the experiment. Incomplete Freund Adjuvant /Collagen II (IFA/Coll II) is injected into the tail at the same level (1 mg/mL, 100 μL per animal) 21 days after CFA/Coll II injection.

Animals are then randomized based on score and assigned to treatment groups assuring an equal distribution of score in the different groups.

Treatment with either the test compound (1 mg/kg/d, 3 mg/kg/d or 30 mg/kg/d), positive control (Enbrel, 10 mg/kg/3× week, ip) or vehicle (Methyl Cellulose, 1% DMSO) starts at day 8 post IFA/Coll II-boost (i.e. day 28 of the experiment).

Animals are dosed daily with the test compound, positive control or vehicle for 14 days. The animals are scored daily for clinical symptoms, scoring is reported for the individual paws. During the treatment period the body weight of the animals is monitored. Bone protection is analysed by x-ray imaging.

Compound 1 is efficacious at 3 or 30 mg/kg/d.

### Example 17

### Septic Shock Model

Injection of lipopolysaccharide (LPS) induces a rapid release of soluble tumour necrosis factor (TNF-α) into the periphery. This model is used to analyse prospective blockers of TNF release in vivo.

Six BALB/cJ female mice (20 g) per group are treated at the intended dosing once, po. Thirty minutes later, LPS (15 mg/kg; E. coli serotype 0111:B4) is injected ip. Ninety minutes later, mice are euthanized and blood is collected. Circulating TNF alpha levels are determined using commercially available ELISA kits. Dexamethasone (5 mg/kg) is used as a reference anti-inflammatory compound. Compound 1 is efficacious at 3, 10 and 20 mg/kg, po.

### Example 18

### Mouse Collagen Antibody Induced Arthritis (CAIA) Model (also Called Mouse Monoclonal AntiBody (MAB) Model)

Eight BALB/cJ female mice (20 g) per group are treated at the intended dosing once, po. The same day, 2 mg/mouse of a cocktail of four monoclonal antibodies (MDBiosciences; ref. CIA-MAB-50) is injected i.v. LPS (50 μg/mouse; E. Coli serotype 55:B5) is administered i.p., three days later. Treatment with either the test compound, positive control (Enbrel, 10 mg/kg/3× week, i.p. or dexamethasone, 1 mg/kg, daily, p.o.) or vehicle (Methyl Cellulose, 1% DMSO) starts the day of the antibodies injection Animals are dosed daily with the test compound, positive control or vehicle for up to 10 days. The animals are scored for clinical symptoms and scoring is reported for the individual paws. During the treatment period, the body weight of the animals is monitored.

### REFERENCES

Choy E H, Panayi G S. (2001). N Engl J Med. 344: 907-16.
Firestein G S. (2003). Nature. 423:356-61.
Smolen J S, Steiner G. (2003). Nat Rev Drug Discov. 2: 473-88.
Lee D M, Weinblatt M E (2001). Lancet. 358: 903-11.
Kremer J. M., Westhovens R., Leon M., Di Giorgio E., Alten R., Steinfeld S., Russell A., Dougados M., Emery P., Nuamah I. F., Williams G. R., Becker J.-C., Hagerty D. T., Moreland L. W. (2003) N Engl J Med. 349:1907-1915.
Edwards J. C. W., Szczepanski L., Szechinski J., Filipowicz-Sosnowska A., Emery P., Close D. R., Stevens R. M., Shaw T. (2004) N Engl J Med. 350:2572-2581.
O'Dell J R, Leff R, Paulsen G, Haire C, Mallek J, Eckhoff P J, Fernandez A, Blakely K, Wees S, Stoner J, Hadley S, Felt J, Palmer W, Waytz P, Churchill M, Klassen L, Moore G. (2002) Arthritis Rheum. 46:1164-70.
St Clair E W, van der Heijde D M, Smolen J S, Maini R N, Bathon J M, Emery P, Keystone E, Schiff M, Kalden J R, Wang B, Dewoody K, Weiss R, Baker D; (2004) Combination of infliximab and methotrexate therapy for early rheumatoid arthritis: a randomized, controlled trial. Arthritis Rheum. 50 :3432-43.
Gomez-Reino J J, et al. (2003). Arthritis Rheum. 48: 2122-7.
O'Dell J R. (2004) Therapeutic strategies for rheumatoid arthritis. N Engl J Med. 350(25):2591-602.
New L, Jiang Y, Han J. (2003) Regulation of PRAK subcellular location by p38 MAP kinases. Mol Biol Cell. 14(6):2603-16.
Shi Y, Kotlyarov A, Laabeta K, Gruber A D, Butt E, Marcus K, Meyer H E, Friedrich A, Volk H D, Gaestel M. (2003) Elimination of protein kinase MK5/PRAK activity by targeted homologous recombination. Mol Cell Biol. 23:7732-41.
Seternes O M, Mikalsen T, Johansen B, Michaelsen E, Armstrong C G, Morrice N A, Turgeon B, Meloche S, Moens U, Keyse S M. (2004) Activation of MK5/PRAK by the atypical MAP kinase ERK3 defines a novel signal transduction pathway. EMBO J. 23:4780-91.
Andreakos E, et al. (2003). Arthritis Rheum. 48: 1901-12.
Cunnane G, et al. (2001). Arthritis Rheum 44: 2263-74.
Coussens L M, et al. (2002). Science 295: 2387-92.
Creemers E E, et al. (2001). Circ Res. 2001 89:201-10
Gapski R, et al. (2004). J Periodontol. 75:441-52.
Reif S, Somech R , Brazovski E, Reich R, Belson A, Konikoff F M, Kessler A. (2005) Digestion. 71:124-130.
Rosenberg G A. (2002). Glia. 39:279-91.
Schanstra J P, et al. (2002). J Clin Invest. 110:371-9.
Suzuki R, et al. (2004). Treat Respir Med. 3:17-27.
US 2005/0009832
WO02/056888
WO99/64582
Bundgard, H., Design of Prodrugs, Elsevier, Amsterdam 1985
K. Widder et al, Methods in Enzymology, Ed. Academic Press, 42, (1985)
Advanced Drug Delivery Reviews, H. Bundgard, 8, 1-38, (1992)
H. Bundgaard, et al, J. Pharm. Sci., 77,285 (1988);
N. Nakeya et al, Chem. Pharm. Bull., 32, 692 (1984);
Pro-drugs as Novel Delivery Systems, T. Higuchi and V. Stella, 14 A.C.S. Symposium Series, Bioreversible Carriers in Drug Design, E. B. Roche, ed., American Pharmaceutical Association and Pergamon Press, 1987
Remington's Pharmaceutical Sciences, 17th edition, 1985, Mack Publishing Company, Easton, Pa.
T. W. Greene and P. G. M. Wuts, Protecting Groups in Organic Synthesis, Second Edition, Wiley, New York, 1991
J. H. Jones et al., J. Med. Chem. 1969, 12, 285-87
H. Newman et al., J. Heterocycl. Chem., 1974, 11, 449-451
D. Barlocco et al., J. Med. Chem., 1998, 41, 674-681
DiMauro et al., J. Med. Chem., 2008, 51, 1681-1694
J. M. Keith et al., Bioorg. Med. Chem Lett, 2008; 4838-4843
Lipinsky et al. Adv. Drug Del. Rev., 1997, 23, 3-25

It will be appreciated by those skilled in the art that the foregoing description is exemplary and explanatory in nature, and is intended to illustrate the invention and its preferred embodiments. Through routine experimentation, an artisan will recognise apparent modifications and variations that may be made without departing from the spirit of the invention. Thus, the invention is intended to be defined not by the above description, but by the following claims and their equivalents.

From the foregoing description, various modifications and changes in the compositions and methods of this invention will occur to those skilled in the art. All such modifications coming within the scope of the appended claims are intended to be included therein.

It should be understood that factors such as the differential cell penetration capacity of the various compounds can contribute to discrepancies between the activity of the compounds in the in vitro biochemical and cellular assays.

All publications, including but not limited to patents and patent applications, cited in this specification are herein incorporated by reference as if each individual publication were specifically and individually indicated to be incorporated by reference herein as though fully set forth.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using either ChemDraw® or ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

1. A salt of the compound according to Formula I: 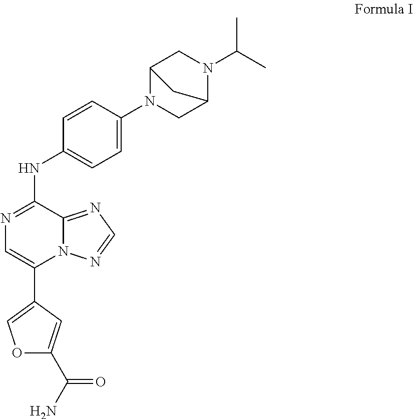
wherein the said salt is a salt formed with adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, or toluenesulfonic acid.

2. The salt according to claim 1, wherein the compound is according to Formula IIa:

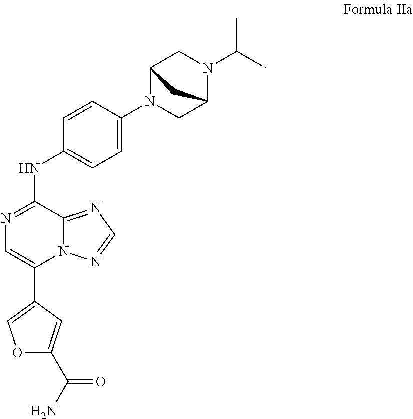

3. The salt according to claim 1, wherein the compound is according to Formula IIb:

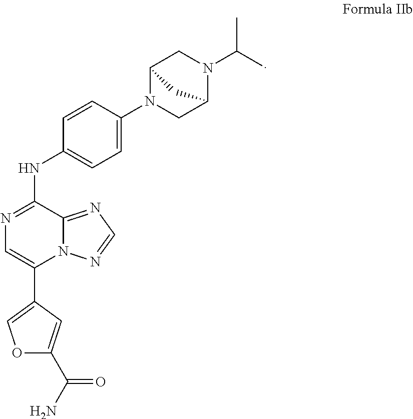

4. The salt according to claim 22, wherein the salt is a salt formed with benzenesulfonic acid, naphthalene-1,5-disulfonic acid or toluene sulfonic acid.

5. The salt according to claim 4, wherein the salt is a salt formed with benzenesulfonic acid.

6. The salt according to claim 22, wherein said salt is in crystalline form.

7. The salt according to claim 22, wherein said salt is a 1:1 free base/salt forming agent adduct.

8. A process for preparing the salt according to claim 22, comprising the steps of
a. reacting the compound of Formula I, IIa or IIb with an acid selected from adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, and toluenesulfonic acid, in an inert solvent; and
b. precipitating the said salt from the said solvent.

9. The process according to claim 8, wherein said compound of Formula I, IIa or IIb and acid are reacted in a molar ratio of between 5:1 and 1:5.

10. The process according to claim 8, wherein said compound of Formula I, IIa or IIb and acid are reacted in a molar ratio of between 2:1 and 1:2.

11. The process according to claim 8, wherein said compound of Formula I, IIa or IIb and acid are reacted in a molar ratio of 1:1.

12. The process according to claim 8, wherein the inert solvent is selected from DMSO, acetone, THF, MTBE, dioxane, EtOAc, MeOH/DCM, and toluene.

13. The process according to claim 8, wherein the inert solvent is selected from iPrOH/water, iPrOH, iBuOH, and tBuOH.

14. A pharmaceutical composition comprising a therapeutically effective amount of a salt according to claim 22 and a pharmaceutically acceptable carrier.

15. A method of treating a mammal susceptible to or afflicted with a condition associated with extra-cellular matrix (ECM) degradation, which method comprises administering an effective amount of a salt according to claim 22, or a pharmaceutical composition according to claim 14.

16. The method of claim 15, wherein said condition is mediated or caused by inflammation.

17. The method of claim 16, wherein said condition is arthritis.

18. The method of claim 16, wherein the condition is rheumatoid arthritis.

19. A method of treating a mammal susceptible to or afflicted with a condition associated with an abnormal cellular expression of MMP1, which comprises administering a therapeutically effective amount of a salt according to claim 22, or a pharmaceutical composition according to claim 14.

20. A method of treatment or prophylaxis of a condition characterized by abnormal matrix metallo proteinase activity, which comprises administering a therapeutically effective matrix metallo proteinase inhibiting amount of a salt according to claim 22, or a pharmaceutical composition according to claim 14.

21. A method of treating a mammal susceptible to or afflicted with diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; and renal disorders, which method comprises administering an effective condition-treating or condition-preventing amount of a salt according to claim 22, or a pharmaceutical composition according to claim 14.

22. A salt of a compound according to Formula I, Formula IIa or Formula IIb: 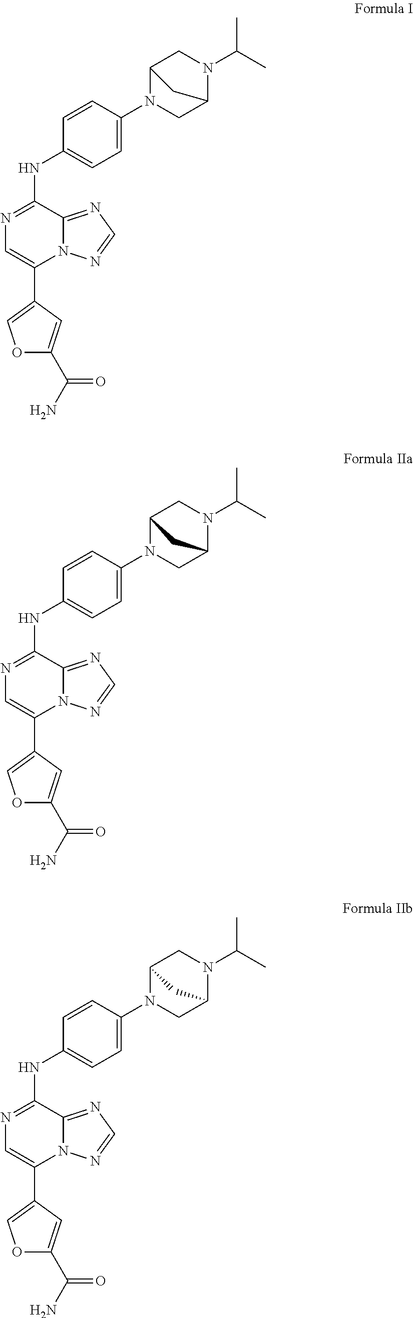
wherein the said salt is a salt formed with adipic acid, L-aspartic acid, benzenesulfonic acid, benzoic acid, caprylic acid, citric acid, fumaric acid, gentisic acid, L-glutamic acid, glycolic acid, hydrochloric acid, L-lactic acid, L-malic acid, maleic acid, L-mandelic acid, methanesulfonic acid, naphthalene-1,5-disulfonic acid, 1-hydroxy-2-naphthoic acid, phosphoric acid, saccharin, succinic acid, sulfuric acid, L-tartaric acid, or toluenesulfonic acid.


SEQUENCE LISTING (6 sequences)

SEQ ID NO: 1 - DNA, 19 bp, artificial
  Target sequence
gctgaccctgaagttcatc

SEQ ID NO: 2 - DNA, 19 bp, Artificial
  Target Sequence
ggttacctaagggtgtggc

SEQ ID NO: 3 - DNA, 19 bp, Artificial
  Target sequence
ctctgagtgcagtgaaatc

SEQ ID NO: 4 - DNA, 19 bp, Artificial
  Target sequence
acaagagcaagatgtggac

SEQ ID NO: 5 - DNA, 19 bp, Artificial
  Target sequence
cggcactttacagagaagc

SEQ ID NO: 6 - DNA, 19 bp, Artificial
  Target sequence
atgatgtgtgccacacacc